(19) United States
(12) Patent Application Publication
(10) Pub. No.: US 2024/0201182 A1
(43) Pub. Date: Jun. 20, 2024

(54) INHIBITION OF PHOTON PHENOMENA ON SINGLE MOLECULE ARRAYS

(71) Applicant: Nautilus Subsidiary, Inc., Seattle, WA (US)

(72) Inventors: Seok Ki CHOI, Palo Alto, CA (US); Michael Augusto DARCY, Fremont, CA (US); Steven TAN, San Mateo, CA (US); Pengyu HAO, Belmont, CA (US)

(21) Appl. No.: 18/540,682

(22) Filed: Dec. 14, 2023

Related U.S. Application Data
(60) Provisional application No. 63/387,555, filed on Dec. 15, 2022.

(51) Int. Cl. G01N 33/543; G01N 33/53

(52) U.S. Cl. CPC G01N 33/54386; G01N 33/5308

(57) ABSTRACT

Methods of providing photodamage inhibitors to single-analyte arrays are described. Photodamage inhibitors may be incorporated into various reagents that are subsequently coupled to single-analyte arrays. Photodamage inhibitors may be replenished during a single-analyte assay through provision of more reagents containing photodamage inhibitors. Useful reagent compositions containing photodamage inhibitors are described.

### CROSS-REFERENCE TO RELATED APPLICATIONS

This application claims priority to U.S. Provisional Application No. 63/387,555, filed on Dec. 15, 2022, which is incorporated herein by reference in its entirety.

### BACKGROUND

Photon-driven phenomena, such as fluorescence and luminescence, provide useful pathways for studying the properties, structures, and interactions of matter. Accordingly, tools such as fluorescence microscopy and luminescence lifetime detection have been developed to leverage photon-driven phenomena for detection of physical system down to the single-molecule or atomic level. In any system that utilizes a photon-driven phenomenon, there exists the possibility of other concurrent photon-related phenomena, some of which may be deleterious to the function and/or performance of the system. Deleterious phenomena, such as singlet oxygen and other reactive oxygen species (ROS) formation, free radical formation, photocatalysis, photolysis, and photocross-linking, can occur in the presence of relatively low-energy photons, such as light within the visible spectrum. Visible light is frequently used for detection in fluorescent and luminescent systems.

Light-based or optical detection is useful for interrogation of array-based systems. Arrays provide an advantageous platform for discretely separating and spacing analytes for individual or clustered analysis. Depending upon a method of optical interrogation used during array-based analysis, individual array sites may be repeatedly exposed to one or more light sources, thereby providing each individual array site a cumulative radiative energy input and a cumulative light exposure time. As both cumulative radiative energy input and cumulative light exposure time increase, the likelihood of a deleterious photon-related reaction occurring for an analyte at an array site may increase.

### SUMMARY

In an aspect, provided herein is a method, comprising: a) providing an array comprising a plurality of sites, in which each site of the plurality of sites comprises one and only one sample polypeptide of a plurality of sample polypeptides, in which the array further comprises a plurality of photodamage inhibitors, and in which photoactive agents of the plurality of photodamage inhibitors are coupled to the array, b) detecting each sample polypeptide of the plurality of sample polypeptides on the array at single-analyte resolution in the presence of an electromagnetic radiation field, in which detecting each sample polypeptide on the array further comprises contacting each site of the plurality of sites with a minimum radiative input from the electromagnetic radiation field of at least 1×10⁻⁶ Joules (J), and c) after contacting the array with the minimum radiative input, detecting at least 90% of sample polypeptides of the plurality of sample polypeptides on the array at single-analyte resolution.

In another aspect, provided herein is a method comprising performing at least 25 cycles of an assay, in which each cycle of the assay comprises the steps of: a) coupling a plurality of photodamage inhibitors to an array, in which the array comprises a plurality of sites, in which each site of the plurality of sites comprises one and only one sample polypeptide of a plurality of sample polypeptides, and in which each site of the plurality of sites is resolvable at single-analyte resolution, b) coupling detectable probes to sample polypeptides of the plurality of sample polypeptides, in which each probe of the detectable probes produces a detectable signal in the presence of an electromagnetic radiation field, and in which each site of the plurality of sites receives a minimum radiative input per cycle of at least 1×10⁻⁶ Joules (J), c) detecting presence or absence of the detectable signal from a probe of the detectable probes at each site of the plurality of sites, in which detecting the presence or absence of the probe comprises providing the electromagnetic radiation field, and d) after providing the electromagnetic radiation field, rinsing at least a fraction of the plurality of photodamage inhibitors from the array.

In another aspect, provided herein is a method, comprising: a) providing an array comprising a plurality of sites, in which each site of the plurality of sites comprises one and only one sample polypeptide of a plurality of sample polypeptides, and in which each site of the plurality of sites is resolvable at single-analyte resolution, b) coupling detectable probes to sample polypeptides of the plurality of sample polypeptides, in which each detectable probe comprises a fluorescent moiety that produces a detectable signal in the presence of an electromagnetic radiation field, in which each detectable probe comprises a nucleic acid nanostructure, and in which each detectable probe further comprises a photodamage inhibitor, c) at each site of the plurality of sites, detecting presence or absence of the detectable signal, in which detecting the presence or absence of the detectable signal comprises providing the electromagnetic radiation field, d) after detecting the presence or absence of the detectable signal, separating the detectable probes from the sample polypeptides, and e) after separating the detectable probes from the sample polypeptides, detecting absence of the detectable signal at each site of the plurality of sites.

In another aspect, provided herein is a method, comprising: a) providing an array comprising a plurality of sites, in which each site of the plurality of sites comprises one and only one sample polypeptide of a plurality of sample polypeptides, in which each site further comprises an anchoring moiety, in which the anchoring moiety comprises a nucleic acid nanostructure, in which the anchoring moiety couples the one and only sample polypeptide to the array, in which the anchoring moiety further comprises a photodamage inhibitor, and in which each site of the plurality of sites is resolvable at single-analyte resolution, b) contacting the array with an electromagnetic radiation field at least two times, and c) after contacting the array with an electromagnetic radiation field at least two times, detecting at each site of the plurality of sites the presence of one and only one polypeptide of the plurality of sample polypeptides.

In another aspect, provided herein is a composition, comprising: a) a nucleic acid nanostructure, in which the nucleic acid nanostructure comprises a first face and a second face, in which the first face and second face comprise differing average orientations, b) a biomolecule covalently coupled to the first face of the nucleic acid nanostructure, and c) a plurality of photodamage inhibitors coupled to the second face of the nucleic acid nanostructure.

In another aspect, provided herein is a composition, comprising: a) a solid support comprising an analyte binding site and an interstitial region, in which the analyte binding site comprises a coupling moiety, b) an analyte coupled to the coupling moiety of the analyte binding site, c) a detectable probe coupled to the analyte, and d) a macromolecular structure coupled to the interstitial region, in which the macromolecular structure comprise a plurality of photodamage inhibitors.

In another aspect, provided herein is an array composition, comprising: a) a solid support comprising a plurality of analyte binding sites, in which each analyte binding site is separated from each other analyte binding site of the plurality of analyte binding sites by one or more interstitial regions, b) a plurality of analytes, in which the plurality of analytes is coupled to the plurality of sites, and in which each site of the plurality of sites comprises one and only one analyte of the plurality of analytes, c) a plurality of detectable probes, in which the plurality of detectable probes is coupled to a subset of the plurality of sites, and d) a plurality of macromolecular structures coupled to the one or more interstitial regions, in which the plurality of probes or the plurality of macromolecular structures comprise photodamage inhibitors.

In an aspect provided herein is a composition, comprising: a) a solid support, b) an anchoring moiety, in which the anchoring moiety is coupled to the solid support, c) an analyte, in which the analyte is coupled to the anchoring moiety, and d) a pendant moiety, in which the pendant moiety comprises a plurality of molecular chains and a plurality of photolabile groups, in which each molecular chain of the plurality of molecular chains is linked to at least one other molecular chain of the plurality of molecular chains by a photolabile group, in which the pendant moiety further comprises a plurality of detectable labels, in which detectable labels are coupled to molecular chains of the plurality of molecular chains, and in which a quantity of detectable labels is proportional to a quantity of photolabile groups.

In another aspect, provided herein is a method, comprising: a) providing a single-analyte array comprising a plurality of sites, in which individual sites of the plurality of sites each comprise one and only one macromolecule of a first plurality of macromolecules, b) binding a second plurality of macromolecules to macromolecules of the first plurality of macromolecules at a fraction of sites of the plurality of sites, thereby forming a macromolecule complex at respective sites of the fraction of sites, in which the macromolecule complex comprises a macromolecule of the second plurality of macromolecules bound to one and only one macromolecule of the first plurality of macromolecules, c) detecting, in the presence of photons, a signal at each individual site of the first fraction of sites, and d) after detecting, in the presence of photons, the signal at respective sites of the first fraction of sites, dissociating macromolecules of the second plurality of macromolecules from macromolecules of the first plurality of macromolecules, in which individual macromolecule complexes each comprise a plurality of photodamage inhibitor moieties that are coupled to the individual macromolecule complex.

In another aspect, provided herein is an array composition, comprising: a) a plurality of sites, in which individual sites of the plurality of sites each comprise one and only one macromolecules of a first plurality of macromolecules, b) at individual sites of a fraction of sites of the plurality of sites, a macromolecule of a second plurality of macromolecules bound to the one and only one macromolecule of the first plurality of macromolecules, and c) at individual sites of the fraction of sites of the plurality of sites, a plurality of photodamage inhibitor moieties coupled to a respective individual site, in which the fraction of sites contains an average of at least 100 photodamage inhibitor moieties per site.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 depicts a single-analyte array composition comprising coupled photodamage inhibitors and uncoupled photodamage inhibitors, in accordance with some embodiments.

FIG. 2 displays a flow chart schematic for a single-analyte process that utilizes photodamage inhibitors to provide photoprotection to single analytes, in accordance with some embodiments.

FIGS. 3A, 3B, 3C, 3D, and 3E illustrate steps of a method of coupling entities comprising photodamage inhibitors to a single-analyte array, in accordance with some embodiments.

FIGS. 4A, 4B, and 4C display steps of a method for coupling entities comprising photodamage inhibitors to an array site, in accordance with some embodiments.

FIGS. 5A, 5B, 5C, and 5D depict cross-sectional views of entities comprising photodamage inhibitors, in accordance with some embodiments.

FIGS. 6A and 6B display configurations of an anchoring moiety comprising photolabile groups before and after exposure to electromagnetic radiation, in accordance with some embodiments.

FIGS. 7A, 7B, and 7C illustrate simulated images of single-analyte signal detection with no photodamage (FIG. 7A), systematic photodamage (FIG. 7B), and stochastic photodamage (FIG. 7C), in accordance with some embodiments.

FIG. 8 shows an array configuration comprising pluralities of different types of photodamage inhibitors with non-uniform spatial distributions, in accordance with some embodiments.

FIG. 9 depicts exemplary photolabile photodamage inhibitors, in accordance with some embodiments.

FIG. 10 displays modes of photon mitigation for photolabile photodamage inhibitors, in accordance with some embodiments.

FIG. 11 illustrates reaction schemes for coupling photolabile photodamage inhibitors to assay agents, in accordance with some embodiments.

FIG. 12 shows reaction schemes for coupling photolabile photodamage inhibitors to DNA intercalating compounds, in accordance with some embodiments.

FIG. 13 depicts exemplary photoisomerization photodamage inhibitors, in accordance with some embodiments.

FIG. 14 displays modes of photon mitigation for photoisomerization photodamage inhibitors, in accordance with some embodiments.

FIG. 15 illustrates reaction schemes for coupling photoisomerization photodamage inhibitors to assay agents, in accordance with some embodiments.

FIG. 16 shows reaction schemes for coupling photoisomerization photodamage inhibitors to DNA intercalating compounds, in accordance with some embodiments.

FIG. 17 depicts exemplary chemical or radical reactive scavenger photodamage inhibitors, in accordance with some embodiments.

FIG. 18 displays modes of chemical or radical species mitigation for chemical or radical reactive scavenger photodamage inhibitors, in accordance with some embodiments.

FIG. 19 illustrates reaction schemes for coupling chemical or radical reactive scavenger photodamage inhibitors to assay agents, in accordance with some embodiments.

FIG. 20 shows reaction schemes for coupling chemical or radical reactive scavenger photodamage inhibitors to assay agents by Click-type reactions, in accordance with some embodiments.

FIG. 21 depicts some examples of single-analyte array components that may form deleterious photon-mediated interactions, in accordance with some embodiments.

FIG. 22 displays a macromolecule complex bound to an array site, in which the macromolecule complex comprises a plurality of photodamage inhibitor moieties, in accordance with some embodiments.

### DETAILED DESCRIPTION

The interrogation and analysis of single-analyte systems differs from that of bulk systems due to an increased measurement uncertainty in the single-analyte systems. Consider, for example, detection of biomolecules in a single-molecule format versus detection of biomolecules in a bulk format. In a bulk analysis, possible false-positive or false-negative detection events will offset each other to some extent, and a relative bias toward false-positive or false-negative detection events can be accounted for when analyzing data. Accordingly, some bulk analyses can be accomplished in a single measurement. On the other hand, measurement certainty cannot always be achieved in a single-molecule format in a single measurement. When interrogating any individual single molecule, true positive or negative detections can be difficult to distinguish from false positive or negative detections based upon a single detection event. Accordingly, single-molecule interrogation and analysis typically employs repeated, sequential, and/or iterative data collection to overcome the inherent measurement uncertainty associated with any single measurement.

Certain single-analyte systems incorporate light-based detection devices to interrogate single-analyte phenomena. Such systems can be configured in an array-based format to achieve single-analyte resolution of individual analytes. Arrays may be advantageous in single-analyte format due to the ability to achieve high surface density of analytes while maintaining sufficient control in the location of each analyte. Light-based detection devices can be configured in array-based single-analyte systems to provide light to and collect light from each single-analyte binding site of an array such that each analyte binding site of the array receives a substantially uniform amount of radiative energy input and/or light exposure time.

Because single-analyte interrogation and analysis often employs multiple measurements to achieve measurement certainty, methods of single-analyte interrogation methods often involve serial or repeated sequences of light-based measurements. For example, an array-based single-analyte detection method that utilizes fluorescence may employ multiple exposures of an array with one or more excitation light sources to measure presence or absence of fluorescent emission at each analyte binding site of the array. With increased cumulative radiative energy input and/or light exposure time, the likelihood of unwanted or deleterious photon-based phenomena increases. At any given analyte binding site, possible deleterious effects from photon-based phenomena can include fragmentation of a single analyte, complete loss of the single analyte from the analyte binding site, unwanted cross-linking of other molecules to the single analyte, or chemical reaction of the single analyte (e.g., photoisomerization, modification due to reaction with singlet oxygen, etc.). Any such alteration of a single analyte can give rise to false negative or false positive detection events during optical detection, thereby impeding single-analyte analysis.

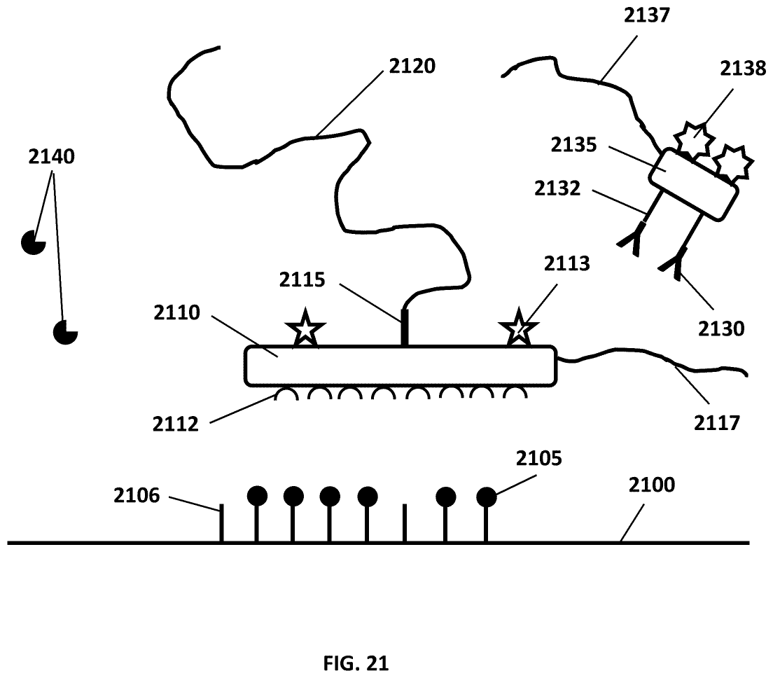

Single-analyte array systems can involve a plurality of differing chemical species that interact during an array-based assay or process. Given the chemical diversity that may be intermingled during a photon-mediated process, numerous pathways may exist for deleterious interactions between the differing chemical species. Moreover, for array system containing millions or potentially billions of unique array sites, even low-probability photon-mediated interactions may be observed when a single-analyte array is detected at single-analyte resolution. FIG. 21 depicts a view of an exemplary site of a single-analyte array system formed on a solid support 2100, in which the macromolecules, small molecules, and surface-coupled molecules may interact, in the presence or absence of photons of light. Each component of the depicted single-analyte system may comprise one or more moieties that can become chemically activated (i.e., reactive) in the presence of photons of light. The solid support 2100 may comprise a material that is reactive with certain species or may be activated by photons of light. The solid support 2100 may further comprise a site containing a layer or coating comprising surface-coupled moieties 2105 that comprise a coupling moiety (i.e., are configured to bind an analyte 2120 or an anchoring moiety 2110). The layer or coating of the site may further comprise one or more surface-coupled moieties 2106 that do not comprise a coupling moiety. The surface-coupled moieties 2106 may contain passivating moieties (e.g., polyethylene glycol moieties, dextran moieties, alkyl moieties, or other molecules that are configured to inhibit unwanted or unexpected binding to the array site), or may comprise defects (e.g., unreacted molecules from a single-step or stepwise synthesis of the layer or coating). The surface coupled moieties 2105 and/or 2106 may comprise one or more moieties that can become chemically activated (i.e., reactive) in the presence of photons of light.

Continuing with FIG. 21, the solid support 2100 or a site thereof may interact with macromolecules (e.g., analytes, anchoring moieties, nanoparticles, binding reagents, etc.). A macromolecule may comprise a chemical species with a molecular weight of 1 kiloDalton (kDa) or higher. As shown in FIG. 21, a macromolecule may comprise a complex containing an analyte 2120 that is attached (e.g., covalently, non-covalently) to an anchoring moiety 2110. The anchoring moiety may comprise various components, including coupling moieties 2112 that are configured to couple to surface-coupled moieties 2105, and a linking moiety 2115 that is configured to attach the analyte 2120 to the anchoring moiety. Optionally, one or more detectable labels 2113 (e.g., fluorophores, luminophores, radiolabels, etc.) may be attached to the analyte 2120 or the anchoring moiety 2110. An anchoring moiety can further comprise an avidity component 2117 that is configured to couple with a complementary avidity component 2137 of a binding reagent. A macromolecule could also comprise a binding reagent. For example, a binding reagent may be configured to bind to an analyte at an array site. An individual binding reagent may comprise an affinity reagent 2130, and optionally a plurality of affinity reagents 2130. A binding reagent may further comprise one or more detectable labels 2138 (e.g., fluorophores, luminophores, radiolabels, etc.). Optionally, a binding reagent can comprise a retaining moiety 2135 (e.g., a nanoparticle, a nucleic acid nanoparticle) that couples binding reagent components (affinity reagents 2130, detectable labels 2138, etc.). Optionally, an affinity reagent 2130 may be attached (e.g., covalently, non-covalently) to a retaining moiety 2135 by a linking moiety 2132. Additional examples of macromolecules can include enzymes, non-nucleic acid polypeptides, nanoparticles, polymeric molecules, etc. It will be recognized that the numerous macromolecules species or components thereof described herein may comprise one or more moieties that can become chemically activated (i.e., reactive) in the presence of photons of light.

Continuing with FIG. 21, the solid support 2100, a site thereof, or a component attached thereto may interact with a small molecule 2140. A small molecule 2140 may comprise a chemical species with a molecular weight of less than about 1 kiloDalton (kDa). Small molecules can include chemical reagents (e.g., chemical reactants, chemically inert species), pharmaceutical compounds, metabolites, or excipient agents (e.g., buffering species, surfactants, detergents, denaturants, chaotropes, crowding agents, biocidal agents, thermoprotectants, etc.). It will be recognized that the numerous small molecule species or moieties thereof (e.g., functional groups) described herein may comprise one or more moieties that can become chemically activated (i.e., reactive) in the presence of photons of light.

Table I presents possible unwanted or unexpected binding interactions that may be mediated by photons (e.g., photon-mediated cross-linking, photon-mediated fragmentation followed by reaction of a fragment with another system component, photon-mediated generation of free radical species followed by reaction of the radical species with another system component, etc.) with exemplary and non-limiting reference to the reference numerals of FIG. 21. A moiety from a first system component listed in column 1 may form an interaction with a second system component listed in column 2 due to a photon-mediated chemical binding interaction, or due to a binding interaction after a moiety of the first system component or the second system component is activated by a photon. In some cases, a molecule may become coupled to itself due to a photon-mediated interaction. It should be noted that certain first components and second components listed in Table I may be designed or expected to form wanted or expected interactions. For example, an anchoring moiety 2110 may be configured to be bound to an array site by a binding interaction between a coupling moiety 2112 and a surface-coupled moiety 2105. However, a photon-mediated interaction may form an interaction between the anchoring moiety 2110 and the surface-coupled moiety 2105, thereby orienting the anchoring moiety 2110 in an unintended configuration (e.g., partially or entirely occluding the analyte 2120, orienting the analyte 2120 toward the surface-coupled moieties 2105 or solid support 2100, etc.). Likewise, an affinity agent 2130 of a binding reagent may be configured to bind to an analyte 2120, but a photon-mediated interaction may cross-link the affinity agent 2130 to the analyte 2120, thereby inhibiting dissociation of the affinity agent 2130 from the analyte 2120.

TABLE I
Possible Partners for Unwanted or Deleterious
Photon-Mediated Interactions

| First Component | Possible Second Components |
|---|---|
| 2100 | 2105, 2106, 2110, 2112, 2113, 2115, 2117, |
| | 2120, 2130, 2132, 2135, 2137, 2138, 2140 |
| 2105 | 2100, 2105, 2106, 2110, 2112, 2113, 2115, 2117, |
| | 2120, 2130, 2132, 2135, 2137, 2138, 2140 |
| 2106 | 2100, 2105, 2106, 2110, 2112, 2113, 2115, 2117, |
| | 2120, 2130, 2132, 2135, 2137, 2138, 2140 |
| 2110 | 2100, 2105, 2106, 2110, 2112, 2113, 2115, 2117, |
| | 2120, 2130, 2132, 2135, 2137, 2138, 2140 |
| 2112 | 2100, 2105, 2106, 2110, 2112, 2113, 2115, 2117, |
| | 2120, 2130, 2132, 2135, 2137, 2138, 2140 |
| 2113 | 2100, 2105, 2106, 2110, 2112, 2113, 2115, 2117, |
| | 2120, 2130, 2132, 2135, 2137, 2138, 2140 |
| 2115 | 2100, 2105, 2106, 2110, 2112, 2113, 2115, 2117, |
| | 2120, 2130, 2132, 2135, 2137, 2138, 2140 |
| 2117 | 2100, 2105, 2106, 2110, 2112, 2113, 2115, 2117, |
| | 2120, 2130, 2132, 2135, 2137, 2138, 2140 |
| 2120 | 2100, 2105, 2106, 2110, 2112, 2113, 2115, 2117, |
| | 2120, 2130, 2132, 2135, 2137, 2138, 2140 |
| 2130 | 2100, 2105, 2106, 2110, 2112, 2113, 2115, 2117, |
| | 2120, 2130, 2132, 2135, 2137, 2138, 2140 |
| 2132 | 2100, 2105, 2106, 2110, 2112, 2113, 2115, 2117, |
| | 2120, 2130, 2132, 2135, 2137, 2138, 2140 |
| 2135 | 2100, 2105, 2106, 2110, 2112, 2113, 2115, 2117, |
| | 2120, 2130, 2132, 2135, 2137, 2138, 2140 |
| 2137 | 2100, 2105, 2106, 2110, 2112, 2113, 2115, 2117, |
| | 2120, 2130, 2132, 2135, 2137, 2138, 2140 |
| 2138 | 2100, 2105, 2106, 2110, 2112, 2113, 2115, 2117, |
| | 2120, 2130, 2132, 2135, 2137, 2138, 2140 |
| 2140 | 2100, 2105, 2106, 2110, 2112, 2113, 2115, 2117, |
| | 2120, 2130, 2132, 2135, 2137, 2138, 2140 |

Provided herein are methods and systems for inhibiting deleterious interactions of photons with single analytes in array-based single-analyte systems. The methods set forth herein may involve the introduction of molecules that include photodamage inhibitors before or during single-analyte assays. In some embodiments, the molecules including photodamage inhibitors may be coupled to an array in sufficient proximity to single analytes to inhibit the likelihood of photodamage for any individual single analyte. In certain embodiments, the quantity of available photodamage inhibitors coupled to an array may be replenished during a single-analyte assay. For example, photodamage inhibitors can be delivered multiple times during the course of a multicycle assay, such that the newly delivered photodamage inhibitors couple to the array to replenish the quantity of available photodamage inhibitors coupled to the array.

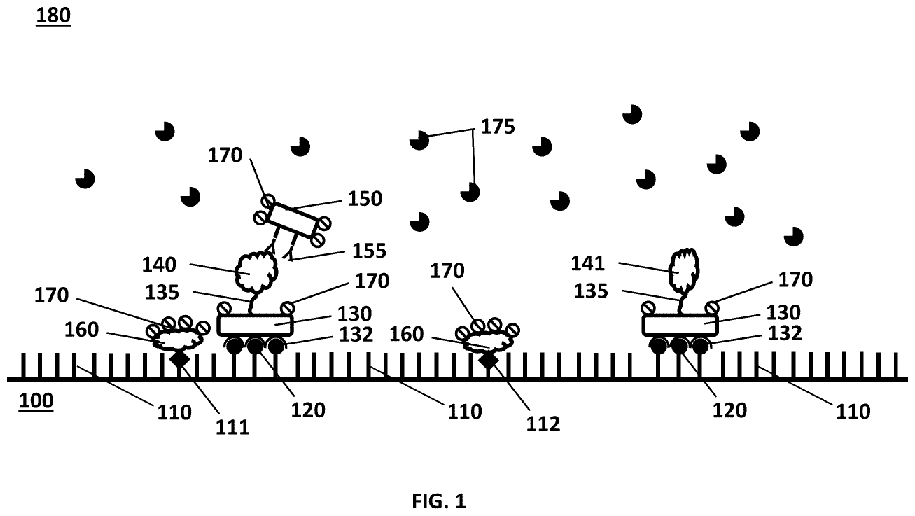

FIG. 1 depicts a single-analyte array composition, in accordance with some embodiments. The single-analyte array composition comprises photodamage inhibitors that are coupled to the array using various array components. The array comprises a solid support 100 that comprises analyte binding sites separated by interstitial regions. The analyte binding sites comprises coupling moieties 120 that anchor analytes to the solid support 100. The interstitial regions comprise passivating moieties 110 that are configured to inhibit binding of moieties to the interstitial regions. The interstitial regions also comprise defects 111 and 112 that are passivated by coupled macromolecular structures 160. Single analytes 140 and 141 are coupled to different analyte binding sites by anchoring moieties 130 (e.g., nucleic acid nanoparticles). The anchoring moieties comprise complementary coupling moieties 132 that couple to coupling moieties 120. The anchoring moieties 130 further comprise an optional linker 135 that attaches the analytes 140 and 141 to respective anchoring moieties 130. A detectable probe is coupled to analyte 140. The detectable probe comprises a plurality of affinity agents 155 that is attached to a retaining component 150 (e.g., a nucleic acid nanoparticle). Each of the anchoring moieties 130, macromolecular structures 160, and retaining component 150 comprise incorporated photodamage inhibitors 170. The single-analyte array is optionally contacted with a fluidic medium 180 that comprises a plurality of unincorporated photodamage inhibitors 175 (e.g., solvated or suspended in solution within the fluidic medium 180). Given the closer proximity of defect 111 to the analyte binding site comprising analyte 140 relative to the proximity of defect 112 to the analyte binding site comprising analyte 141, the incorporated photodamage inhibitors 170 that are co-located with the macromolecular structure 160 at defect 111 may be more likely to provide a degree of photodamage protection to analyte 140 than the incorporated photodamage inhibitors 170 that are co-located with the macromolecular structure 160 at defect 112 for analyte 141.

### Definitions

As used herein, the terms “address,” “site,” or “array site” refer synonymously to a location in an array where a particular molecule (e.g. organic molecule, inorganic molecule, passivating moiety, analyte, etc.) is present or is configured to be bound. An address can contain a single molecule, or it can contain a population of several molecules of the same species (i.e. an ensemble of the analytes). Alternatively, an address can include a population of different molecules. Addresses are typically discrete. The discrete addresses can be contiguous, or they can be separated by interstitial spaces. An address may be vacant (e.g., an array site that is configured to bind a molecule but has not bound the molecule). An array useful herein can have, for example, addresses that are separated by less than 100 microns, 10 microns, 1 micron, 100 nm, 10 nm or less. Alternatively or additionally, an array can have addresses that are separated by at least 10 nm, 100 nm, 1 micron, 10 microns, or 100 microns. The addresses can each have an area of less than 1 square millimeter, 500 square microns, 100 square microns, 10 square microns, 1 square micron, 100 square nm or less. An array can include at least about 1×10⁴, 1×10⁵, 1×10⁶, 1×10⁷, 1×10⁸, 1×10⁹, 1×10¹⁰, 1×10¹¹, 1×10¹², or more addresses. A site can have a characteristic dimension (e.g., a diameter, a length, a width, a circumference) of at least about 1 nm, 10 nm, 100 nm, 200 nm, 300 nm, 400 nm, 500 nm, 1 micron, 1.5 microns, 2 microns, 5 microns, or more than 5 microns. Alternatively or additionally, a site can have a characteristic dimension of no more than about 5 microns, 2 microns, 1.5 microns, 1 micron, 500 nm, 400 nm, 300 nm, 200 nm, 100 nm, 10 nm, 1 nm, or less than 1 nm.

As used herein, the term “affinity agent” refers to a molecule or other substance that is capable of specifically or reproducibly binding to an analyte (e.g. protein). An affinity agent can be larger than, smaller than or the same size as the analyte. An affinity agent may form a reversible or irreversible bond with an analyte. An affinity agent may bind with an analyte in a covalent or non-covalent manner. Affinity agents may include reactive affinity agents, catalytic affinity agents (e.g., kinases, proteases, etc.) or non-reactive affinity agents (e.g., antibodies or fragments thereof). An affinity agent can be non-reactive and non-catalytic, thereby not permanently altering the chemical structure of an analyte to which it binds. Affinity agents that can be particularly useful for binding to proteins include, but are not limited to, antibodies or functional fragments thereof (e.g., Fab′ fragments, F(ab′)2 fragments, single-chain variable fragments (scFv), di-scFv, tri-scFv, or microantibodies), affibodies, affilins, affimers, affitins, alphabodies, anticalins, avimers, DARPins, monobodies, nanobodies, nanoCLAMPs, nucleic acid aptamers, protein aptamers, lectins or functional fragments thereof.

As used herein, the term “anchoring moiety” refers to a molecule, particle, or other moiety that serves as an intermediary attaching a protein, peptide or other analyte of interest to a surface (e.g., a solid support or a microbead). An anchoring moiety may be covalently or non-covalently attached to a surface and/or analyte of interest. An anchoring moiety may be a biomolecule, polymer, particle, nanoparticle, or any other entity that is capable of attaching to a surface or analyte of interest. In some cases, an anchoring moiety may be a nucleic acid nanoparticle, such as a structured nucleic acid particle. In some cases, an anchoring moiety may occlude contact between an analyte attached to the anchoring moiety and a surface (e.g., a solid support or array surface).

As used herein, the term “analyte” refers to a molecule, particle, or complex of molecules or particles that is coupled or is to be coupled to an array or a site thereof. An analyte may comprise a target for an analytical method (e.g., sequencing, identification, quantification, etc.) or may comprise a functional element such as a binding ligand or a catalyst. An analyte may comprise a biomolecule, such as a polypeptide, polysaccharide, nucleic acid, lipid, metabolite, enzyme cofactor, pharmaceutical agent, or a combination thereof. An analyte may comprise a non-biological molecule, such as a polymer, metal, metal oxide, ceramic, semiconductor, mineral, or a combination thereof.

As used herein, the term “array” refers to a population of sites that are associated with unique identifiers such that the sites can be distinguished from each other. An array may include an array of analytes, in which each array site contains at least one analyte. A unique identifier can be, for example, a solid support (e.g., particle or bead), address on a solid support, position relative to a fiducial maker, tag, label (e.g., luminophore), or barcode (e.g., nucleic acid barcode) that is associated with a site and that is distinct from other identifiers in the array. Sites can be associated with unique identifiers by attachment, for example, via covalent bonds or non-covalent bonds (e.g., ionic bond, hydrogen bond, van der Waals forces, electrostatics etc.). Unique identifiers can be attached to analytes that are present at array sites. An array can include different analytes that are each attached to different unique identifiers. An array can include different unique identifiers that are attached to the same or similar analytes. An array can include separate solid supports or separate addresses that each bear a different analyte, in which the different analytes can be identified according to the locations of the solid supports or addresses.

As used herein, the terms “attached,” “bound,” or “coupled” refer to the state of two things being joined, coupled, fastened, adhered, or connected to each other. Attachment, coupling, or binding can be covalent or non-covalent. For example, a particle can be attached to a protein by a covalent or non-covalent bond. A covalent bond is characterized by the sharing of pairs of electrons between atoms. A non-covalent bond is a chemical bond that does not involve the sharing of pairs of electrons and can include, for example, hydrogen bonds, ionic bonds, van der Waals forces, pi-pi stacking, hydrophilic interactions, adhesion, adsorption, and hydrophobic interactions.

As used herein, the term “click-type reaction” refers to a single-step, thermodynamically-favorable conjugation reaction utilizing biocompatible reagents. A click reaction may be configured to not utilize toxic or biologically incompatible reagents (e.g., acids, bases, heavy metals) or to not generate toxic or biologically incompatible byproducts. A click reaction may utilize an aqueous solvent or buffer (e.g., phosphate buffer solution, Tris buffer, saline buffer, MOPS, etc.). A click reaction may be thermodynamically favorable if it has a negative Gibbs free energy of reaction, for example a Gibbs free energy of reaction of less than about—5 kiloJoules/mole (kJ/mol), −10 kJ/mol, −25 kJ/mol, −100 kJ/mol,−250 kJ/mol, −500 kJ/mol, or less. Exemplary click reactions may include metal-catalyzed azide-alkyne Huisgen cycloaddition, strain-promoted azide-alkyne cycloaddition, strain-promoted azide-nitrone cycloaddition, strained alkene reactions, thiol-ene reaction, Diels-Alder reaction, inverse electron demand Diels-Alder reaction (IEDDA), [3+2] cycloaddition, [4+1] cycloaddition, nucleophilic substitution, dihydroxylation, thiol-yne reaction, photo thiol-yne reaction, photo thiol-ene reaction, photoclick, nitrone dipole cycloaddition, norbornene cycloaddition, oxanobornadiene cycloaddition, tetrazine ligation, and tetrazole photoclick reactions. Exemplary reactive moieties utilized to perform click reactions may include alkenes, alkynes, azides, epoxides, amines, thiols, nitrones, isonitriles, isocyanides, aziridines, activated esters, and tetrazines. Other well-known click conjugation reactions may be used having complementary bioorthogonal reaction species, for example, where a first click component comprises a hydrazine moiety and a second click component comprises an aldehyde or ketone group, and where the product of such a reaction comprises a hydrazone functional group or equivalent. Exemplary bioorthogonal and click reactions are set forth in U.S. Pat. App. Pub. No. 2021/0101930 A1, which is incorporated herein by reference.

The term “comprising” is intended herein to be open-ended, including not only the recited elements, but further encompassing any additional elements.

As used herein, the term “defect,” when used in reference to an interstitial region or an analyte binding site of an array, refers to an address containing a chemical irregularity with respect to a bulk characteristic or structure of the interstitial region or an analyte binding site. A chemical irregularity with respect to an interstitial region or an analyte binding site may include absence of a passivating molecule, or plurality of passivating molecules, at an address of a surface that is configured to contain a passivating layer. A chemical irregularity with respect to an interstitial region or an analyte binding site may include an increased or decreased concentration of molecules at an address of a surface relative to an average concentration of molecules for a passivating layer. A defect may comprise a void in a passivating layer at an interstitial region or an analyte binding site. For example, a passivating layer on a surface of a solid support may comprise a void (e.g., an absence of a molecule, particle, or moiety) that permits direct contact between an assay agent and the surface of the solid support. A defect may comprise a molecule, particle, or moiety whose chemical structure or characteristics differ from the bulk chemical structure or characteristics of an interstitial region or an analyte binding site. For example, a passivating layer of polyethylene glycol (PEG) molecules on a surface of an interstitial region may comprise a defect containing a non-PEGylated molecule. A defect in a passivating layer may contain a molecule, particle, or moiety that facilitates binding of an assay agent to the passivating layer, such as a reactive species, an electrically-charged species, a magnetic species, a polar species, or a combination thereof. A defect may comprise a molecule, particle, or moiety that is covalently bound to a surface containing a passivating layer. A defect may comprise a molecule, particle, or moiety that is non-covalently bound to a surface containing a passivating layer.

As used herein, the term “each,” when used in reference to a collection of items, is intended to identify an individual item in the collection but does not necessarily refer to every item in the collection. Exceptions can occur if explicit disclosure or context clearly dictates otherwise.

As used herein, the term “fluidic medium” refers to a fluid that is configured to be contacted with a single-analyte array, as set forth herein. A fluidic medium may comprise a liquid fluid medium or a gas fluidic medium. A fluidic medium may comprise any of a variety of components, such as a solvent species, pH buffering species, a cationic species, an anionic species, a surfactant species, a denaturing species, or a combination thereof. A solvent species may include water, acetic acid, methanol, ethanol, n-propanol, isopropyl alcohol, n-butanol, formic acid, ammonia, propylene carbonate, nitromethane, dimethyl sulfoxide, acetonitrile, dimethylformamide, acetone, ethyl acetate, tetrahydrofuran, dichloromethane, chloroform, carbon tetrachloride, dimethyl ether, diethyl ether, 1,4-dioxane, toluene, benzene, cyclohexane, hexane, cyclopentane, pentane, or combinations thereof. A fluidic medium may include a buffering species including, but not limited to, MES, Tris, Bis-tris, Bis-tris propane, ADA, ACES, PIPES, MOPSO, MOPS, BES, TES, HEPES, HEPBS, HEPPSO, DIPSO, MOBS, TAPSO, TAPS, TABS, POPSO, TEA, EPPS, Tricine, Gly-Gly, Bicine, AMPD, AMPSO, AMP, CHES, CAPSO, CAPS, PBS, and CABS. A fluidic medium may comprise a cationic species such as Na⁺, K⁺, Ag⁺, Cu⁺, NH₄⁺, Mg²⁺, Ca²⁺, Cu²⁺, Cd²⁺, Zn²⁺, Fe²⁺, Co²⁺, Niz⁺, Cr²⁺, Mn²⁺, Ge²⁺, Sn²⁺, A13⁺, Cr³⁺, Fe3⁺, Co³⁺, Ni³⁺, Ti³⁺, Mn³⁺, Si⁴⁺, V⁴⁺, Ti⁴⁺, Mn⁴⁺, Ge⁴⁺, Se4⁺, V⁵⁺, Mn⁵⁺, Mn⁶⁺, Se⁶⁺, and combinations thereof. A fluidic medium may comprise an anionic species such as F⁻, Cl⁻, Br⁻, ClO₃⁻, H₂PO₄⁻, HCO₃⁻, HSO₄⁻, OH⁻, I⁻, NO₃⁻, NO₂⁻, MnO₄⁻, SCN⁻, CO₃²⁻, CrO₄²⁻, Cr₂O₇²⁻, HPO₄²⁻, SO₄²⁻, SO₃²⁻, PO₄³⁻, and combinations thereof. A fluidic medium may include a surfactant species, such as a cationic surfactant, an anionic surfactant, a non-ionic surfactant, a zwitterionic surfactant, or an amphoteric surfactant. A fluidic medium may include a surfactant species including, but not limited to, stearic acid, lauric acid, oleic acid, sodium dodecyl sulfate, sodium dodecyl benzene sulfonate, dodecylamine hydrochloride, hexadecyltrimethylammonium bromide, polyethylene oxide, nonylphenyl ethoxylates, Triton X, pentapropylene glycol monododecyl ether, octapropylene glycol monododecyl ether, pentaethylene glycol monododecyl ether, octaethylene glycol monododecyl ether, lauramide monoethylamine, lauramide diethylamine, octyl glucoside, decyl glucoside, lauryl glucoside, Tween 20, Tween 80, n-dodecyl-β-D-maltoside, nonoxynol 9, glycerol monolaurate, polyethoxylated tallow amine, poloxamer, digitonin, zonyl FSO, 2,5-dimethyl-3-hexyne-2,5-diol, Igepal CA630, Aerosol-OT, triethylamine hydrochloride, cetrimonium bromide, benzethonium chloride, octenidine dihydrochloride, cetylpyridinium chloride, adogen, dimethyldioctadecylammonium chloride, CHAPS, CHAPSO, cocamidopropyl betaine, amidosulfobetaine-16, lauryl-N,N-(dimethylammonio)butyrate, lauryl-N,N-(dimethyl)-glycinebetaine, hexadecyl phosphocholine, lauryldimethylamine N-oxide, lauryl-N,N-(dimethyl)-propanesulfonate, 3-(1-pyridinio)-1-propanesulfonate, 3-(4-tert-butyl-1-pyridinio)-1-propanesulfonate, N-laurylsarcosine, and combinations thereof.

As used herein, the terms “group” and “moiety” are intended to be synonymous when used in reference to the structure of a molecule. The terms refer to a component or part of the molecule. The terms do not necessarily denote the relative size of the component or part compared to the rest of the molecule, unless indicated otherwise.

As used herein, the term “label” and refers to a molecule or moiety that provides a detectable characteristic. The detectable characteristic can be, for example, an optical signal such as absorbance of radiation, luminescence emission, luminescence lifetime, luminescence polarization, fluorescence emission, fluorescence lifetime, fluorescence polarization, or the like; Rayleigh and/or Mie scattering; binding affinity for a ligand or receptor; magnetic properties; electrical properties; charge; mass; radioactivity or the like. Exemplary labels include, without limitation, a fluorophore, luminophore, chromophore, nanoparticle (e.g., gold, silver, carbon nanotubes), heavy atoms, radioactive isotope, mass label, charge label, spin label, receptor, ligand, or the like. A label may produce a signal that is detectable in real-time (e.g., fluorescence, luminescence, radioactivity). A label may produce a signal that is detected off-line (e.g., a nucleic acid barcode) or in a time-resolved manner (e.g., time-resolved fluorescence). A label may produce a signal with a characteristic frequency, intensity, polarity, duration, wavelength, sequence, or fingerprint.

As used herein, the terms “linker” and “linking moiety” refer synonymously to a moiety that connects two objects to each other. One or both objects can be a molecule, solid support, address, particle or bead. Both objects can be moieties of a molecule, solid support, address, particle or bead. The term can also refer to an atom, moiety or molecule that is configured to react with two objects to form a moiety that connects the two objects. The connection of a linker to one or both objects can be a covalent bond or non-covalent bond. A linker may be configured to provide a chemical or mechanical property to the moiety connecting two objects, such as hydrophobicity, hydrophilicity, electrical charge, polarity, rigidity, or flexibility. A linker may comprise two or more functional groups that facilitate coupling of the linker to the first and second objects. A linker may include a polyfunctional linker such as a homobifunctional linker, heterobifunctional linker, homopolyfunctional linker, or heteropolyfunctional linker. A linker may comprise a molecular or polymeric chain. Exemplary compositions for linkers can include, but are not limited to, a polyethylene glycol (PEG), polyethylene oxide (PEO), amino acid, protein, nucleotide, nucleic acid, nucleic acid origami, dendrimer, protein nucleic acid (PNA), polysaccharide, carbon, nitrogen, oxygen, ether, sulfur, or disulfide. A linker can be a bead or particle such as a structured nucleic acid particle.

As used herein, the term “macromolecule” refers to a molecule or complex of molecules having a molecular weight greater than or equal to 1 kiloDalton (kDa). A macromolecule can include complexes of small molecules, such as hybridized groups of oligonucleotides, provided the complex has a molecular weight of at least 1 kDa. A macromolecule can include two or more macromolecules that are attached (e.g., covalently, non-covalently) into a single structure. For example, a macromolecule may comprise an analyte coupled to an anchoring moiety, as set forth herein. As used herein, the term “small molecule” refers to a molecule or complex of molecules having a molecular weight of less than 1 kDa.

As used herein, the term “nanoparticle” refers to a discrete entity having a characteristic dimension (e.g., diameter, length, width) of less than 1 micron. A nanoparticle can include a complex of smaller molecules, particles, or moieties that maintain an association with each other. For example, a nanoparticle can comprise a hybridized complex or cluster of nucleic acids. A nanoparticle can comprise an inorganic material, such as a metal, metal oxide, metal nitride, metal carbide, metal sulfide, or semiconductor material. An inorganic nanoparticle may further comprise organic functional groups that facilitate attachment of moieties to the nanoparticle. A nanoparticle can comprise an organic material, such as a carbon nanoparticle (e.g., carbon nanosphere, carbon nanotubes, carbon onions, etc.) or a polymeric nanoparticle. A polymeric nanoparticle can include linear polymers, branched polymers, or dendrimeric polymers. A polymeric nanoparticle may comprise a biopolymer such as a nucleic acid nanoparticle, a polypeptide nanoparticle, or a polysaccharide nanoparticle.

As used herein, the terms “nucleic acid nanostructure” and “nucleic acid nanoparticle,” refer synonymously to a single- or multi-chain polynucleotide molecule comprising a compacted three-dimensional structure. The compacted three-dimensional structure can optionally have a characteristic tertiary structure. An exemplary nucleic acid nanostructure is a structured nucleic acid particle (SNAP). A SNAP can be configured to have an increased number of interactions between regions of a polynucleotide strand, less distance between the regions, increased number of bends in the strand, and/or more acute bends in the strand, as compared to the same nucleic acid molecule in a random coil or other non-structured state. Alternatively or additionally, the compacted three-dimensional structure of a nucleic acid nanostructure can optionally have a characteristic quaternary structure. For example, a nucleic acid nanostructure can be configured to have an increased number of interactions between polynucleotide strands or less distance between the strands, as compared to the same nucleic acid molecule in a random coil or other non-structured state. In some configurations, the tertiary structure (i.e. the helical twist or direction of the polynucleotide strand) of a nucleic acid nanostructure can be configured to be more dense than the same nucleic acid molecule in a random coil or other non-structured state. Nucleic acid nanostructures may include deoxyribonucleic acid (DNA), ribonucleic acid (RNA), peptide nucleic acid (PNA), other nucleic acid analogs, and combinations thereof. Nucleic acid nanostructures may have naturally-arising or engineered secondary, tertiary, or quaternary structures. A structured nucleic acid particle can contain at least one of: i) a moiety that is configured to couple an analyte to the nucleic acid nanostructure, ii) a moiety that is configured to couple the nucleic acid nanostructure to another object such as another SNAP, a solid support or a surface thereof, iii) a moiety that is configured to provide a chemical or physical property or characteristic to a nucleic acid nanostructure, or iv) a combination thereof. Exemplary SNAPs may include nucleic acid nanoballs (e.g. DNA nanoballs), nucleic acid nanotubes (e.g. DNA nanotubes), and nucleic acid origami (e.g. DNA origami). A SNAP may be functionalized to include one or more reactive handles or other moieties. A SNAP may comprise one or more incorporated residues that contain reactive handles or other moieties (e.g., modified nucleotides).

As used herein, the term “nucleic acid origami” refers to a nucleic acid construct having an engineered tertiary or quaternary structure. A nucleic acid origami may include DNA, RNA, PNA, modified or non-natural nucleic acids, or combinations thereof. A nucleic acid origami may include a plurality of oligonucleotides that hybridize via sequence complementarity to produce the engineered structuring of the origami. A nucleic acid origami may include sections of single-stranded or double-stranded nucleic acid, or combinations thereof. Exemplary nucleic acid origami structures may include nanotubes, nanowires, cages, tiles, nanospheres, blocks, and combinations thereof. A nucleic acid origami can optionally include a relatively long scaffold nucleic acid to which multiple smaller nucleic acids hybridize, thereby creating folds and bends in the scaffold that produce an engineered structure. The scaffold nucleic acid can be circular or linear. The scaffold nucleic acid can be single stranded but for hybridization to the smaller nucleic acids. A smaller nucleic acid (sometimes referred to as a “staple”) can hybridize to two regions of the scaffold, in which the two regions of the scaffold are separated by an intervening region that does not hybridize to the smaller nucleic acid.

As used herein, the term “optically resolvable distance” refers to a distance on an array or a surface thereof at which two separate objects can be optically distinguished with respect to each other. The threshold for an optically resolvable distance can vary based upon a mechanism of detection and/or the physical apparatus used to perform an optical detection as well as a detectable species utilized for detection (e.g., single fluorophores, multiple fluorophores, nanoparticles, intercalated dyes, etc.). For example, when detecting two fluorescent objects on a surface via optical microscopy, an optically resolvable distance may depend upon an excitation wavelength of fluorophores, an emission wavelength of fluorophores, and optical characteristics of an optical microscope utilized to image the objects. An optically resolvable distance may be at least about 1 nanometer (nm), 5 nm, 10 nm, 25 nm, 50 nm, 75 nm, 100 nm, 125 nm, 150 nm, 200 nm, 300 nm, 400 nm, 500 nm, or more than 500 nm. Alternatively or additionally, an optically resolvable distance may be no more than about 500 nm, 400 nm, 300 nm, 200 nm, 150 nm, 125 nm, 100 nm, 75 nm, 50 nm, 25 nm, 10 nm, 5 nm, 1 nm, or less than 1 nm. In some cases, an optically resolvable distance may be determined with respect to a detection method (e.g., a pixel-based sensor). For example, two objects may be considered to be separated by an optically resolvable distance if a sensor-based detection produces two optical signal intensity maxima (corresponding to the two objects) and an optical signal intensity minimum between the two maxima, in which the optical signal intensity minimum has a magnitude that is no more than half of the average signal-to-noise ratio of the two optical signal intensity maxima. As used herein, the term “optically non-resolvable distance” refers to a distance on an array or a surface thereof which is less than an optically resolvable distance, as set forth herein. An optically non-resolvable distance may be a distance at which an optical signal from a first object can not be distinguished from an optical signal from a second object. For example, a first optical signal from a first object may be optically non-resolvable from a second optical signal from a second object if the first optical signal and the second optical signal are respectively detected by adjacent pixels of a pixel-based sensor.

As used herein, the term “orthogonal binding”, when used in reference to an array, refers to an unwanted, unexpected, or contrary-to-design binding interaction that occurs between an array surface or array feature and an unbound moiety that may become contacted with the array surface or array feature. Orthogonal binding may be qualitatively characterized as a binding interaction that occurs in a system that has been engineered to prevent such a binding interaction (e.g., a hydrophilic molecule binding to a putatively hydrophobic surface). Orthogonal binding may be quantitatively characterized as measurable binding interactions occurring between an array surface or array feature (e.g., an interstitial region, an analyte binding site) and an unbound moiety that may become contacted with the array surface or feature, in which the measurable binding interactions occur at a rate and/or to an extent that exceeds a predicted rate and/or extent, such as a thermodynamic or kinetic prediction (e.g., a dissociation constant, a binding on-rate, a binding off-rate, etc.). For example, if an unbound moiety is characterized to bind to a surface-coupled passivating moiety (e.g., polyethylene glycol) with a kilomolar dissociation constant (a very weak binding interaction), then observing a millimolar binding dissociation constant between the unbound moiety and an array surface that is provided with a uniform layer of the surface-coupled passivating moiety would indicate a orthogonal binding phenomena (i.e., binding due to a mechanism other than the specific binding of the unbound moiety to the surface-coupling passivating moiety). Orthogonal binding may be characterized based upon a stochastic measure, such as spatial and/or temporal variations in unwanted, unexpected, or contrary-to-design binding phenomena.

As used herein, the term “passivate,” when used in reference to an array or a surface thereof, refers to inhibition of unwanted binding of an assay agent to the array or the surface thereof. A layer, molecule, particle, or moiety on an array or a surface thereof can be considered passivating with respect to a binding context to which the array or array surface is exposed. For example, a surface comprising a plurality of attached oligonucleotides with uniform nucleotide sequences would be expected to specifically bind oligonucleotide assay agents with complementary sequences to the attached oligonucleotides (i.e., a wanted or intended binding interaction), but would inhibit binding of oligonucleotide assay agents with partially or completely non-complementary nucleotide sequences (i.e., unwanted or unintended binding interactions). Likewise, a surface comprising a layer of positively-charged amine moieties (i.e., a positively charged surface) can be expected to form binding interactions with negatively-charged assay agents, and would be expected to inhibit binding of positively-charged assay agents.

As used herein, the term “pendant” when used in reference to a moiety coupled to an entity, refers to the moiety possessing one or more degrees of freedom of motion with respect to the entity (e.g., a particle or nanoparticle). A pendant moiety may comprise a moiety comprising a molecular chain, in which one terminus of the molecular chain is coupled to an entity and a second terminus is uncoupled to the entity. A pendant moiety may comprise a moiety comprising a molecular chain, in which both termini of the molecular chain are coupled to an entity and an intermediate moiety of the molecular chain is uncoupled to the entity. A length of a pendant moiety comprising a molecular chain may exceed a persistence length of the molecular chain, for example by at least about a factor of 1.1, 1.5, 2, 3, 4, 5, 10, 25, 50, 100, 500, 1000, or more than 1000.

As used herein, the term “photodamage inhibitor” refers to any suitable molecule, covalent conjugate thereof, or non-covalent complexes thereof, that is characterized by an ability to absorb, transfer, emit or otherwise respond to photons, deactivate photo-generated chemical species, or combinations thereof. Photodamge inhibitors may comprise light-responsive molecules or light-absorbing chromophore molecules, chemical scavenging molecules, free radical scavenging molecules, their derivatives, or covalent conjugates thereof with macromolecules. Light-responsive molecules can include photo-cleavable molecules, photoisomerization molecules, and molecules comprising such molecules. In some cases, photo-cleavable molecules may refer to molecules that have an ability to undergo one or more bond fragmentations upon activation by photon absorption. Photo-cleavable molecules can include but are not limited certain chromophores and their derivatives such as coumarin, ortho-nitrobenzyl, ortho-nitrobenzofuran, quinolinone, carbazole or benzoin. Photoisomerization molecules and systems may be characterized by a mechanism of bond isomerization which occurs in a reversible process. Such bond isomerization facilitates reversible photon scavenging, and is distinguished from photon cleavage that occurs in an irreversible manner through self-fragmentation or self-immolation of photocleavable molecules. Examples of photoisomerization include trans to cis-azobenzene, trans to cis-stilbene, merocyanin to spiropyran, diarylethene, isomerization of single or multiple double bonds in retinol and thioindigo, and donor-acceptor Stenhouser adduct (DASA). Chemical or radical scavenging molecules may indirectly respond to a presence of photons by deactivating reactive oxygen species (ROS), free radical ions, and any other conceivable reactive species that can be generated in the detection system by excess photons. Photon scavenging molecules also include those that can scavenge or otherwise deactivate radical species and reactive oxygen species (ROS) that are generated before, during or after imaging which comprises singlet oxygen, hydroxy radicals, superoxide anions and hydrogen peroxide. Such photon scavenging molecules include ascorbic acid (vitamin C), tocopherol (vitamin E), 1,3-diphenylisobenzofuran (DPBF), 9,10-anthracenediyl-bis(methylene)dimalonic acid (ABDA), and unsaturated molecules such as retinol, carotenoid and cumene. A photodamage inhibitor may comprise a molecule that is exogenous to or endogenous to a source of an analyte or other array component (e.g., an anchoring moiety, a macromolecule, a blocking reagent, a detectable probe, etc.).

As used herein, the term “probe” refers to a composition comprising an affinity agent and a detectable label. A probe can comprise a plurality of affinity agents. A probe can comprise a plurality of detectable labels. A probe may comprise a plurality of affinity agents, in which each affinity agent comprises a same binding specificity. A probe may comprise a plurality of affinity agents, two or more affinity agents comprise differing binding specificities. A probe can comprise a linking moiety or retaining component (e.g., a nucleic acid nanoparticle) that couples an affinity agent to a detectable label. A probe can comprise a linking moiety that couples a first affinity agent to a second affinity agent. A probe can comprise a linking moiety that couples a first detectable label to a second detectable label. A probe may comprise a nucleic acid linking moiety, such as a nucleic acid nanoparticle or a structured nucleic acid nanoparticle. A linking moiety may comprise a tunable structure that facilitates orientation of two or more affinity agents relative to each other. A probe may comprise one or more moieties that are configured to prevent orthogonal binding, such as polyethylene glycol molecules or single-stranded nucleic acids.

As used herein, the terms “protein” or “polypeptide” refer synonymously to a molecule comprising two or more amino acids joined by a peptide bond. A protein may also be referred to as a polypeptide, oligopeptide or peptide. A protein can be a naturally-occurring molecule, or synthetic molecule. A protein may include one or more non-natural amino acids, modified amino acids, or non-amino acid linkers. A protein may contain D-amino acid enantiomers, L-amino acid enantiomers or both. Amino acids of a protein may be modified naturally or synthetically, such as by post-translational modifications. In some circumstances, different proteins may be distinguished from each other based on different genes from which they are expressed in an organism, different primary sequence length or different primary sequence composition. Proteins expressed from the same gene may nonetheless be different proteoforms, for example, being distinguished based on non-identical length, non-identical amino acid sequence or non-identical post-translational modifications. Different proteins can be distinguished based on one or both of gene of origin and proteoform state.

As used herein, the term “single,” when used in reference to an object such as an analyte, means that the object is individually manipulated or distinguished from other objects. A single analyte can be a single molecule (e.g. single protein), a single complex of two or more molecules (e.g. a multimeric protein having two or more separable subunits, a single protein attached to a structured nucleic acid particle or a single protein attached to an affinity reagent), a single particle, or the like. Reference herein to a “single analyte” in the context of a composition, system or method herein does not necessarily exclude application of the composition, system or method to multiple single analytes that are manipulated or distinguished individually, unless indicated contextually or explicitly to the contrary.

As used herein, the term “single-analyte resolution” refers to the detection of, or ability to detect, an analyte on an individual basis, for example, as distinguished from its nearest neighbor in an array. Single-analyte resolution can refer to an analyte being detectable by detection of a signal associated with the analyte, and the signal associated with the analyte being resolvable from signals associated with neighboring analytes.

As used herein, the term “solid support” refers to a substrate that is insoluble in aqueous liquid. Optionally, the substrate can be rigid. The substrate can be non-porous or porous. The substrate can optionally be capable of taking up a liquid (e.g. due to porosity) but will typically, but not necessarily, be sufficiently rigid that the substrate does not swell substantially when taking up the liquid and does not contract substantially when the liquid is removed by drying. A nonporous solid support is generally impermeable to liquids or gases. Exemplary solid supports include, but are not limited to, glass and modified or functionalized glass, plastics (including acrylics, polystyrene and copolymers of styrene and other materials, polypropylene, polyethylene, polybutylene, polyurethanes, Teflon™, cyclic olefins, polyimides etc.), nylon, ceramics, resins, Zeonor™, silica or silica-based materials including silicon and modified silicon, carbon, metals, metal oxides, metal carbides, metal nitrides, metal sulfides, inorganic glasses, optical fiber bundles, gels, and polymers. In particular configurations, a solid support may comprise a first solid material disposed upon a second solid material. For example, a solid support may comprise a metal oxide layer disposed upon a silicon or glass material. In particular configurations, a flow cell contains the solid support such that fluids introduced to the flow cell can interact with a surface of the solid support to which one or more components of a binding event (or other reaction) is attached.

As used herein, the term “structured nucleic acid particle” or “SNAP” refers to a single- or multi-chain polynucleotide molecule having a compacted three-dimensional structure. The compacted three-dimensional structure can optionally be characterized in terms of hydrodynamic radius or Stoke's radius of the SNAP relative to a random coil or other non-structured state for a nucleic acid having the same sequence length as the SNAP. The compacted three-dimensional structure can optionally be characterized with regard to tertiary structure. For example, a SNAP can be configured to have an increased number of internal binding interactions between regions of a polynucleotide strand, less distance between the regions, increased number of bends in the strand, and/or more acute bends in the strand, as compared to a nucleic acid molecule of similar length in a random coil or other non-structured state. Alternatively or additionally, the compacted three-dimensional structure can optionally be characterized with regard to tertiary or quaternary structure. For example, a SNAP can be configured to have an increased number of interactions between polynucleotide strands or less distance between the strands, as compared to a nucleic acid molecule of similar length in a random coil or other non-structured state. In some configurations, the secondary structure of a SNAP can be configured to be more dense than a nucleic acid molecule of similar length in a random coil or other non-structured state. A SNAP may contain DNA, RNA, PNA, modified or non-natural nucleic acids, or combinations thereof. A SNAP may include a plurality of oligonucleotides that hybridize to form the SNAP structure. The plurality of oligonucleotides in a SNAP may include oligonucleotides that are attached to other molecules (e.g., probes, analytes such as proteins, reactive moieties, or detectable labels) or are configured to be attached to other molecules (e.g., by functional groups). A SNAP may include engineered or rationally designed structures. Exemplary SNAPs include nucleic acid origami and nucleic acid nanoballs.

As used herein, the term “substantially uniform,” when used in reference to a plurality of molecules, refers to the plurality of molecules as containing a vast majority (e.g., at least about 90%, 95%, 96%, 97%, 98%, 99%, 99.5%, 99.9%, 99.99%, 99.999%, or more than 99.999% on a molar or mass basis) of molecules with uniform structural or physical properties. A plurality of molecules may be substantially uniform with respect to a structural property, a physical property, or both a structural and a physical property. For example, a plurality of polyethylene glycol (PEG) molecules containing a molecular weight distribution may be considered substantially uniform with respect to chemical structure (i.e., repeating polymerized ethylene glycol monomers). In another example, a plurality of PEG molecules comprising a mixture of linear and branched molecular chains may be considered substantially uniform with respect to a physical property if all molecules of the mixture are configured to inhibit binding of a particular assay agent, as set forth herein.

### Methods of Inhibiting Photodamage

In an aspect, provided herein is a method, comprising: a) providing a single-analyte array comprising a plurality of sites, in which each individual site of the plurality of sites comprises one and only one macromolecule of a first plurality of macromolecules, b) binding a second plurality of macromolecules to macromolecules of the first plurality of macromolecules at a fraction of sites of the plurality of sites, thereby forming a macromolecule complex at each individual site of the fraction of sites, in which each individual macromolecule complex comprises a macromolecule of the second plurality of macromolecules bound to one and only one macromolecule of the first plurality of macromolecules, c) detecting in the presence of photons a signal at each individual site of the first fraction of sites, and d) after detecting in the presence of photons the signal at each individual site of the first fraction of sites, dissociating macromolecules of the second plurality of macromolecules from macromolecules of the first plurality of macromolecules, in which each individual macromolecule complex comprises a plurality of photodamage inhibitor moieties that are coupled to the individual macromolecule complex.

Methods set forth herein utilize the presence of photodamage inhibitors in a single-analyte array system, as set forth herein, to inhibit deleterious interactions of photons with components of the array system. In some cases, photodamage inhibitors may be coupled to a single-analyte array to provide fixed energy sinks for excess photons, or fixed reactive sinks for any photon-generated reactive species. In some cases, photodamage inhibitors may be uncoupled (e.g., suspended or solvated in a fluidic medium) but in contact with a single-analyte array or analytes attached thereto.

Photodamage inhibitors may be provided to a single-analyte array system, as set forth herein, as an incorporated photodamage inhibitor or an unincorporated photodamage inhibitor. An incorporated photodamage inhibitor can refer to any photodamage inhibitor that is attached to a particle, molecule, or moiety, in which the particle, molecule or moiety is provided to a single-analyte array system for a purpose other than preventing photodamage. For example, photodamage inhibitors may be incorporated into passivating molecules that bind surface defects (e.g., dextrans, PEG molecules, etc.) to reduce orthogonal binding. In another example, photodamage inhibitors may be incorporated into anchoring moieties that couple an analyte to an analyte binding site of a single-analyte array. In another example, photodamage inhibitors may be incorporated into detectable probes that bind to analytes at analyte binding sites of a single-analyte array. In another example, photodamage inhibitors may be incorporated into a macromolecule that remains solvated in solution (e.g., a polymer) and does not couple to a single-analyte array, in which the macromolecule is provided to the single-analyte array system as a photon sink. An unincorporated photodamage inhibitor can refer to any photodamage inhibitor that is not attached to a particle, molecule, or moiety, in which the particle, molecule or moiety is provided to a single-analyte array system for a purpose other than preventing photodamage. For example, a photodamage inhibitor may be a small molecule oxygen scavenger or radical scavenger that remains free in solution. In some cases, an unincorporated photodamage inhibitor can be attached to a molecule, particle, or moiety, thereby converting the unincorporated photodamage inhibitor into an incorporated photodamage inhibitor. For example, a photodamage inhibitor may be coupled to an intercalating molecule, thereby facilitating attachment of the photodamage inhibitor to nucleic acids.

Photodamage inhibitors may be coupled or uncoupled to a single-analyte array, as set forth herein. An uncoupled photodamage inhibitor can refer to a photodamage inhibitor that is not coupled or attached to a single-analyte array or a surface thereof but is nonetheless in fluidic communication with the single-analyte array or surface thereof. An uncoupled photodamage inhibitor may be solvated or suspended in a fluidic medium. An uncoupled photodamage inhibitor may be attached to a molecule, particle, or moiety, in which the photodamage inhibitor and the molecule, particle, or moiety are both not coupled or attached to a single-analyte array or a surface thereof. A coupled photodamage inhibitor can refer to a photodamage inhibitor that is coupled or attached to a single-analyte array or a surface thereof. A coupled photodamage inhibitor may be covalently or non-covalently coupled or attached to a single-analyte array or a surface thereof. A coupled photodamage inhibitor may be coupled or attached to a molecule, particle, or moiety that is coupled or attached to a single-analyte array or a surface thereof. Accordingly, a coupled photodamage inhibitor may be immobilized on a single-analyte array or a surface thereof via the molecule, particle, or moiety.

Methods set forth herein may involve one or more steps of introducing photodamage inhibitors into processes of single-analyte array systems before, during, or after a process step that involves exposing a single-analyte array to an electromagnetic radiation field (e.g., a light beam, a light field). Photodamage inhibitors may be provided to a single-analyte array before or during an exposure of a single-analyte array to an electromagnetic radiation field, thereby providing energy sinks or reactive sinks for inhibiting deleterious photon-related interactions. Photodamage inhibitors may be provided to a single-analyte array after an exposure of a single-analyte array to an electromagnetic radiation field, thereby replenishing available energy sinks or reactive sinks for subsequent exposure of the single-analyte array to the electromagnetic radiation field.

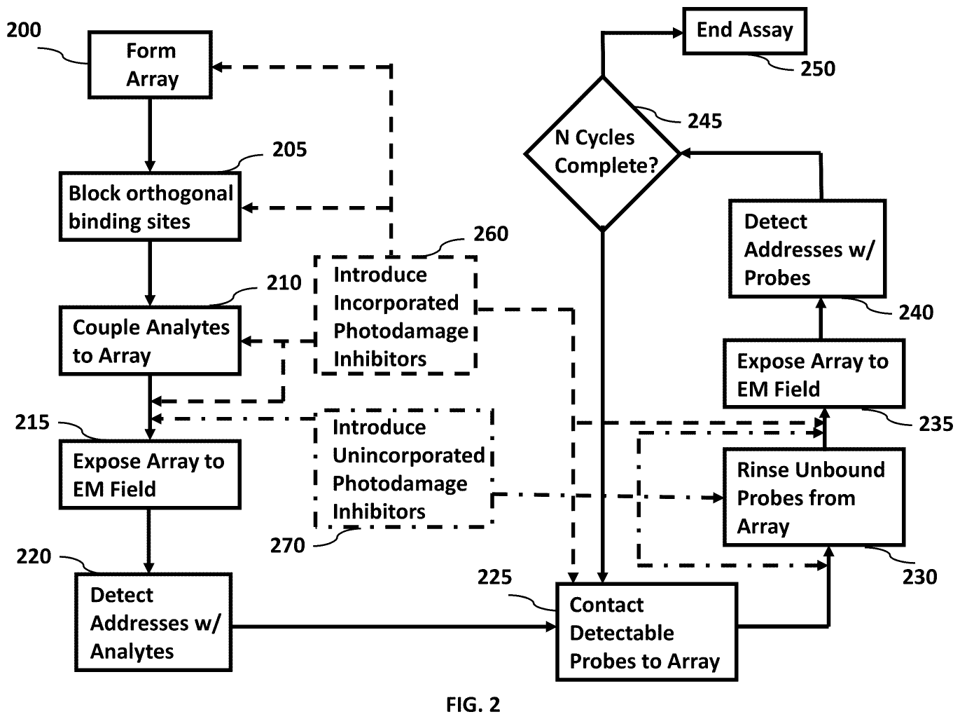

FIG. 2 illustrates a flow chart of a single-analyte array process involving formation of a single-analyte array followed by cyclical contacting of the analytes on the array with detectable probes. The method depicted in FIG. 2 includes multiple steps before, during, or after which photodamage inhibitors can be added to a single-analyte array system, as set forth herein. FIG. 2 exemplifies utilization of photodamage inhibitors during a cyclical, probe-based assay, but the skilled person can readily envision modification of the method for other cyclical processes (e.g., Edman-type degradation protein sequencing processes), or sequential processes. As shown in FIG. 2, a method may optionally begin with a first step 200 of forming a single-analyte array, as set forth herein. After forming an array, a subsequent step 205 may be to contact a surface of the array with a blocking agent that binds to defect sites on the surface, thereby inhibiting orthogonal binding to the array surface. Optionally, step 205 may occur after step 210, the deposition of single analytes at unique analyte binding sites of the single-analyte array. After depositing single analytes on the array, a subsequent optional step 215 may be to expose the array to an electromagnetic (EM) radiation field, thereby producing signals from array addresses comprising single analytes. Step 220 may involve detecting signals from array addresses at single-analyte resolution, thereby facilitating identification of array addresses comprising analytes. Steps 225-250 depict a cyclical method for characterizing arrays with N sets of detectable probes. Step 225 comprises contacting the array with a set of detectable probes, thereby coupling detectable probes to a subset of analytes on the array. Step 230 is an optional rinsing step to remove unbound detectable probes from the single-analyte array system. Subsequently, step 235 can comprise exposing the array to an electromagnetic (EM) radiation field, thereby producing signals from array addresses comprising detectable probes. In step 240, signals may be detected at a subset of array addresses at single-analyte resolution, thereby facilitating identification of array addresses comprising detectable probes coupled to analytes. Decision 245 is to determine if all N cycles have been completed (i.e., all N sets of detectable probes utilized). If not, step 225 is repeated with a new set of detectable probes. If all N cycles have been completed, the assay may be terminated in step 250.

FIG. 2 also depicts steps before, during, or after which photodamage inhibitors may be introduced to a single-analyte system. In some cases, photodamage inhibitors may be introduced at multiple steps of a method set forth herein. Step 260 comprises introducing incorporated photodamage inhibitors to a single-analyte array system. Step 260 may be performed before or during any method step that involves coupling matter to a single-analyte array. For example, photodamage inhibitors may be incorporated into a surface of a single-analyte array during array formation. In another example, photodamage inhibitors may be incorporated into anchoring moieties that facilitate deposition of analytes onto an array. Additionally, step 260 may be performed before or during method steps involving an EM field. For example, an array may be exposed to an EM field in the presence of a liquid-phase polymeric molecule comprising incorporated photodamage inhibitors. Step 270 comprises introducing unincorporated photodamage inhibitors to a single-analyte array system. Step 270 may be performed at any conceivable step, including before steps involving exposing a single-analyte array to an EM field. For example, a reactive radical or oxygen scavenger such as ascorbic acid (vitamin C), tocopherol (vitamin E), 1,6-bis-di-tert-butylphenol, hydroxyl amines, TEMPO (2,2,6,6-tetramethylpiperidine 1-oxyl, 2,2,6,6-tetramethyl-1-piperidinyloxy), 4-hydroxy-TEMPO, 4-amino-TEMPO, 1,3-diphenylisobenzofuran (DPBF), 9,10-anthracenediyl-bis(methylene)dimalonic acid (ABDA), ascorbic acid, polyunsaturated molecules such as retinol, carotenoid, or cumene, or sodium azide may be introduced to or maintained at a particular concentration before or during EM field exposure. Step 270 may also be performed during certain replenishment processes. For example, unincorporated photodamage inhibitors may be contacted with analyte anchoring moieties that are partially depleted of incorporated photodamage inhibitors, thereby facilitating incorporation of additional photodamage inhibitors into the analyte anchoring moieties.

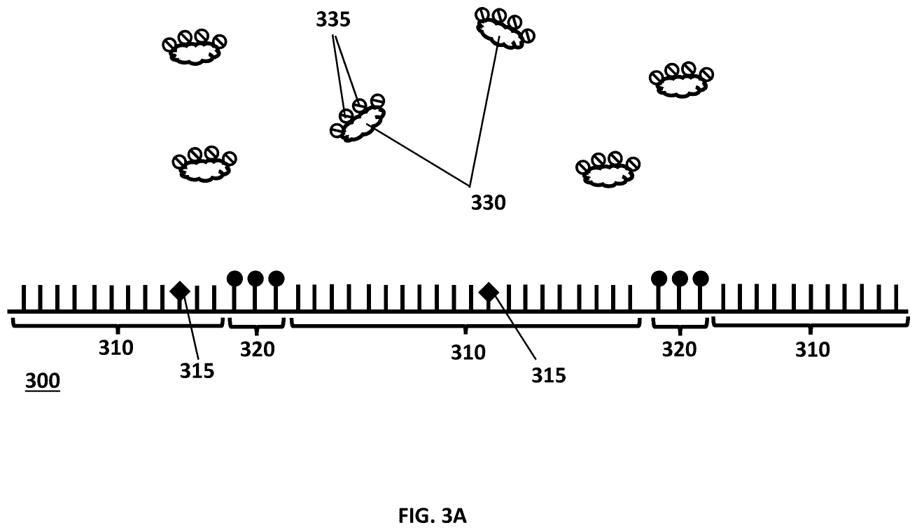

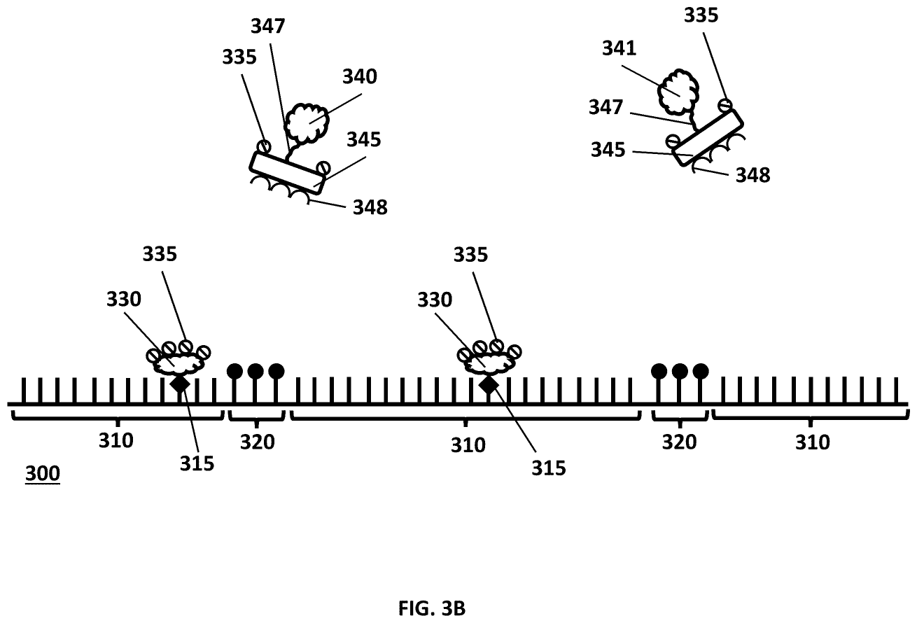

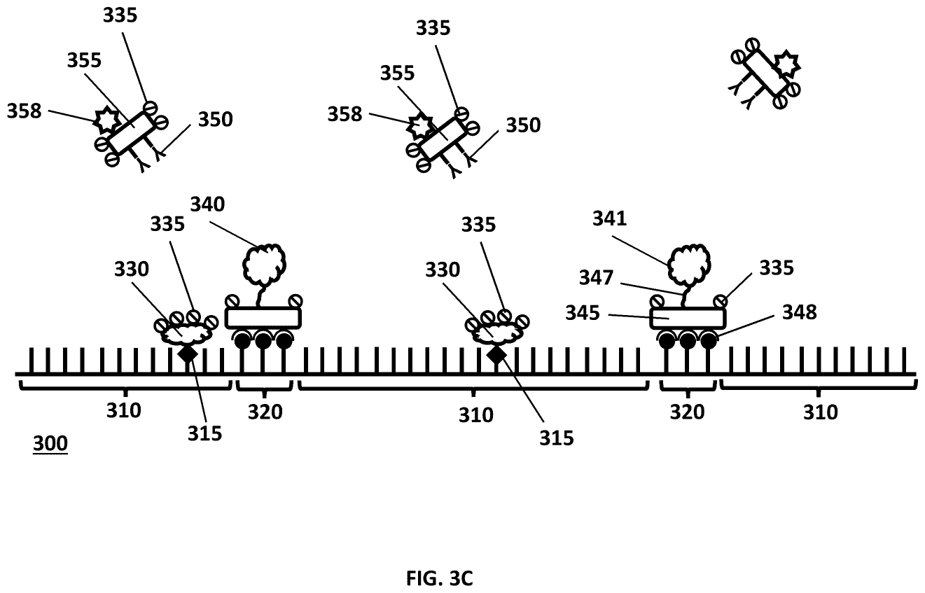

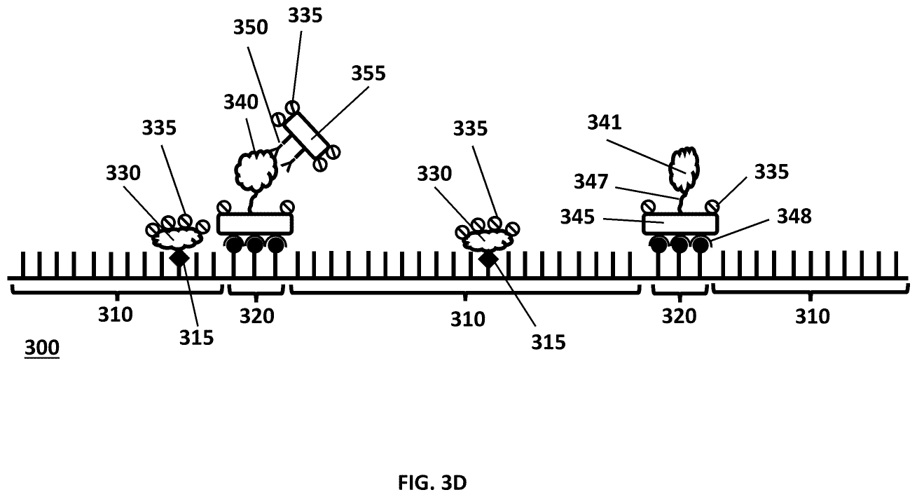

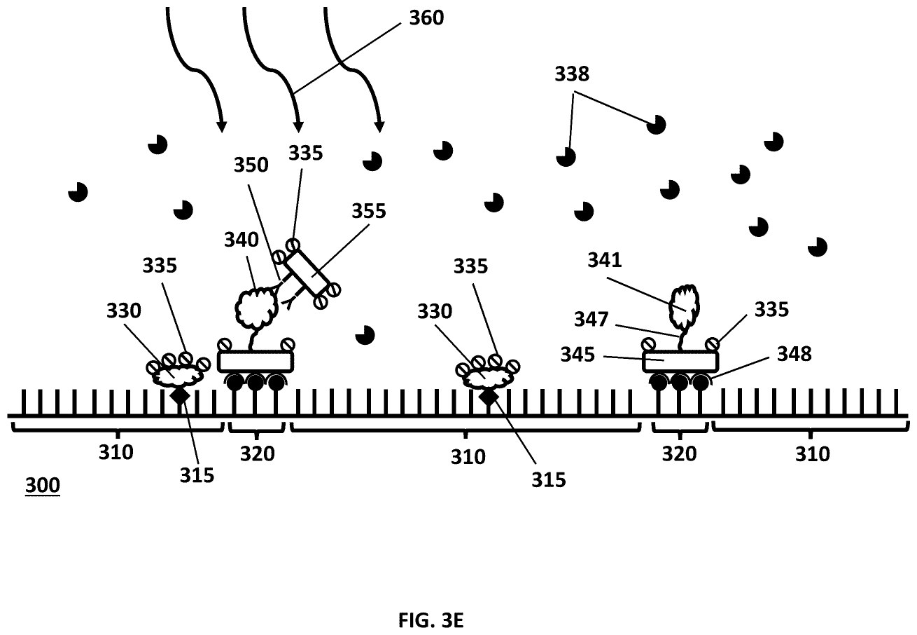

FIGS. 3A-3E depict a method of forming and utilizing a single-analyte array with incorporated photodamage inhibitors, in accordance with some embodiments. FIG. 3A illustrates an array composition comprising a solid support 300. The upper surface of the solid support 300 has been patterned to form analyte binding sites that are separated from other analyte binding sites by interstitial regions, in which analyte binding sites comprise pluralities of coupling moieties 320, and in which interstitial regions comprise pluralities of passivating moieties 310. The patterned surface of the solid support 300 may further comprise defects 315, in which defects are potential addresses for orthogonal binding (i.e., non-targeted binding). The array composition is contacted with a blocking agent comprising a plurality of macromolecular structures 330 (e.g., dextrans, PEG molecules, etc.), in which photodamage inhibitors 335, as set forth herein, have been attached to the macromolecular structures 330. FIG. 3B depicts the array composition after macromolecular structures 330 have coupled to defects 315, thereby reducing the likelihood of orthogonal binding to the array surface. Deposition of the macromolecular structures has coupled photodamage inhibitors to the array surface in a spatially random distribution that reflects the spatially random distribution of array defects 315. Accordingly, coupled photodamage inhibitors are located more closely to the left analyte binding site than the right analyte binding site. The array composition is contacted with a plurality of analytes (340 and 341, respectively). Each analyte 340 or 341 is coupled to an anchoring moiety 345 by a linking moiety 347 that couples the anchoring moiety 345 to the analyte 340 or 341. The anchoring moieties 345 further comprise complementary coupling moieties 348 that are configured to form binding interactions with coupling moieties 320 at analyte binding sites. The anchoring moieties further comprise photodamage inhibitors 335 that are incorporated with the anchoring moiety structures. The ordering of the blocking step of FIG. 3A and the analyte deposition step of FIG. 3B may be reversed, although it may be advantageous to block defects 315 first to prevent orthogonal binding of analytes 340 or 341 or anchoring moieties 345 to the defects 315. FIG. 3C depicts a formed single-analyte array, in which analytes 340 and 341 have been attached to differing analyte binding sites, and no other analytes are co-located at the analyte binding sites. Accordingly, binding of the anchoring moieties to the analyte binding sites therefore couples photodamage inhibitors 335 to analyte binding sites.

FIG. 3C further depicts contacting of the formed single-analyte array with a plurality of detectable probes. Each detectable probe comprises an affinity agent 350 (or optionally two or more affinity agents 350) and a detectable label 358 (or optionally two or more detectable labels 358). Optionally, the detectable probes may comprise a retaining component 355 (e.g., a polymeric nanoparticle, an inorganic nanoparticle, a nucleic acid nanoparticle, etc.) that couples together the at least one affinity agent 350 and the at least one detectable label 358. The detectable probe further comprises photodamage inhibitors 335. FIG. 3D illustrates an array configuration after an affinity agent 350 of a detectable probe has bound to analyte 340 at the left analyte binding site. Accordingly, coupling of the detectable probe to the analyte 340 couples additional photodamage inhibitors 335 at the address of the analyte binding site containing analyte 340. FIG. 3E depicts a detection step, in which the array composition is exposed to an electromagnetic (EM) radiation field 360. The exposing of the array to the electromagnetic radiation field (360) occurs in the presence of uncoupled photodamage inhibitors 338 (e.g., a reactive scavenger or a photon scavenger). The presence of incorporated photodamage inhibitors 335 and unincorporated photodamage inhibitors 338 may reduce the likelihood of photodamage to analytes 340 and 341 during one or more exposures to the EM field 360.

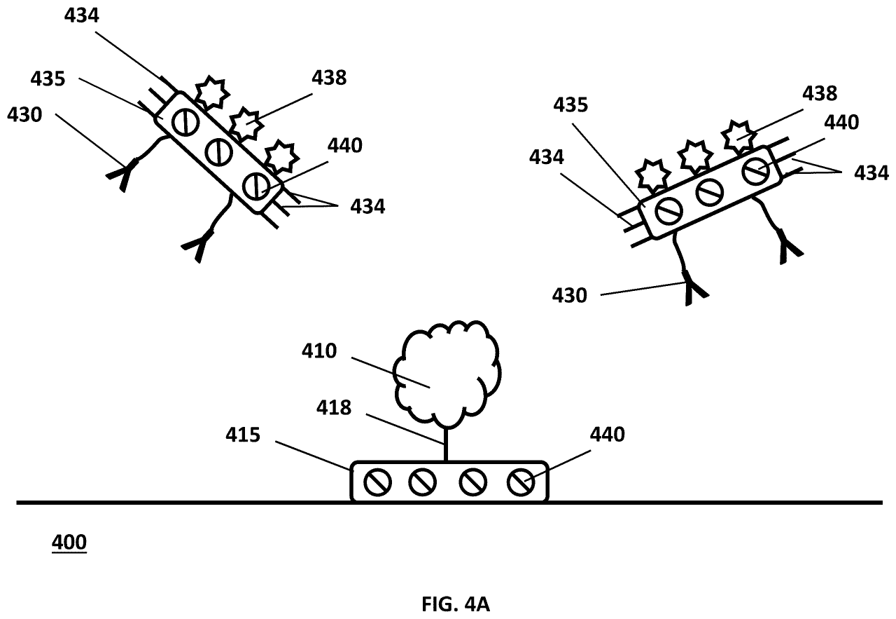

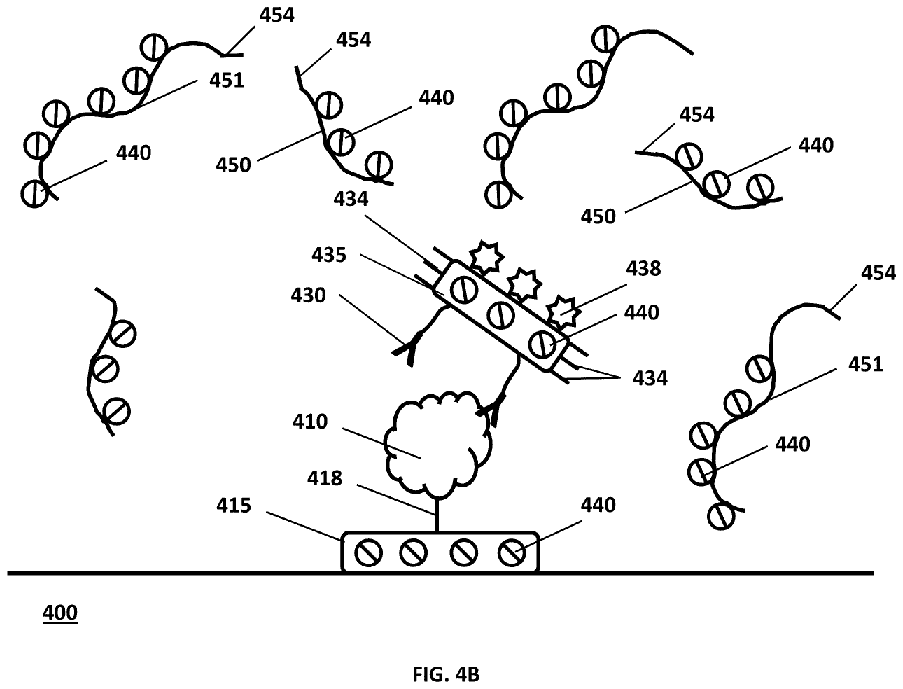

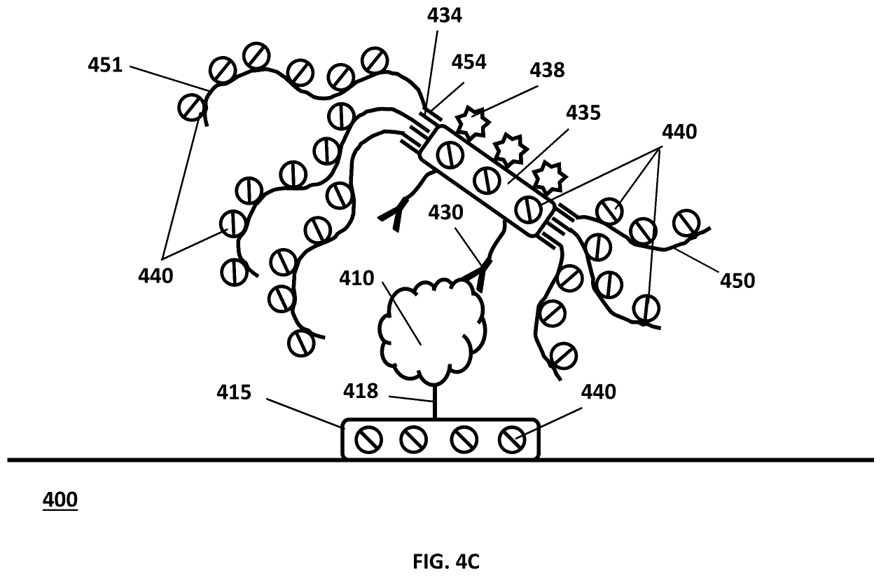

FIGS. 4A-4C depict a method of providing photodamage inhibitors to a surface-coupled analyte. FIG. 4A depicts an analyte 410 coupled to a surface of a solid support 400. The analyte 410 is attached to an anchoring moiety 415 by a linking moiety 418, in which the anchoring moiety 415 couples the analyte 410 to the surface of the solid support 400, and in which the anchoring moiety optionally contains photodamage inhibitors 440. The solid support 400 is contacted with a plurality of detectable probes. Each detectable probe comprises an affinity agent 430 (or optionally two or more affinity agents 430) and a detectable label 438 (or optionally two or more detectable labels 438). Optionally, the detectable probes may comprise a retaining component 435 (e.g., a polymeric nanoparticle, an inorganic nanoparticle, a nucleic acid nanoparticle, etc.) that couples together the at least one affinity agent 430 and the at least one detectable label 438. Optionally, the detectable probe further comprises photodamage inhibitors 440. Each detectable probe further comprises a plurality of coupling groups 434 (e.g., single-stranded nucleic acids, a component of a receptor-ligand binding pair, a reactive group, etc.). FIG. 4B depicts an affinity agent 430 of a detectable probe that is bound to the analyte 410, thereby co-locating the detectable probe and the analyte at the same address on the solid support 400. The analyte-probe complex is contacted with a plurality of uncoupled photodamage inhibitor structures. Each photodamage inhibitor structure comprises a linking moiety 450 or 451, a plurality of photodamage inhibitors 440, and a complementary coupling group 454. For example, the complementary coupling group 454 may be composed of an oligonucleotide that is configured to couple to a complementary oligonucleotide 434 by a hybridization interaction. In another example, the coupling group 454 may be composed of one or more DNA intercalation moieties (e.g., ethidium, 6,4′-diamidino-2-phenylindole (DAPI), propidiurn, proflavine or Adriamycin) that can intercalate into a nucleic acid structure (e.g., anchoring moiety 415, retaining component 435). In yet another example, the coupling group 454 may comprise certain molecular residues that can engage in non-covalent association with a nucleic acid, polypeptide, or probe surface through electrostatic binding, dipole-dipole interaction, hydrogen bond, van der Waals hydrophobic interaction or a combination thereof.

FIG. 4C depicts the analyte-probe complex after the plurality of photodamage inhibitor structures has bound to the detectable probe by a coupling of coupling groups 434 to complementary coupling groups 454, thereby providing pendant moieties comprising photodamage inhibitors to the detectable probe. The analyte is now surrounded by an increased density of photodamage inhibitors 440. A skilled person can readily envision alternatively or additionally providing pendant moieties comprising photodamage inhibitors to an anchoring moiety 415, as set forth herein.

In an aspect, provided herein is a method, comprising: a) providing an array comprising a plurality of sites, in which each site of the plurality of sites comprises one and only one sample polypeptide of a plurality of sample polypeptides, in which the array further comprises a plurality of photodamage inhibitors, and in which photodamage inhibitors of the plurality of photodamage inhibitors are coupled to the array, b) detecting each sample polypeptide of the plurality of sample polypeptides on the array at single-analyte resolution in the presence of an electromagnetic radiation field, in which detecting each sample polypeptide on the array further comprises contacting each site of the plurality of sites with a minimum radiative input from the electromagnetic radiation field of at least 1×10−6 Joules (J), c) after contacting the array with the minimum radiative input, detecting at least 90% of sample polypeptides of the plurality of sample polypeptides on the array at single-analyte resolution.

A single-analyte array may be formed by coupling a plurality of single analytes to a plurality of analyte binding sites of an array composition, as set forth herein. In some cases, a single-analyte array may be formed by coupling a plurality of single analytes to a plurality of analyte binding sites of an array composition, as set forth herein, in which the plurality of single analytes is derived from a sample (e.g., sample polypeptides, sample nucleic acids, sample polysaccharides, etc.). Coupling a plurality of analytes (e.g., polypeptides, sample polypeptides, nucleic acids, sample nucleic acids, polysaccharides, sample polysaccharides, etc.) to a plurality of sites of a single-analyte array may comprise the steps of: i) coupling a single analyte of the plurality of analytes to an anchoring moiety, as set forth herein, and ii) coupling the anchoring moiety to a site of the plurality of sites. In some cases, an anchoring moiety may be non-covalently coupled to a site of a plurality of sites. In other cases, an anchoring moiety may be covalently coupled to the site of the plurality of sites.

Analytes (e.g., polypeptides, nucleic acids, polysaccharides, metabolites, etc.) may be coupled or conjugated to one or more anchoring moieties. An anchoring moiety may comprise a particle that mediates or facilitates the binding of the analyte to a substrate or surface. An anchoring moiety may comprise a particle that couples an analyte to a solid support. An anchoring moiety may comprise a particle such as a nucleic acid particle, a polypeptide, a polymer, an inorganic nanoparticle, an organic nanoparticle, or a combination thereof. An anchoring moiety may interact with a surface by an interaction such as electrostatic adhesion, magnetic adhesion, covalent bonding, ionic bonding, hydrogen bonding, or coordinate bonding. An anchoring moiety may interact with a surface in a reversible fashion or an irreversible fashion.

An analyte (e.g. a sample polypeptide or standard polypeptide) from a plurality of analytes may be coupled or conjugated to an anchoring moiety by a reversible or irreversible interaction. An analyte of a plurality of analytes may be coupled to an anchoring moiety of a plurality of anchoring moieties by a covalent bond. In some configurations, an analyte of a plurality of analytes may be coupled to an anchoring moiety of a plurality of anchoring moieties by a click reaction or other covalent coupling chemistry exemplified elsewhere herein. An analyte of a plurality of analytes may be coupled to an anchoring moiety of a plurality of anchoring moieties by a non-covalent interaction. In some configurations, the non-covalent interaction may be an electrostatic interaction, magnetic interaction, a hydrogen bond, or a binding interaction. In some configurations, the non-covalent hydrogen bond interaction may comprise nucleic acid hybridization. In other configurations, the non-covalent binding interaction may comprise a receptor-ligand interaction or a receptor-small molecule interaction, such as streptavidin-biotin, SpyCatcher-SpyTag, SnoopCatcher-SnoopTag, FITC-anti-FITC antibody, or digoxigenin-anti-digoxigenin antibody or other non-covalent interaction exemplified elsewhere herein.

An anchoring moiety may comprise a macromolecule or particle that possesses a positive or negative overall surface charge density. An anchoring moiety may comprise a macromolecule or particle that possesses a positive or negative region of surface charge density. The surface charge density of an anchoring moiety may be the opposite charge of a surface that an analyte conjugate is to be deposited upon. The surface charge density of an anchoring moiety may be neutral. The surface charge density of an anchoring moiety may be uniform over the available surface area of the anchoring moiety. A uniform surface charge density may increase the speed and/or likelihood of the anchoring moiety depositing upon a surface or material. Regions of positive or negative surface charge density of an anchoring moiety may be localized to one or more regions of the anchoring moiety structure. Localized surface charge density on an anchoring moiety may cause an analyte conjugate containing the anchoring moiety to deposit on a surface or material with a uniform or controlled orientation. Surface charge density of an anchoring moiety or an analyte conjugate containing an anchoring moiety may be measured by a suitable method such as electrophoretic measurement of zeta potential. A surface charge density of an anchoring moiety or an analyte conjugate containing an anchoring moiety may be determined by experimental measurement, computational modeling, or a combination thereof.

Anchoring moieties may comprise one or more macromolecules. A suitable macromolecule for an anchoring moiety may include a macromolecule with a uniform or localized region of positive or negative surface charge density. A macromolecule in an anchoring moiety may possess a controlled or engineered structure, including a feature such as an analyte coupling or conjugation site, or a surface bonding site. An analyte coupling or conjugation site on an anchoring moiety may comprise a functional group configured to react with a functional group of a functionalized or unfunctionalized polypeptide, thereby forming a covalent bond between the anchoring moiety and the analyte. Suitable macromolecules may include nucleic acids, proteins, or polymers. A nucleic acid anchoring moiety may comprise a structured nucleic acid particle (SNAP) such as a DNA nanoball, DNA nanotube, or DNA origami. A polypeptide-based anchoring moiety may include an engineered or non-engineered polypeptide that has a tendency to deposit on a surface or material. A polypeptide for a polypeptide-based anchoring moiety may be prepared for conjugation to a polypeptide from a polypeptide fraction by methods similar to those described above. A polymer-based anchoring moiety may include ionic or non-ionic polymers.

In other configurations, an anchoring moiety may comprise a particle, such as a nanoparticle, that provides a plurality of attachment sites for two or more binding components, and optionally one or more label components. In some configurations, a particle may comprise a surface that is functionalized, can be functionalized, or is otherwise modifiable to provide attachment sites for analyte coupling. In some configurations, a particle may provide a template for a shell, surface coating, or surface layer (e.g., a surface coating comprising a polymer or hydrogel coating, a surface layer of functional groups) that contains or can be modified to contain attachment sites for detectable probe components. A surface coating may comprise a polymer, biopolymer, metal, or metal oxide. In some configurations, an anchoring moiety may effectively function as a label component (e.g., a fluorosphere or quantum dot). A particle for an anchoring moiety may comprise a surface coating or surface layer that comprises a surface electrical charge. The surface electrical charge may comprise a net positive charge or a net negative charge. An anchoring moiety may be formulated or modified to comprise a plurality of functional groups that are configured to couple to a solid support by a covalent or non-covalent interaction. In some configurations, the plurality of functional groups may comprise a functional group selected from the group consisting of an alkyl, alkenyl, alkynyl, phenyl, halide, hydroxyl, carbonyl, aldehyde, acyl halide, ester, carboxylate, carboxyl, carboalkoxy, methoxy, hydroperoxy, ether, hemiacetal, hemiketal, acetal, ketal, orthoester, epoxide, carboxylic anhydride, carboxamide, amine, ketimine, aldimine, imide, azide, azo, cyanate, isocyanate, nitrate, nitrile, isonitrile, nitrosoxy, nitro, nitroso, oxime, pyridyl, carbamate, sulfhydryl, sulfide, disulfide, sulfinyl, sulfonyl, sulfinom, sulfo, thiocyanate, isothiocyanate, carbonothioyl, thioester, thionoester, phosphino, phosphono, phosphonate, phosphate, borono, boronate, and a borinate. In some configurations, an anchoring moiety may be modified to comprise a functional group that is configured to undergo a click reaction. In other configurations, an anchoring moiety may be modified to comprise a functional group that is configured to undergo a chemical cross-linking or a photo-initiated cross-linking reaction.

An anchoring moiety may comprise a detectable label that permits detection of the anchoring moiety. A detectable label may comprise a fluorescent label, a luminescent label, a radiolabel, an enzymatic tag, or a nucleic acid label or barcode. An anchoring moiety may be conjugated with a detectable label. The conjugated detectable label may be conjugated by a covalent bond (e.g., a reactive dye) or a non-covalent interaction (e.g., hybridization of a nucleic acid tag, an intercalation dye). A detectable label may be used to quantify anchoring moieties in solution or detect anchoring moieties at individual locations on a substrate.

An anchoring moiety or a linkage between an anchoring moiety and an analyte may further comprise a linker. A linker may comprise a bifunctional, trifunctional, or polyfunctional linker. A bifunctional linker may comprise a homobifunctional linker or a heterobifunctional linker. A linker may include a reporting molecule that is released upon successful coupling of an anchoring moiety to an analyte. For example, a click-to-release strategy may be utilized to covalently couple a polypeptide comprising a click handle with an anchoring moiety comprising a second click handle (e.g., inverse-electron demand Diels Alder click-to-release).

An anchoring moiety may be configured to be coupled or conjugated to a functionalized or unfunctionalized analyte (e.g. sample polypeptide or standard polypeptide). Functional groups capable of rapidly forming covalent bonds with functionalized or unfunctionalized analytes may be of particular interest for the functionalization of anchoring moieties. In general, functional groups of interest will include most common species for bioconjugation. Such functional groups may include “click-type” reagents that are capable of forming highly specific products with complementary functional groups in a rapid and irreversible fashion. An anchoring moiety may comprise one or more sites for analyte coupling or conjugation. An anchoring moiety with more than one attachment site may be capable of coupling or conjugating more than one analyte. An anchoring moiety may comprise a fixed number of analyte attachment sites, such as about 1, 2, 3, 4, 5, 6, 7, 8, 9, 10, 20, 30, 40, 50, 75, 100, 200, 300, 400, 500, 1000, 5000, 10000, 50000, 100000, 500000, 1000000, or more than 1000000 attachment sites. An anchoring moiety may comprise a fixed number of analyte attachment sites, such as at least about 1, 2, 3, 4, 5, 6, 7, 8, 9, 10, 20, 30, 40, 50, 75, 100, 200, 300, 400, 500, 1000, 5000, 10000, 50000, 100000, 500000, 1000000, or more than 1000000 attachment sites. Alternatively or additionally, an anchoring moiety may comprise a fixed number of analyte attachment sites, such as no more than about 1000000, 500000, 100000, 50000, 10000, 5000, 1000, 500, 400, 300, 200, 100, 75, 50, 40, 30, 20, 10, 9, 8, 7, 6, 5, 4, 3, 2, or less than 2 attachment sites. Additional aspects of anchoring moieties, analyte conjugation, and anchoring moiety deposition can be found in US20220227890A1, which is herein incorporated by reference in its entirety.

A method of forming a single-analyte array may further comprise coupling a plurality of photodamage inhibitors to an array composition, as set forth herein. In some cases, coupling a plurality of photodamage inhibitors to an array may comprise coupling a first photodamage inhibitor to a site of the plurality of sites. For example, an anchoring moiety may comprise an incorporated photodamage inhibitor, whereby coupling of the anchoring moiety at a site couples the photodamage inhibitor to the array at the site. In some cases, coupling a plurality of photodamage inhibitors to an array may comprise coupling a second photodamage inhibitor to an interstitial region of the array. For example, a blocking agent (e.g., a macromolecular structure) may comprise a photodamage inhibitor, whereby coupling the blocking agent to an orthogonal binding site (e.g., a charged surface defect) of an interstitial region couples the photodamage inhibitor to the array at the orthogonal binding site. In some cases, coupling a plurality of photodamage inhibitors to an array may comprise coupling photodamage inhibitors only to the plurality of sites. In other cases, coupling a plurality of photodamage inhibitors to an array may comprise coupling photodamage inhibitors only to interstitial regions. In some cases, coupling a plurality of photodamage inhibitors to an array may comprise coupling photodamage inhibitors to sites of the plurality of sites and interstitial regions.

Photodamage inhibitors may be directly coupled to an address of an array, as set forth herein. In some cases, an unincorporated photodamage inhibitor may be directly coupled to an address of an array. A photodamage inhibitor may be coupled to an address comprising an array site. A photodamage inhibitor may be coupled to an address comprising an interstitial region. A photodamage inhibitor may be directly coupled to an address of an array by a covalent interaction (e.g., a Click-type reaction, an amide bond, a coordination bond, etc.). A photodamage inhibitor may be directly coupled to an address of an array by a non-covalent interaction (e.g., electrostatic adhesion, magnetic adhesion, receptor-ligand binding, nucleic acid hybridization, etc.).

Photodamage inhibitors may be coupled to an array composition, as set forth herein, at any conceivable time, including when forming a single-analyte array, before performing an assay with the single-analyte array, or during assay of the single-analyte array. It may be most advantageous to couple photodamage inhibitors to an array before the array is exposed to an electromagnetic radiation field. In some cases, providing a single-analyte array may comprise coupling analytes of a plurality of analytes to the single-analyte array before coupling photodamage inhibitors of a plurality of photodamage inhibitors to the array. In particular cases, providing a single-analyte array may comprise coupling a plurality of analytes to the single-analyte array before coupling a plurality of photodamage inhibitors to the array. In some cases, providing a single-analyte array may comprise coupling analytes of a plurality of analytes to the single-analyte array after coupling photodamage inhibitors of a plurality of photodamage inhibitors to the array. In particular cases, providing a single-analyte array may comprise coupling a plurality of analytes to the single-analyte array after coupling a plurality of photodamage inhibitors to the array. In some cases, providing a single-analyte array may comprise simultaneously coupling analytes of a plurality of analytes and photodamage inhibitors of a plurality of photodamage inhibitors to the single-analyte array. In particular cases, providing a single-analyte array may comprise simultaneously coupling a plurality of analytes and a plurality of photodamage inhibitors to the single-analyte array.

A single-analyte array may be formed by a method that includes one or more steps that comprise coupling photodamage inhibitors to the single-analyte array. For example, photodamage inhibitors may first be coupled to a single-analyte array when coupling a blocking agent to orthogonal binding sites of a single-analyte array, and subsequently coupled to the single-analyte array when coupling analytes to the array. In some cases, a method may comprise forming a single-analyte array comprising a plurality of photodamage inhibitors, in which a first subset of the plurality of photodamage inhibitors is coupled to the single-analyte array in a first method step, as set forth herein, and in which a second subset of the plurality of photodamage inhibitors is coupled to the single-analyte array in a second method step, as set forth herein. In other cases, a method may comprise forming a single-analyte array comprising a plurality of photodamage inhibitors, in which all photodamage inhibitors of the plurality of photodamage inhibitors are coupled to the single-analyte array during a single method step, as set forth herein.

Coupling a plurality of photodamage inhibitors to a single-analyte array may comprise contacting photodamage inhibitors of the plurality of photodamage inhibitors to the array. In some cases, contacting photodamage inhibitors of a plurality of photodamage inhibitors to a single-analyte array occurs in the presence of analytes of a plurality of analytes. In other cases, contacting photodamage inhibitors of the plurality of photodamage inhibitors to a single-analyte array occurs in an absence of analytes of a plurality of analytes. A method of forming a single-analyte array may comprise the steps of: i) coupling a particle, molecule, or moiety to the single-analyte array, ii) contacting photodamage inhibitors of a plurality of photodamage inhibitors to the single-analyte array comprising the particle, molecule, or moiety, and iii) coupling the photodamage inhibitors of the plurality of photodamage inhibitors to the particle, molecule, or moiety. For example, an analyte may be coupled to a site of a single-analyte array by an anchoring moiety, in which the anchoring moiety is contacted with photodamage inhibitors, thereby coupling photodamage inhibitors to the anchoring moiety. After contacting photodamage inhibitors to a single-analyte array, the photodamage inhibitors may be coupled to the array by a covalent interaction. After contacting photodamage inhibitors to a single-analyte array, the photodamage inhibitors may be coupled to the array by a non-covalent interaction.

A method may comprise at least one step of detecting analytes coupled to a single-analyte array at single-analyte resolution. Detecting each analyte of a plurality of analytes on a single-analyte array at single-analyte resolution may comprise the steps of: i) contacting a site of a plurality of sites with an electromagnetic radiation field, and ii) detecting an electromagnetic signal from the site of the plurality of sites, in which the electromagnetic signal corresponds to a presence of a sample polypeptide of the plurality of sample polypeptides. An electromagnetic signal may be produced by a moiety (e.g., a fluorescent moiety, a luminescent moiety) in response to an electromagnetic radiation field. In some cases, a method may comprise simultaneously contacting a subset of a plurality of sites with an electromagnetic radiation field. For example, a single-analyte array may be exposed to a two-dimensional light field or a line of light, in which multiple sites are simultaneously illuminated by the light field. In some cases, a method may comprise simultaneously detecting electromagnetic signals from sites of a plurality of sites. For example, if sites containing fluorescent moieties are simultaneously illuminated by an electromagnetic radiation field, electromagnetic signals may be simultaneously detected from the sites. For some single-analyte assays, it is advantageous to couple detectable labels to analytes or anchoring moieties, in which the detectable labels are configured to provide electromagnetic signals in response to an electromagnetic radiation field. Accordingly, presence of an electromagnetic signal at an array address provides evidence of the presence of an anchoring moiety and/or an analyte at the array address.

For some single-analyte assays, it is also advantageous to utilize detectable probes to identify presence or absence of analytes at array addresses at single-analyte resolution. Co-location at an array address of a first electromagnetic signal from an analyte or an anchoring moiety and a second electromagnetic signal from a detectable probe that binds an analyte may confirm the presence of the analyte at the array address. For example, if an anchoring moiety without a coupled analyte is deposited at an array site, an electromagnetic signal may be detected from the anchoring moiety, but a detectable probe would not be expected to co-locate at the array site due to the absence of an analyte. A method may comprise a step of detecting each analyte of a plurality of analytes on a single-analyte array at single-analyte resolution, in which detecting each analyte further comprises the steps of: i) contacting the single-analyte array with a first plurality of detectable probes, ii) coupling detectable probes of the first plurality of detectable probes to analytes of the plurality of analytes at a subset of sites of the plurality of sites, iii) contacting the detectable probes with an electromagnetic radiation field, and iv) at each site of the subset of the plurality of sites, detecting presence of an electromagnetic signal from a detectable probe of the detectable probes. In some cases, a method may further comprise, at each site of a subset of a plurality of sites, detecting a second electromagnetic signal from an anchoring moiety or an analyte.

It may be useful to provide an avidity component at an array site to facilitate controlled binding of detectable probes to analytes at the array site. An avidity component may comprise any suitable moiety or ligand that has one or more properties of: i) facilitating binding of a first detectable probe at the array site, in which the first detectable probe comprises a mobile avidity component that is configured to bind to an immobilized avidity component, ii) inhibiting binding of a second detectable probe at the array site, in which the second detectable probe does not comprise an avidity component that is configured to bind to the avidity component, and iii) facilitating retention of an affinity agent of the first detectable probe at the array site until the presence of the first detectable probe has been detected.

Table II presents pairs of complementary avidity components. An avidity component may be chosen from column A or B as an immobilized avidity component, and the complementary avidity component in the other column may be chosen as the mobile avidity component. An immobilized avidity component may be immobilized at an array site by covalent coupling to the array site (e.g., covalently coupled to a surface-coupled moiety of the array site), or by covalent coupling to an anchoring group or analyte attached to the array site. An immobilized avidity component may be immobilized at an array site by non-covalent coupling to the array site (e.g., non-covalently coupled to a surface-coupled moiety of the array site), or by non-covalent coupling to an anchoring group or analyte attached to the array site. In some cases, a non-covalently coupled immobilized avidity component may be configured to dissociate from an array site. For example, an immobilized avidity component may be dissociated from an array site by denaturation, change in pH, change in ionic strength, nucleic acid dehybridization, enzymatic cleavage, photocleavage, change in temperature, contact with a chemical denaturant, or any other suitable mechanism of disrupting the coupling of the immobilized avidity component to the array site. In some cases, after dissociating an immobilized avidity component from an array site, a second avidity component may be coupled to the array site.

TABLE II

| Avidity | Complementary |
|---|---|
| Component | Avidity Component |
| Oligonucleotide | Partially- or fully- |
| | complementary |
| | oligonucleotide |
| Oligonucleotide | Single-stranded DNA-binding |
| | protein (e.g., SSB, transcription |
| | promoters, transcription repressors) |
| Double-stranded | Double-stranded DNA-binding |
| nucleic acid | protein (e.g., histones) |
| Short peptide (e.g., | Short peptide-specific affinity |
| less than about 100, 75, | agent (e.g., antibody, aptamer, etc.) |
| 50, 25, 20, 15, 10, 5, 4, | |
| or 3 amino acids) | |
| Antibody | Antibody-binding protein (e.g., |
| | Protein A, Protein G, Protein L, |
| | Gamma Fc Receptor, etc.) |
| Enzymatic | Tethered enzymatic substrate |
| protein | (i.e., substrate covalently |
| | coupled to a linking moiety) |
| First complexing protein | Second complexing protein |
| (e.g., first procollagen) | (e.g., second procollagen) |
| Polyelectrolyte | Complementary |
| brush molecule | protein molecule |
| Chemical cross- | Complementary chemical |
| linking agent | cross-linking agent |
| Photo-catalyzed | Complementary photo-catalyzed |
| cross-linking | cross-linking agent |
| agent (e.g., thiol | |

A first array site may be distinguished from a second array site by the presence of a first immobilized avidity component at the first array site and a differing second immobilized avidity component at the second array site. Accordingly, a first detectable probe may be configured to bind to the first array site by comprising a complementary mobile avidity component to the first immobilized avidity component, and a second detectable probe may be configured to bind to the second array site by comprising a complementary mobile avidity component to the second immobilized avidity component. In some cases, a first immobilized avidity component may differ from a second immobilized avidity component with respect to type of avidity component (e.g., selected from different rows of Table II). For example, a first array site may comprise an immobilized polymer brush and a second array site may comprise an immobilized antibody-binding protein. In some cases, a first mobile avidity component may differ from a second mobile avidity component with respect to type of avidity component (e.g., selected from different rows of Table II). For example, a first detectable probe may comprise a protein that is bound by a polymer brush, and a second detectable probe may comprise an antibody that is bound by an antibody-binding protein. In some cases, a first immobilized avidity component and a second avidity component may be the same type of avidity component, but may differ with respect to a characteristic of the type of avidity component, such as a residue sequence (e.g., amino acid sequence, nucleotide sequence), a secondary or tertiary structure, a binding affinity, a binding specificity, or a combination thereof. For example, a first array site may comprise an immobilized oligonucleotide with a first nucleotide sequence and a second array site may comprise an immobilized oligonucleotide with a second nucleotide sequence.

Detectable probes comprising an affinity agent and a mobile avidity component may be designed to have an effective binding affinity, effective association rate (i.e., on-rate), and/or effective dissociation rate (i.e., off-rate). Selection of a suitable mobile avidity component to pair with a particular affinity agent will depend, at least in part, on the binding characteristics of the affinity agent. To inhibit unwanted detection events of a detectable probe (e.g., due solely to binding of the mobile avidity component to an immobilized avidity component in the absence of binding of the affinity agent to an analyte), it may be preferable to select a mobile avidity component with less binding affinity for its complementary immobilized avidity component relative to the binding affinity of the affinity agent for its analyte target. In some cases, it may be preferable to form a detectable probe comprising an affinity agent and a mobile avidity component, in which one or both of the association rate and dissociation rate of the avidity component with its binding partner are slower than one or both of the association rate and dissociation rate of the affinity agent with its binding partner (e.g., the mobile avidity component is slower to form a binding interaction and slower to dissociate from its binding interaction). In some cases, it may be preferable to form a detectable probe comprising an affinity agent and a mobile avidity component, in which one or both of the association rate and dissociation rate of the avidity component with its binding partner are faster than one or both of the association rate and dissociation rate of the affinity agent with its binding partner (e.g., the mobile avidity component is faster to form a binding interaction and faster to dissociate from its binding interaction).

For an array comprising two or more differing immobilized avidity components, a binding characteristic (e.g., binding affinity, association rate, dissociation rate) of a first immobilized avidity component may differ from (e.g., greater than, less than) a binding characteristic of a second immobilized avidity component. Likewise, for a plurality of detectable probes containing two or more mobile avidity components, a binding characteristic (e.g., binding affinity, association rate, dissociation rate) of a first immobilized avidity component may differ from (e.g., greater than, less than) a binding characteristic of a second immobilized avidity component. In some cases, a binding affinity of a first mobile avidity component for a first immobilized avidity component is weaker than a binding affinity of a first affinity agent for a first analyte. In some cases, a binding affinity of a second mobile avidity component for the second immobilized avidity component is weaker than a binding affinity of a second affinity agent for a second analyte. In some cases, a binding affinity of a first mobile avidity component for a first immobilized avidity component is stronger than a binding affinity of a second mobile avidity component for a first immobilized avidity component. In some cases, a binding affinity of a second mobile avidity component for a second immobilized avidity component is stronger than a binding affinity of a first mobile avidity component for a second immobilized avidity component.

Accordingly, a suitable avidity component may increase an effective binding on-rate for a detectable probe, decrease an effective binding off-rate of a detectable probe, or decrease an effective dissociation constant of a detectable probe. Without wishing to be bound by theory, an avidity component may facilitate retention of a bound detectable probe at an array site by increasing the overall strength of binding interactions that must be overcome to release the detectable probe from the array site.

An immobilized avidity component may be located at an array site. An immobilized avidity component may be covalently coupled to an array site. An immobilized avidity component may be non-covalently coupled to an array site. An immobilized avidity component may be co-located with an analyte at an array site. An immobilized avidity component may be co-located with an analyte at an array site by a covalent coupling of the immobilized avidity component to the analyte. An immobilized avidity component may be co-located with an analyte at an array site by a non-covalent coupling of the immobilized avidity component to the analyte. An immobilized avidity component may be co-located with an analyte at an array site by a covalent coupling of the Immobilized avidity component to an anchoring group that is coupled to the array site. An immobilized avidity component may be co-located with an analyte at an array site by a non-covalent coupling of the immobilized avidity component to an anchoring group that is coupled to the array site.

In some configurations, an avidity component, as set forth herein, may further comprise a photodamage inhibitor or a plurality thereof. In some cases, a first avidity component may be coupled to a second avidity component, in which the first avidity component or the second avidity component comprises a photodamage inhibitor. In some cases, a first avidity component may be coupled to a second avidity component, in which the first avidity component and the second avidity component each individually comprise a photodamage inhibitor. In some cases, a method may comprise the steps of: i) coupling a first avidity component to a second avidity component to form a coupled avidity component pair, and ii) coupling a photodamage inhibitor to the coupled avidity component pair. For example, after coupling a first oligonucleotide avidity component to a second oligonucleotide avidity component to form a double-stranded nucleic acid, a photodamage inhibitor or a plurality of photodamage inhibitors may be coupled to the double-stranded nucleic acid by intercalation of an intercalating molecule that is coupled to the photodamage inhibitor or plurality thereof.

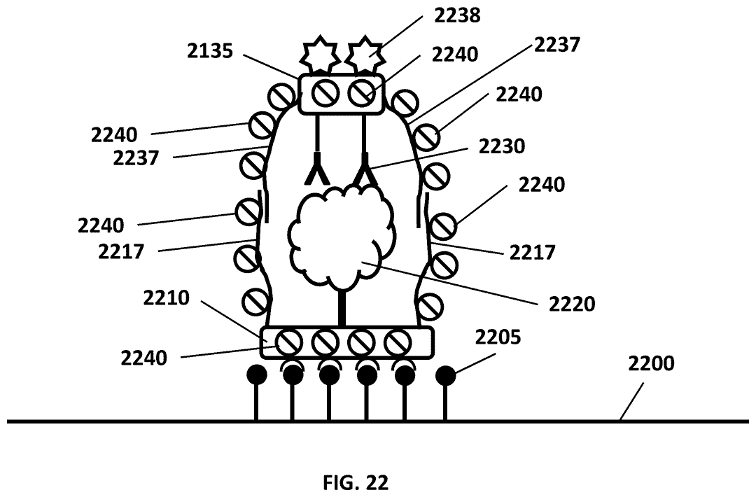

It may be advantageous to provide multiple pairs of avidity components at an array site. For example, an anchoring moiety may comprise two or more immobilized avidity components and a binding reagent may comprise two or more pairs of complementary mobile avidity components, in which each individual immobilized avidity component can form a binding interaction with an individual mobile avidity component. In some cases, an avidity component of a pair of avidity components may comprise one or more photodamage inhibitors. In some cases, each individual pair of avidity components may comprise photodamage inhibitors. A plurality of pairs of avidity components may provide an increased spatial concentration of photodamage inhibitors adjacent to an entity at an array site, such as an analyte, anchoring moiety, or binding reagent. FIG. 22 illustrates an array site composition containing a plurality of pairs of avidity components. A solid support 2200 contains an array site comprising a plurality of surface-coupled moieties 2205. The surface-coupled moieties 2205 form a binding interaction with an anchoring moiety 2210. An analyte 2220 is attached to the anchoring moiety 2210. The analyte 2220 is coupled to a binding reagent comprising an affinity agent 2230 (optionally a plurality of affinity agents 2230) and one or more detectable labels 2238. Optionally, a retaining moiety 2235 couples the affinity agent 2230 to the detectable label 2238. The binding reagent further comprises two or more avidity components 2237 that are coupled to complementary avidity components 2217 of the anchoring moiety 2210. A plurality of photodamage inhibitors 2240 are coupled at the array site, including photodamage inhibitors 2240 coupled to the retaining moiety 2210, the complementary avidity components 2217, the avidity components 2237, and the retaining moiety 2235. The plurality of photodamage inhibitors 2240 may surround the analyte 2220 and affinity agent 2230, possibly providing increased protection from photon-mediated deleterious interactions regardless of the incident direction of impinging light.

The skilled person will recognize that if two or more pairs of avidity components are provided to a macromolecular complex at an array site, it may be necessary to decrease the overall binding strength of the two or more pairs of avidity components (e.g., fewer base pairs of complementarity for oligonucleotide pairs, adding mispaired nucleotides to complementary oligonucleotide sequences, an antibody with a weaker antibody-binding protein, etc.). Too much avidity component pair binding strength may produce false positive detection (e.g., binding of a binding reagent at an array site due to avidity component binding rather than analyte-affinity agent binding) and/or inhibit dissociation of a binding reagent after a detection step.

Co-location of an anchoring moiety and/or an analyte with a detectable probe may be determined by detecting at an array address a first electromagnetic signal corresponding to the anchoring moiety and/or analyte, and a second electromagnetic signal corresponding to a detectable probe. A first electromagnetic signal corresponding to an anchoring moiety and/or analyte may be produced by a first detectable label (e.g., a fluorophore or a luminophore), and a second electromagnetic signal may be produced by a second detectable label. It may be advantageous to provide a first detectable label to an anchoring moiety and/or analyte, and a second detectable label to a detectable probe, in which the first detectable label has a differing excitation and/or emission wavelength from the second detectable label. Accordingly, a single-analyte array may be illuminated by an electromagnetic radiation field, in which the electromagnetic radiation field comprises light of a first wavelength and light of a second wavelength, in which a detectable probe of the detectable probes is configured to produce the electromagnetic signal in the presence of the light of the first wavelength, and in which the anchoring moiety and/or analyte is configured to produce the second electromagnetic signal in the presence of the light of the second wavelength.

Alternatively, a method may comprise a first step of illuminating a single-analyte array with a first electromagnetic radiation field comprising light of substantially a first single wavelength, and a second step of illuminating the single-analyte array with a second electromagnetic radiation field comprising light of substantially a second single wavelength. Illumination in a method set forth herein can deliver radiation in a particular region of the electromagnetic spectrum. For example, an array or other sample can be excited with radiation in the UV range of the spectrum (10 nm to 400 nm). In some cases, it may be desirable to use radiation in different sub-regions of the UV including, for example, the long wavelength ultraviolet (UVA) range (315 nm-400 nm), medium wavelength ultraviolet (UVB) range (280-315), or short wavelength ultraviolet (UVC) range (200-280 nm). Radiation in the longer wavelengths, for example, in the visible (VIS) or near infrared (NIR) regions of the spectrum can be used if desired. Optionally, radiation can be delivered in a particular region of the visible spectrum such as wavelengths at, below or above the red, orange, yellow, green, blue, or violet regions of the visible spectrum.

A single-analyte array method may be characterized based upon a radiative input provided to a site of a plurality of sites of a single-analyte array. A radiative input may be determined as a cumulative energy input provided by an electromagnetic field to a site of a single-analyte array. A radiative input may be calculated as:

$$R = P * {A}_{s} * {t}_{tot}$$

where the radiative input is a product of a power density, P, of an electromagnetic radiation field, an area of an array site, As, and a total exposure time of the site to the electromagnetic radiation field, tₜₒₜ. Presence of photodamage inhibitors at or adjacent to an array site comprising an analyte may decrease the likelihood of photodamage to the analyte for a given minimum radiative input or total exposure time. Alternatively, presence of photodamage inhibitors at or adjacent to an array site comprising an analyte may increase the average radiative input or total exposure time that an analyte can be subjected to before photodamage occurs.

In some cases, a site of a single-analyte array may be contacted with a first electromagnetic field comprising light of a first wavelength, and a second electromagnetic field comprising light of a second wavelength. A method may comprise providing at least a minimum radiative input to a site of a single-analyte array, in which the minimum radiative input comprises a first radiative input from light of a first wavelength and a second radiative input from light of a second wavelength. Given differences in photon energy based upon wavelength and differences in power density from light sources, an array site may receive differing radiative inputs from differing electromagnetic radiation fields. A minimum radiative input may comprise a a first radiative input from light of a first wavelength and a second radiative input from light of a second wavelength, in which the first radiative input comprises at least about 50%, 55%, 60%, 65%, 70%, 75%, 80%, 85%, 90%, 95%, 99%, 99.9%, or more than 99.9% of the minimum radiative input. Alternatively or additionally, a minimum radiative input may comprise a a first radiative input from light of a first wavelength and a second radiative input from light of a second wavelength, in which the first radiative input comprises no more than about 99.9%, 99%, 95%, 90%, 85%, 80%, 75%, 70%, 65%, 60%, 55%, 50%, or less than 50% of the minimum radiative input.

A method may comprise cyclical or sequential binding of detectable probes to analytes at array sites of a single-analyte array. Accordingly, a method may require repeated steps of binding detectable probes to analyte, and subsequently separating bound detectable probes from analytes. A method may comprise a step of separating detectable probes from analytes (e.g., sample polypeptides) at sites of the subset of the plurality of sites. Separating detectable probes from analytes may comprise contacting a single-analyte array with a probe dissociation medium. A probe dissociation medium may comprise a chaotropic agent that is configured to dissociate a binding interaction between a detectable probe and an analyte. Chaotropic agents are well known in the art, and may include but are not limited to sodium dodecyl sulfate, guanidinium chloride, magnesium chloride, lithium chloride, sodium hydroxide, hydrochloric acid, sodium thiocyanate, and sodium iodide.

A method may comprise separating bound detectable probes from analytes of a single-analyte array. A method may comprise separating detectable probes from sample polypeptides from a minimum quantity of sites of a subset of a plurality of sites, such as at least 50%, 60%, 70%, 80%, 90%, 95%, 99%, 99.9%, 99.99%, 99.999%, or more than 99.999% of sites of the subset of the plurality of sites, in which the subset comprises the sites to which the detectable probes are bound. A method may comprise the steps of: v) contacting a single-analyte array with an electromagnetic radiation field, and vi) detecting at a minimum quantity of sites of a subset of a plurality of sites an absence of an electromagnetic signal from a detectable probe of the detectable probes. For a cyclical or sequential single-analyte process, a method may further comprise, after separating detectable probes from analytes, performing one or more times the steps of: ii) coupling detectable probes of a plurality of detectable probes to sample polypeptides of the plurality of sample polypeptides at a subset of sites of the plurality of sites, iii) contacting the detectable probes with the electromagnetic radiation field, and iv) at each site of a subset of the plurality of sites, detecting presence of an electromagnetic signal from a detectable probe of the detectable probes. In some cases, a method may comprise contacting a single-analyte array with the first plurality of detectable probes (i.e., repeating a binding step with a same type of detectable probe). In other cases, a method may comprise contacting a single-analyte array with a second plurality of detectable probes, in which a first plurality of detectable probes comprises affinity for a differing set of analytes than the second plurality of detectable probes. A process of contacting analytes with detectable probes, detecting binding via irradiation, and/or separating detectable probes from analytes may be repeated at least 1, 2, 5, 10, 15, 20, 25, 30, 40, 50, 60, 75, 100, 125, 150, 200, 300, 400, 500, 1000, or more than 1000 times. Alternatively or additionally, a process of contacting analytes with detectable probes, detecting binding via irradiation, and/or separating detectable probes from analytes may be repeated no more than 1000, 500, 400, 300, 200, 150, 125, 100, 75, 60, 50, 40, 30, 25, 20, 15, 10, 5, 2, or less than 2 times. A method of providing photodamage inhibitors to a single-analyte array, as set forth herein, may facilitate a larger number of cycles of a single-analyte process due to a reduction in a rate of photodamage to analytes.

A single-analyte process may be characterized as providing a minimum radiative input to each site of a plurality of sites on a single-analyte array, in which the minimum radiative input increases the likelihood of photodamage to an analyte at an array site. In some cases, the minimum radiative input may be provided to a site of a plurality of sites during a single irradiation step (i.e., during a single, uninterrupted irradiation process). In other cases, contacting each site of a plurality of sites with a minimum radiative input from an electromagnetic radiation field may comprise two or more cycles or sequences of contacting each site of the plurality of sites with the electromagnetic radiation field, in which a radiative input to each site of the plurality of sites during each cycle of the two or more cycles is less than the minimum radiative input. For example, a ten cycle single-analyte process may comprise contacting each site of a plurality of sites with about one-tenth of the minimum radiative input per cycle, thereby providing about the minimum radiative input upon completion of all ten cycles. A method may further comprise, during each cycle or sequence of two or more cycles or sequences, detecting presence or absence of an electromagnetic signal at each site of the plurality of sites.

An analyte binding site may receive a radiative input of energy when exposed to an electromagnetic radiation field. A radiative input for an analyte binding site may be determined as the integrated value of all radiative energy contacted with the full surface area of an analyte binding site during exposure by an electromagnetic radiation field. In some cases, a radiative input of energy to an analyte binding site may comprise a minimum radiative input, as set forth herein, or a fraction thereof. In other cases, a radiative input of energy to an analyte binding site may not comprise a minimum radiative input. In some cases, a minimum radiative input may refer to a quantity of radiative input provided to an analyte binding site during a single-analyte assay or process, in which an analyte coupled to the analyte binding site has a threshold likelihood of being detectable at the completion of the assay or process (e.g., 50%, 60%, 70%, 80%, 90%, 95%, 99%, 99.9%, or greater than 99.9% chance of detectability after N cycles or steps). A minimum radiative input may be a function of detection system design, including choice of detectable label, power and/or power density of an excitation source (laser, lamp, LED, etc.), wavelength of an excitation source, illumination time per cycle or step, total number of cycles or steps in the assay or process, and quantity of available photodamage inhibitors coupled to or contacted with the array.

During a cycle or step of a single-analyte assay or process, an analyte binding site may receive a radiative input of at least about 1×10⁻⁹ Joules (J), 1×10⁻⁸ J, 1×10⁻⁷ J, 1×10⁻⁶ J, 5×10⁻⁶ J, 1×10⁻⁵ J, 5×10⁻⁵ J, 1×10⁻⁴ J, 1×10⁻³ J, or more than 1×10⁻³ J. Alternatively or additionally, during a cycle or step of a single-analyte assay or process, an analyte binding site may receive a radiative input of no more than about 1×10⁻³ J, 1×10⁻⁴ J, 5×10⁻⁵ J, 1×10⁻⁵ J, 5×10⁻⁶ J, 1×10⁻⁶ J, 1×10⁻⁷ J, 1×10⁻⁸ J, 1×10⁻⁹ J, or less than 1×10⁻⁹ J. During a single-analyte assay or process, an analyte binding site may receive a minimum radiative input of at least about 1×10⁻⁹ Joules (J), 1×10⁻⁸ J, 1×10⁻⁷ J, 1×10⁻⁶ J, 5×10⁻⁶ J, 1×10⁻⁵ J, 5×10⁻⁵ J, 1×10⁻⁴ J, 1×10⁻³ J, 1×10⁻²J, 1×10⁻¹ J, 1 J, or more than 1 J. Alternatively or additionally, during a single-analyte assay or process, an analyte binding site may receive a minimum radiative input of no more than about 1 J, 1×10⁻¹ J, 1×10⁻²J, 1×10⁻³ J, 1×10⁻⁴ J, 5×10⁻⁵ J, 1×10⁻⁵ J, 5×10⁻⁶ J, 1×10⁻⁶ J, 1×10⁻⁷ J, 1×10⁻⁸ J, 1×10⁻⁹ J, or less than 1×10⁻⁹ J.

During a cycle or step of a single-analyte assay or process, an analyte binding site may receive a radiative input for an illumination time of at least about 0.000001 seconds (s), 0.00001 s, 0.0001 s, 0.001 s, 0.005 s, 0.010 s, 0.015 s, 0.02 s, 0.025 s, 0.03 s, 0.04 s, 0.05 s, 0.1 s, 0.5 s, 1 s, 5 s, 10 s, 15 s, 30 s, 60 s, or more than 60 s. Alternatively or additionally, during a cycle or step of a single-analyte assay or process, an analyte binding site may receive a radiative input for an illumination time of no more than about 60 s, 30 s, 15 s, 10 s, 5 s, 1 s, 0.5 s, 0.1 s, 0.05 s, 0.04 s, 0.03 s, 0.025 s, 0.02 s, 0.015 s, 0.010 s, 0.005 s, 0.001 s, 0.0001 s, 0.00001 s, 0.000001 s, or less than 0.000001 s. During a single-analyte assay or process, an analyte binding site may receive a cumulative radiative input for an illumination time of at least about 0.00001 s, 0.0001 s, 0.001 s, 0.005 s, 0.010 s, 0.015 s, 0.02 s, 0.025 s, 0.03 s, 0.04 s, 0.05 s, 0.1 s, 0.5 s, 1 s, 5 s, 10 s, 15 s, 30 s, 60 s, 120 s, 300 s, 600 s, or more than 600 s. Alternatively or additionally, during a single-analyte assay or process, an analyte binding site may receive a cumulative radiative input for an illumination time of no more than about 600 s, 300 s, 120 s, 60 s, 30 s, 15 s, 10 s, 5 s, 1 s, 0.5 s, 0.1 s, 0.05 s, 0.04 s, 0.03 s, 0.025 s, 0.02 s, 0.015 s, 0.010 s, 0.005 s, 0.001 s, 0.0001 s, 0.00001 s, or less than 0.00001 s. During a single-analyte assay or process, an analyte binding site may receive a cumulative radiative input of at least about 1×10⁻⁹ Joules (J), 1×10⁻⁸ J, 1×10⁻⁷ J, 1×10⁻⁶ J, 5×10⁻⁶ J, 1×10⁻⁵ J, 5×10⁻⁵ J, 1×10⁻⁴ J, 1×10⁻³ J, 1×10⁻² J, 1×10⁻¹ J, 1 J, 10 J, 100 J, 1000 J, or more than 1000 J. Alternatively or additionally, during a single-analyte assay or process, an analyte binding site may receive a cumulative radiative input of no more than about 1000 J, 100 J, 10 J, 1 J, 1×10⁻¹ J, 1×10⁻² J, 1×10⁻³ J, 1×10⁻⁴ J, 5×10⁻⁵ J, 1×10⁻⁵ J, 5×10⁻⁶ J, 1×10⁻⁶ J, 1×10⁻⁷ J, 1×10⁻⁸ J, 1×10⁻⁹ J, or less than 1×10⁻⁹ J. During a single-analyte process, single analyte may be detected at array sites by localization of detectable probes at the array sites. A single-analyte process may comprise detecting a minimum percentage of single analytes of a plurality of analytes on a single-analyte array, such as at least about 1%, 5%, 10%, 20%, 30%, 40%, 50%, 55%, 60%, 65%, 70%, 75%, 80%, 85%, 90%, 95%, 99%, 99.9%, 99.99%, 99.999%, or more than 99.999% of single analytes on a single analyte array. Detecting at least a minimum percentage of single analytes of a plurality of analytes on an array may comprise detecting presence of an electromagnetic signal from a detectable probe from at least the minimum percentage of sites of the plurality of sites. For example, to detect the presence of at least 50% of single analytes on an array, electromagnetic signals from localized detectable probes need to be detected from at least 50% of sites of the array. Cycling or sequencing of detectable probe binding to single analytes may be utilized to increase the confidence of any single measurement. Accordingly, an electromagnetic signal from a localized detectable probe at an array site may be detected at least about 2, 3, 4, 5, 10, 15, 20, 25, 50, 100, or more than 100 times to detect the presence of an analyte at the array site. Alternatively or additionally, an an electromagnetic signal from a localized detectable probe at an array site may need to be detected no more than about 100, 50, 25, 20, 15, 10, 5, 4, 3, or less than 3 times to detect the presence of an analyte at an array site.

A method may comprise rinsing at least a fraction of the plurality of photodamage inhibitors from the array. Rinsing may comprise flowing a fluidic medium across a single-analyte array, or contacting a fluidic medium with the fluidic medium then draining or decanting the fluidic medium from the single-analyte array. In some cases, a rinsing fluidic medium may comprise photodamage inhibitors, as set forth herein. In other cases, a rinsing fluidic medium may lack photodamage inhibitors. Photodamage inhibitors may be rinsed from an array due to passive dissociation, disruption, or cleavage. Photodamage inhibitors may dissociate or be cleaved from an array due to irradiation by an electromagnetic field. For example, photolabile moieties may be cleaved from an array component comprising a plurality of photolabile moieties due to absorption of photons from a light field during an array process. Accordingly, photodamage inhibitors may be rinsed from a single-analyte array incidentally during any number of rinsing processes that may occur during a single-analyte process. In some cases, photodamage inhibitors may be actively removed by dissociation. For example, photodamage inhibitors coupled to detectable probes may be dissociated from an array due to a probe dissociation step that utilizes a probe dissociation medium, as set forth herein. In another example, blocking reagents comprising photodamage inhibitors may be stripped from array surfaces (e.g., interstitial regions, array sites) at some set interval (e.g., every N cycles) to remove depleted photodamage inhibitors and replenish them with fresh blocking reagents comprising photodamage inhibitors. A method may further comprise contacting a single-analyte array with a second plurality of photodamage inhibitors. In some cases, a method may further comprise coupling at least a fraction of the second plurality of photodamage inhibitors to a single-analyte array. For example, photodamage inhibitors may be coupled to blocking reagents (e.g., macromolecular structures) that are introduced during each cycle of a single-analyte process, of which a fraction become bound to unoccupied orthogonal binding sites during each cycle. For example, photodamage inhibitors (e.g., small molecule compounds, diffuse polymers with attached photodamage inhibitors) may be introduced and remain in a solution phase throughout a step of a single-analyte assay or process. In some cases, contacting of a second plurality of photodamage inhibitors may occur before contacting each site of a plurality of sites with a minimum radiative input from an electromagnetic radiation field. In other cases, contacting of a second plurality of photodamage inhibitors may occur during or after contacting each site of a plurality of sites with a minimum radiative input from an electromagnetic radiation field. In some cases, a method may further comprise coupling no photodamage inhibitors of the plurality of photodamage inhibitors to the array. For example, a step of a single-analyte assay or process may comprise contacting an array with a blocking reagent (e.g., a macromolecular structure) that comprises no photodamage inhibitors. In some cases, rinsing photodamage inhibitors of a first plurality of photodamage inhibitors from an array and coupling photodamage inhibitors of a second plurality of photodamage inhibitors to the array may occur simultaneously. For example, a rinsing medium may further comprise a blocking reagent (e.g., macromolecular structures comprising photodamage inhibitors), in which moieties of the blocking reagent are coupled to sites that are unoccupied due to dissociation of prior-coupled blocking reagent moieties containing photodamage inhibitors.

In another aspect, provided herein is a method, the method comprising performing at least two cycles of an assay, in which each cycle of the assay comprises the steps of: a) coupling a plurality of photodamage inhibitors to an array, in which the array comprises a plurality of sites, in which each site of the plurality of sites comprises one and only one analyte of a plurality of analytes (e.g., sample polypeptide, sample nucleic acids, sample polysaccharides, etc.), and in which each site of the plurality of sites is resolvable at single-analyte resolution, b) coupling detectable probes to sample polypeptides of the plurality of sample polypeptides, in which each probe of the detectable probes produces a detectable signal in the presence of an electromagnetic radiation field, and in which each site of the plurality of sites receives a minimum radiative input per cycle of at least 1×10⁻⁶ J, c) detecting presence or absence of the detectable signal from a probe of the detectable probes at each site of the plurality of sites, in which detecting the presence or absence of the probe comprises providing the electromagnetic radiation field, and d) after providing the electromagnetic radiation field, rinsing at least a fraction of the plurality of photodamage inhibitors from the array.

Accumulation of photodamage at array sites is increasingly likely to cause loss of detectability from a single analyte at an analyte binding site of a single-analyte array due to one or more of: 1) removal or destruction of the analyte, 2) chemical alteration of the analyte that renders detectable probes unable to bind (e.g., cross-linking of an affinity agent to a polypeptide; structural modifications at specific amino acid residues targeted by an affinity agent), 3) removal of the analyte from an anchoring moiety that couples the analyte to the analyte binding site, 4) removal of an anchoring moiety that couples the analyte to the analyte binding site, or 5) removal of a surface-coupled molecule or moiety that couples an anchoring moiety or analyte to the analyte binding site. Presence of a single analyte at an analyte binding site and its associated detectability cannot be proven through an absence of a signal associated with the analyte (e.g., from a bound detectable probe); an associated signal, or a plurality of associated signals is typically detected at an analyte binding site to increase certainty in the presence and detectability of the single analyte. Accordingly, signals associated with a single analyte may be detected at some regularity or interval to determine presence and/or detectability of the single analyte at an analyte binding site. As the total number of cycles or steps in a single-analyte assay or process increases, it may be necessary to detect a signal associated with a single analyte at an analyte binding site within some temporal proximity to a final cycle or step of the assay or process. For example, in a 100 cycle assay, a failure to observe a signal at an array site during the final 50 cycles may lead to a conclusion of non-detectability of an analyte at the site, whereas detecting a signal at an array site during any cycle of the final 5 cycles of the assay may increase confidence that the analyte remained detectable throughout the 100 cycles.

An extent of photodamage protection provided to a single-analyte array may depend upon the length of a single-analyte assay or process (e.g., in terms of total cycles or total steps, in terms of total number of cycles or steps with irradiation). For example, photodamage protection provided for a 10 cycle assay may be less than that provided for a 100 cycle assay due to an increased likelihood of detrimental photodamage throughout the larger number of cycles. Accordingly, array, assay or process design may be varied with respect to quantity of photodamage inhibitor contacted or coupled to an array, and frequency of contacting or coupling of photodamage inhibitors to an array. A method, as set forth herein, may be designed to provide a minimum quantity of detectable analytes at the end of a number of cycles or steps of a single-analyte assay or process, for example as determined by presence of detectable signals from detectable probes at analyte binding sites. Single analytes may be detected from a threshold percentage of sites of a plurality of sites on a single-analyte array, such as at least 10%, 20%, 30%, 40%, 50%, 60%, 70%, 75%, 80%, 85%, 90%, 95%, 99%, 99.9%, or more than 99.9% of sites of the plurality of sites during or at the conclusion of a single-analyte assay or process. Single analytes may be detected from a threshold percentage of array sites of a plurality of sites within a temporal proximity to a conclusion of a single-analyte assay or process, such as within about 100, 75, 50, 40, 30, 20, 15, 10, 5, 4, 3, 2, or less than 2 cycles or steps of the conclusion of the single-analyte assay or process.

A single-analyte assay or process may comprise at least about 2, 3, 4, 5, 10, 15, 20, 25, 30, 40, 50, 60, 70, 75, 80, 90, 100, 125, 150, 175, 200, 250, 300, 400, 500, 750, 1000, 2000, 5000, 10000, or more than 10000 cycles or steps. Alternatively or additionally, a single-analyte assay or process may comprise no more than about 10000, 5000, 2000, 1000, 750, 500, 400, 300, 250, 200, 175, 150, 125, 100, 90, 80, 75, 70, 60, 50, 40, 30, 25, 20, 15, 10, 5, 4, 3, 2, or less than 2 cycles or steps.

A single-analyte assay or process may comprise a plurality of detection events, in which each detection event comprises at least one step of: i) binding detectable probes to single analytes on a single-analyte array, ii) after binding detectable probes to single analytes, providing an electromagnetic radiation field to the single-analyte array, and iii) detecting signals from analyte binding sites after providing the electromagnetic radiation field. In some cases, each cycle or sequence of steps of a single-analyte assay or process may comprise a detection event. A signal (e.g., an electromagnetic signal from a detectable probe) may be detected from a site of a plurality of sites during at least about 1, 2, 3, 4, 5, 10, 15, 20, 25, 30, 40, 50, 60, 70, 75, 80, 90, 100, 125, 150, 175, 200, 250, 300, 400, 500, 750, 1000, 2000, 5000, 10000 detection events. Alternatively or additionally, a signal may be detected from a site of a plurality of sites during no more than about 10000, 5000, 2000, 1000, 750, 500, 400, 300, 250, 200, 175, 150, 125, 100, 90, 80, 75, 70, 60, 50, 40, 30, 25, 20, 15, 10, 5, 4, 3, 2, or less than 2 detection events.

In an aspect, provided herein is a method, comprising: a) providing an array comprising a plurality of sites, in which each site of the plurality of sites comprises one and only one analyte of a plurality of analytes, and in which each site of the plurality of sites is resolvable at single-analyte resolution, b) coupling detectable probes to analytes of the plurality of analytes, in which each detectable probe comprises a fluorescent moiety that produces a detectable signal in the presence of an electromagnetic radiation field, in which each detectable probe comprises a nucleic acid nanostructure, and in which each detectable probe further comprises a photodamage inhibitor, c) at each site of the plurality of sites, detecting presence or absence of the detectable signal, in which detecting the presence or absence of the detectable signal comprises providing the electromagnetic radiation field, d) after detecting the presence or absence of the detectable signal, separating the detectable probes from the analytes, e) after separating the detectable probes from the analytes, detecting an absence of the detectable signal at each site of the plurality of sites.

In some cases, a method may further comprise coupling photodamage inhibitors to detectable probes. A method may comprise covalently coupling photodamage inhibitors to detectable probes. A method may comprise non-covalently coupling photodamage inhibitors to detectable probes. A method may comprise coupling photodamage inhibitors to detectable probes after coupling detectable probes to analytes. For example, FIGS. 4A-4C depict a method of coupling pendant moieties comprising photodamage inhibitors to a detectable probe. In other cases, a method may comprise coupling photodamage inhibitors to detectable probes before coupling detectable probes to analytes. In some cases, coupling photodamage inhibitors to detectable probes may comprise incorporating photodamage inhibitors within a structure of a detectable probe. For example, a photodamage inhibitor may be coupled to a nucleic acid intercalating compound, in which the intercalating compound couples the photodamage inhibitor to a nucleic acid retaining component of a detectable probe. In other cases, coupling photodamage inhibitors to detectable probes may comprise coupling a pendant moiety to a detectable probe. In particular cases, a method may comprise coupling a plurality of pendant moieties to a detectable probe, in which each pendant moiety comprises a plurality of photodamage inhibitors. A method may comprise coupling at least about 1, 2, 3, 4, 5, 6, 7, 8, 9, 10, 15, 20, 25, 30, 40, 50, 75, 100, 200, 500, 1000, or more than 1000 pendant moieties to a detectable probe. Alternatively or additionally, a method may comprise coupling no more than about 1000, 500, 200, 100, 75, 50, 40, 30, 25, 20, 15, 10, 9, 8, 7, 6, 5, 4, 3, 2, or less than 2 pendant moieties to a detectable probe.

In another aspect, provided herein is a method comprising: a) providing an array comprising a plurality of sites, in which each site of the plurality of sites comprises one and only one analyte of a plurality of analytes, in which each site further comprises an anchoring moiety, in which the anchoring moiety comprises a nucleic acid nanostructure, in which the anchoring moiety couples the one and only analyte to the array, in which the anchoring moiety further comprises a photodamage inhibitor, and in which each site of the plurality of sites is resolvable at single-analyte resolution, b) contacting the array with an electromagnetic radiation field at least two times, and c) after contacting the array with an electromagnetic radiation field at least two times, detecting at each site of the plurality of sites the presence of one and only one polypeptide of the plurality of sample polypeptides.

In some cases, a method may further comprise coupling photodamage inhibitors to anchoring moieties. A method may comprise covalently coupling photodamage inhibitors to anchoring moieties. A method may comprise non-covalently coupling photodamage inhibitors to anchoring moieties. A method may comprise coupling photodamage inhibitors to anchoring moieties after coupling detectable probes to analytes. In other cases, a method may comprise coupling photodamage inhibitors anchoring moieties before coupling detectable probes to analytes. In some cases, coupling photodamage inhibitors to anchoring moieties may comprise incorporating photodamage inhibitors within a structure of an anchoring moiety. For example, a photodamage inhibitor may be coupled to a nucleic acid intercalating compound, in which the intercalating compound couples the photodamage inhibitor to the nucleic acid nanostructure of an anchoring moiety. In other cases, coupling photodamage inhibitors to anchoring moieties may comprise coupling a pendant moiety to an anchoring moiety. In particular cases, a method may comprise coupling a plurality of pendant moieties to an anchoring moiety, in which each pendant moiety comprises a plurality of photodamage inhibitors. A method may comprise coupling at least about 1, 2, 3, 4, 5, 6, 7, 8, 9, 10, 15, 20, 25, 30, 40, 50, 75, 100, 200, 500, 1000, or more than 1000 pendant moieties to an anchoring moiety. Alternatively or additionally, a method may comprise coupling no more than about 1000, 500, 200, 100, 75, 50, 40, 30, 25, 20, 15, 10, 9, 8, 7, 6, 5, 4, 3, 2, or less than 2 pendant moieties to an anchoring moiety.

It may be advantageous to control a location and/or orientation of photodamage inhibitors on a single-analyte array. Coupling of photodamage inhibitors in close proximity to analytes or other degradable species (e.g., analytes) may facilitate reduced energy transfer to the analytes or other degradable species, thereby decreasing photodamage. Without wishing to be bound by theory, two entities separated by a distance of substantially less than the wavelength of a photon of light may compete to absorb the photon. Coupling a photon-scavenging photodamage inhibitor close to an analyte (e.g., at a distance less than a wavelength of impinging photons) may reduce the likelihood of a photon being absorbed by the analyte. Coupling a photon-scavenging photodamage inhibitor close to a detectable label may reduce the likelihood of a photon being transmitted to a detection system (e.g., an optical sensor). Accordingly, photodamage inhibitors, as set forth herein, may be selectively located on a single-analyte array to facilitate dissipation of excess photon energy in one or more first regions of a single-analyte array (e.g., at interstitial regions, within a fluidic medium), and to facilitate detectable signal transmission at one or more second regions of a single-analyte array (e.g., at analyte binding sites).

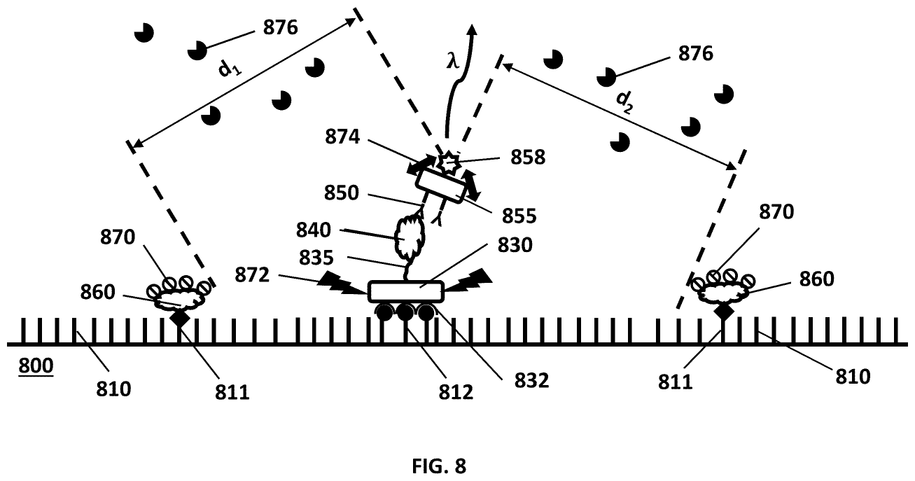

FIG. 8 illustrates a configuration of a single-analyte array comprising multiple types of photodamage inhibitors with differing spatial separations from other array entities. A single-analyte array may comprise a solid support 800 comprising interstitial regions containing surface-coupled passivating moieties 810, and analyte binding sites containing surface-coupled coupling moieties 812. Interstitial regions and/or analyte binding sites may further comprise defects 811 that coupled macromolecular structures 860 of a blocking reagent. Analytes 840 are attached to anchoring moieties 830 by a linking moiety 835, and anchoring moieties 830 are coupled to analyte binding sites by coupling of surface-coupled coupling moieties 812 to complementary coupling moieties 832 of the anchoring moieties 830. A detectable probe comprising a retaining component 855, a plurality of affinity agents 850, and a detectable label 858, is bound to an analyte 840, thereby co-locating the detectable probe and the analyte at a single analyte binding site. The detectable label 858 emits a photon of light of wavelength λ, thereby transmitting a detectable signal. Multiple types of photodamage inhibitors are coupled to array entities or components. Macromolecular structures 860 comprise pluralities of quenching moieties, 870. Anchoring moieties 830 comprise photolabile moieties 872. Retaining components 855 of detectable probes comprise photoisomerization moieties 874. A fluidic medium contacted with the single-analyte array comprise reactive scavengers 876. The quenching moieties 870 are located at minimum distances d₁ and d₂, in which d₁ and d₂ are greater than wavelength λ, thereby reducing the likelihood of quenching photons emitted by detectable labels 858. The photolabile moieties 872 and photoisomerization moieties 874 may be located at distances less than wavelength λ if the absorption range of these moieties does not significantly overlap with wavelength λ. Accordingly, in a system with multiplexed detection (e.g., using multiple wavelengths of light), photodamage inhibitors may be selected and located to provide orthogonal photodamage protection for a particular wavelength of light. For example, in a multiplexed detection system utilizing light of wavelength λ₁ and wavelength λ₂, detectable probes comprising detectable labels that emit at wavelength λ₁ may comprise photon-scavenging photodamage inhibitors that absorb light of wavelength λ₂. The skilled person will readily recognize innumerable alternative variations of type and location of photodamage inhibitors relative to FIG. 8.

A photodamage inhibitor moiety may be spatially separated from an array entity (e.g., a surface-coupled moiety, an anchoring moiety, an analyte, an affinity agent, a retaining component, a detectable label, etc.) by an average distance of at least about 0.1 nanometers (nm), 0.5 nm, 1 nm, 2 nm, 3 nm, 4 nm, 5 nm, 10 nm, 15 nm, 20 nm, 25 nm, 30 nm, 40 nm, 50 nm, 75 nm, 100 nm, 150 nm, 200 nm, 300 nm, 400 nm, 500 nm, or more than 500 nm. Alternatively or additionally, a photodamage inhibitor moiety may be spatially separated from an array entity by an average distance of no more than about 500 nm, 400 nm, 300 nm, 200 nm, 150 nm, 100 nm, 75 nm, 50 nm, 40 nm, 30 nm, 25 nm, 20 nm, 15 nm, 10 nm, 5 nm, 4 nm, 3 nm, 2 nm, 1 nm, 0.5 nm, 0.1 nm, or less than 0.1 nm. An average distance between a photodamage inhibitor moiety and an array entity may be determined due to natural thermal fluctuation in separation, as well as fluctuations in separation distance that can arise due to rotational, translational, or vibrational variations of positions, for example natural motion of a pendant moiety comprising a photodamage inhibitor.

It may be preferable to provide a plurality of photodamage inhibitor moieties, as set forth herein, for each array site or each entity bound to an array site (e.g., an analyte, an anchoring moiety, a binding reagent, a combination thereof, etc.). A single-analyte array system, including an array, entities bound to the array, and optionally a fluidic medium contacted to the array, may comprise an average of at least about 1, 10, 50, 100, 200, 300, 400, 500, 600, 700, 800, 900, 1000, 2000, 3000, 4000, 5000, 10000, 50000, 100000, 1000000, or more than 1000000 photodamage inhibitor moieties per array site or each entity bound thereto. Alternatively or additionally, a single-analyte array system, including an array, entities bound to the array, and optionally a fluidic medium contacted to the array, may comprise an average of no more than about 1000000, 100000, 50000, 10000, 5000, 4000, 3000, 2000, 1000, 900, 800, 700, 600, 500, 400, 300, 200, 100, 50, 10, or less than 10 photodamage inhibitor moieties per array site or each entity bound thereto.

The present disclosure provides single-analyte array systems comprising photodamage inhibitor moieties, including photodamage inhibitor moieties that are bound to arrays, or bound entities attached to arrays. A single-analyte array system can further comprise a fluidic medium comprising photodamage inhibitors that is contacted to an array. A fluidic medium can comprise a plurality of photodamage inhibitors that are solubilized in the fluidic medium, including antioxidants, reactive oxygen scavengers, radical scavengers, photolabile compounds, and photoisomerization compounds. In some cases, a fluidic medium may comprise two or more types of species of photodamage inhibitors (e.g., an antioxidant species and a reactive oxygen scavenger species). Exemplary photodamage inhibitors provided to a fluidic medium can include ascorbic acid, 9,10-anthracenediyl-bis(methylene) dimalonic acid (ABDA), epigallocatechin gallate (EPGG), N-acetyl-L-cysteine, caffeic acid, reseveratrol, 4-hydroxy-2,2,6,6-tetramethylpiperidin-1-oxyl (TEMPOL), sodium sulfite, 1,4-diazabicyclo [2.2.2]octane (DABCO), sodium pyruvate, N,N′-dimethylthiourea (DMTU), mannitol, dimethyl sulfoxide (DMSO), 6-hydroxy-2,5,7,8-tetramethylchroman-2-carboxylic acid (Trolox), 2-phenyl-1,2-benzisoselenazol-3(2H)-one (Ebselen), α-tocopherol, uric acid, sodium azide, manganese(III)-tetrakis(4-benzoic acid) porphyrin, 4,5-dihydroxybenzene-1,3-disulfonate, and combinations thereof.

Accordingly, a plurality of photodamage inhibitors may be provided to a single-analyte array system, in which a first fraction of the photodamage inhibitors (on a molar or mass basis) are attached to the array, and in which a second fraction of the photodamage inhibitors (on a molar or mass basis are provided in a fluidic medium contacted to the array. The first fraction or the second fraction of photodamage inhibitors may have a fraction (on a mass or molar basis) of at least about 0.01, 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 0.99, or more than 0.99. Alternatively or additionally, the first fraction or second fraction of photodamage inhibitors may have a fraction (on a mass or molar basis) of no more than about 0.99, 0.95, 0.9, 0.85, 0.8, 0.75, 0.7, 0.65, 0.6, 0.55, 0.5, 0.45, 0.4, 0.35, 0.3, 0.25, 0.2, 0.15, 0.1, 0.05, 0.01, or less than 0.01.

It may be preferable to provide a first fraction of photodamage inhibitor moieties (i.e., photodamage inhibitor moieties coupled to an array) and a second fraction of photodamage inhibitor moieties (i.e., photodamage inhibitor moieties in a fluidic medium) for each array site or each entity bound to an array site (e.g., an analyte, an anchoring moiety, a binding reagent, a combination thereof, etc.). A first fraction or a second fraction of photodamage inhibitor moieties may comprise an average of at least about 1, 10, 50, 100, 200, 300, 400, 500, 600, 700, 800, 900, 1000, 2000, 3000, 4000, 5000, 10000, 50000, 100000, 1000000, or more than 1000000 photodamage inhibitor moieties per array site or each entity bound thereto. Alternatively or additionally, a first fraction or a second fraction of photodamage inhibitor moieties may comprise an average of no more than about 1000000, 100000, 50000, 10000, 5000, 4000, 3000, 2000, 1000, 900, 800, 700, 600, 500, 400, 300, 200, 100, 50, 10, or less than 10 photodamage inhibitor moieties per array site or each entity bound thereto.

A method provided by the present disclosure may comprise one or more steps of: i) contacting a plurality of probes, binding reagents, or molecules to a single-analyte array, as set forth herein, ii) binding or associating probes, binding reagents, or molecules of the plurality of probes, binding reagents, or molecules to entities bound to array sites of the single-analyte array, iii) detecting a presence or absence of a probe, binding reagent, or molecule at each individual site of a plurality of sites of the single-analyte array, optionally in the presence of photons (e.g., fluorescence- or luminescence-based detection) and iv) dissociating probes, binding reagents or molecules from the single-analyte array. The methods of utilizing photodamage inhibitor moieties set forth herein may be advantageous for facilitating the dissociation of probes, binding reagents, or molecules from array-bound entities. In the absence of the photodamage inhibitor moieties, unwanted or unexpected deleterious photon-mediated interactions may occur, such as cross-linking a probe, binding reagent, or molecule at an array site, thereby inhibiting dissociation of the probe, binding reagent, or molecule.

In some cases, probes, binding reagents, or molecules may bind or associate with at least about 0.01%, 0.1%, 0.5%, 1%, 5%, 10%, 15%, 20%, 25%, 30%, 35%, 40%, 50%, 60%, 70%, 80%, 90%, 95%, 99%, 99.9%, 99.99%, or more than 99.99% of sites of a single-analyte array or with sites of a single-analyte array comprising an entity (e.g., a macromolecule, an analyte, etc.). Alternatively or additionally, probes, binding reagents, or molecules may bind or associate with no more than about 99.99%, 99.9%, 99%, 95%, 90%, 80%, 70%, 60%, 50%, 40%, 35%, 30%, 25%, 20%, 15%, 10%, 5%, 1%, 0.5%, 0.1%, 0.01%, or less than 0.01% of sites of a single-analyte array or with sites of a single-analyte array comprising an entity. A percentage of sites that bind a probe, binding reagent, or molecule may depend upon the nature of the probes, binding reagents, or molecules, and the nature of the array-bound entities. For example, affinity agents (e.g., antibodies, aptamers, etc.) may bind to a lower percentage of sites containing two or more unique species of polypeptides than an Edman-type degradation reactant that would be expected to interact with substantially all polypeptides of a polypeptide array.

In some cases, probes, binding reagents, or molecules may be detected at at least about 0.01%, 0.1%, 0.5%, 1%, 5%, 10%, 15%, 20%, 25%, 30%, 35%, 40%, 50%, 60%, 70%, 80%, 90%, 95%, 99%, 99.9%, 99.99%, or more than 99.99% of sites of a single-analyte array or at sites of a single-analyte array comprising an entity (e.g., a macromolecule, an analyte, etc.). Alternatively or additionally, probes, binding reagents, or molecules may be detected at no more than about 99.99%, 99.9%, 99%, 95%, 90%, 80%, 70%, 60%, 50%, 40%, 35%, 30%, 25%, 20%, 15%, 10%, 5%, 1%, 0.5%, 0.1%, 0.01%, or less than 0.01% of sites of a single-analyte array or at sites of a single-analyte array comprising an entity.

In some cases, probes, binding reagents, or molecules may be dissociated from at least about 0.01%, 0.1%, 0.5%, 1%, 5%, 10%, 15%, 20%, 25%, 30%, 35%, 40%, 50%, 60%, 70%, 80%, 90%, 95%, 99%, 99.9%, 99.99%, or more than 99.99% of sites of a single-analyte array or from sites of a single-analyte array comprising an entity (e.g., a macromolecule, an analyte, etc.). Alternatively or additionally, probes, binding reagents, or molecules may be dissociated from no more than about 99.99%, 99.9%, 99%, 95%, 90%, 80%, 70%, 60%, 50%, 40%, 35%, 30%, 25%, 20%, 15%, 10%, 5%, 1%, 0.5%, 0.1%, 0.01%, or less than 0.01% of sites of a single-analyte array or from sites of a single-analyte array comprising an entity. In some cases, probes, binding reagents, or molecules may be dissociated from at least about 0.01%, 0.1%, 0.5%, 1%, 5%, 10%, 15%, 20%, 25%, 30%, 35%, 40%, 50%, 60%, 70%, 80%, 90%, 95%, 99%, 99.9%, 99.99%, or more than 99.99% of sites of a single-analyte array where a signal from a probe, binding reagent, or molecule was detected. Alternatively or additionally, probes, binding reagents, or molecules may be dissociated from no more than about 99.99%, 99.9%, 99%, 95%, 90%, 80%, 70%, 60%, 50%, 40%, 35%, 30%, 25%, 20%, 15%, 10%, 5%, 1%, 0.5%, 0.1%, 0.01%, or less than 0.01% of sites of a single-analyte array where a signal from a probe, binding reagent, or molecule was detected.

### Compositions Comprising Photodamage Inhibitors

Described herein are single-analyte array compositions comprising coupled photodamage inhibitors. The compositions may be useful for providing increased protection of analytes during single-analyte assays or processes that involve repeated or long-duration exposure of the analytes to electromagnetic radiation. Particularly advantageous compositions may comprise photodamage inhibitors coupled to a single-analyte array via one or more nucleic acid nanostructures. Nucleic acid nanostructures may provide a convenient and tunable platform for coupling together various combinations of array components, such as analytes, detectable labels, affinity agents, and photodamage inhibitors.

In an aspect, provided herein is an array composition, comprising: a) a plurality of sites, in which each individual site of the plurality of sites comprises one and only one macromolecules of a first plurality of macromolecules, b) at each individual site of a fraction of sites of the plurality of sites, a macromolecule of a second plurality of macromolecules bound to the one and only one macromolecule of the first plurality of macromolecules, and c) at each individual site of the fraction of sites of the plurality of sites, a plurality of photodamage inhibitor moieties coupled to each individual site, in which the fraction of sites contains an average of at least 100 photodamage inhibitor moieties per site.

In an aspect, provided herein is a composition comprising: a) a nucleic acid nanostructure, in which the nucleic acid nanostructure comprises a first face and a second face, in which the first face and second face comprise differing average orientations, b) a biomolecule covalently coupled to the first face of the nucleic acid nanostructure, and c) a plurality of photodamage inhibitors coupled to the second face of the nucleic acid nanostructure. Optionally, the biomolecule is constrained from contacting the photodamage inhibitors due to the conformation of the nucleic acid nanostructure and the covalent attachment of the biomolecule and photodamage inhibitors to the respective faces of the nucleic acid nanostructures. The orientation of the faces can further constrain the biomolecule and photodamage inhibitors such that the photodamage inhibitors are inhibited from quenching fluorophores that are attached to the biomolecule, such as fluorophores present on an affinity agent that is bound to the biomolecule. In other cases, a nucleic acid nanostructure may comprise a face, in which a biomolecule and a plurality of photodamage inhibitors are coupled to the face. Optionally, the biomolecule is constrained to maintain a distance from the photodamage inhibitors such that the biomolecule does not contact the photodamage inhibitors. The distance can also inhibit quenching of fluorophores attached to the biomolecule by the photodamage inhibitors.

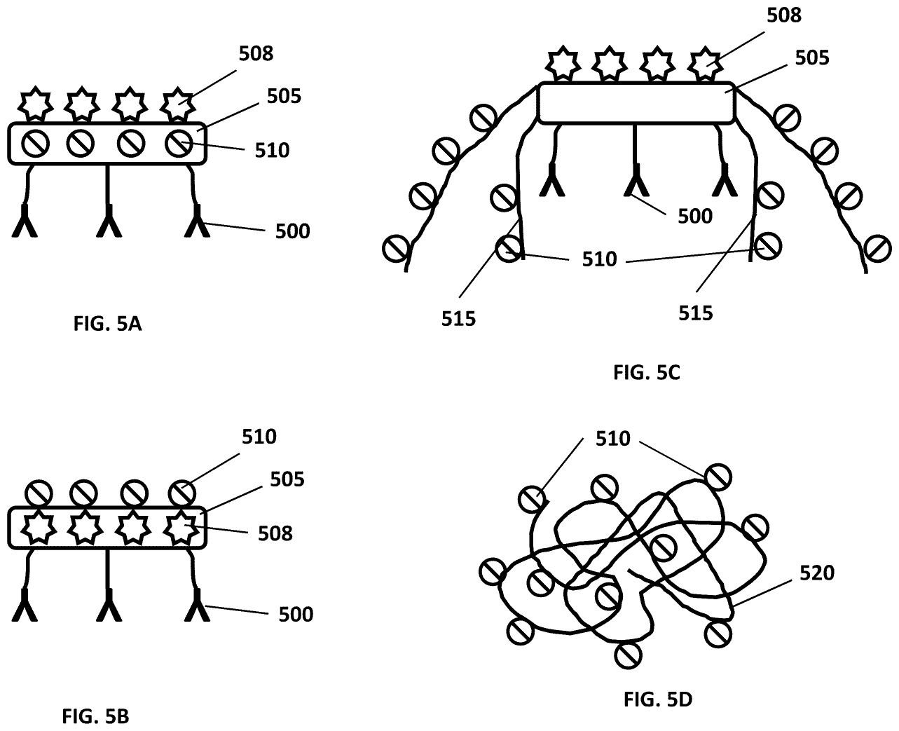

FIGS. 5A-5D depict. useful compositions for single-analyte assays and processes set forth herein. FIGS. 5A-5C depict configurations of detectable probes that may be utilized to bind to and/or characterize single analytes on a single-analyte array. FIG. 5A depicts a detectable probe comprising a plurality of affinity agents 500 (e.g., antibodies, antibody fragments, aptamers, etc.), in which each affinity agent 500 is coupled to a retaining component 505 (e.g., a nucleic acid nanostructure, a polymeric nanoparticle, an inorganic nanoparticle, a carbon nanoparticle, etc.). The detectable probe further comprises a plurality of detectable labels 508 that are coupled to the detectable probe on a face of the retaining component 505 that is substantially opposite a face to which the plurality of affinity agents 500 is coupled, and a plurality of photodamage inhibitors 510 that are coupled within a structure of the retaining component 505 or on a differing face from the affinity agents 500 and detectable labels 508. FIG. 5B depicts a similar detectable probe configuration as FIG. 5A, however detectable labels 508 and photodamage inhibitors 510 are coupled to differing faces or regions of the retaining component 505 structure. A skilled person will readily recognize innumerable variations of location and orientation for detectable probe components, including coupling detectable labels 508 and photodamage inhibitors 510 on a same face of a retaining component 505, coupling affinity agents 500 and photodamage inhibitors 510 on a same face of the retaining component 505, and combinations thereof. A nucleic acid nanostructure may be a particularly useful retaining component 505 due to its tunable nature with regard to location and orientation for coupling components (e.g., affinity agents 500, detectable labels 508, photodamage inhibitors 510) to the nanostructure. FIG. 5C depicts a detectable probe configuration in which the affinity agents 500 and detectable labels 508 are coupled to the retaining component 505 similarly to the composition of FIG. 5A. The detectable probe comprises a plurality of pendant moieties 515 (e.g., oligonucleotides, polymeric chains) that are coupled to the retaining component 505. Each pendant moiety 515 comprises a plurality of coupled photodamage inhibitors 510. FIG. 5D depicts a macromolecular structure 520 (e.g., a polymeric chain, a polysaccharide, a polypeptide) that comprises a plurality of coupled photodamage inhibitors 510. A macromolecular structure may be included as a component of a blocking agent, in which the blocking agent is configured to passivate orthogonal binding sites on an array surface.

A nucleic acid nanoparticle may comprise two or more oligonucleotides that form a structure of the nucleic acid nanoparticle through base-pair hybridization interactions. Two-dimensional and/or three-dimensional structures associated with nucleic acid nanostructures may arise, in part, due to self-complementarity hybridization interactions of a single oligonucleotide within the nucleic acid nanostructure. Additionally or alternatively, two-dimensional and/or three-dimensional structures associated with nucleic acid nanostructures may arise, in part, due to hybridization interactions between two or more oligonucleotides of a nucleic acid nanoparticle. In some cases, two-dimensional and/or three-dimensional structures associated with nucleic acid nanostructures may arise, in part, due to hybridization interactions between a first oligonucleotide and two or more non-contiguous nucleotide sequences of a second oligonucleotide. For example, a nucleic acid origami may be formed by repeated folding of a scaffold oligonucleotide due to hybridization of staple oligonucleotides, in which staple oligonucleotides bind to at least two non-contiguous sequences of the scaffold oligonucleotide. In some cases, a two-dimensional and/or three-dimensional structures associated with nucleic acid nanostructures may arise, in part, due to a hybridization interaction(s) between a first oligonucleotide and part of a second oligonucleotide. For example, pendant single-stranded oligonucleotide may be formed by partial hybridization of a first oligonucleotide to a second oligonucleotide, in which a terminal nucleotide sequence or an intermediate nucleotide sequence of the first oligonucleotide is of sufficient length to form a pendant single stranded nucleic acid. In some cases, a nucleic acid nanoparticle may comprise a single oligonucleotide, in which a structure of the nucleic acid nanoparticle arises due to internal self-complementarity of nucleotide sequences for complementary nucleic acid sequences of the single oligonucleotide (e.g., a nucleic acid nanoball comprising a concatemer of a self-complementary nucleotide sequence).

A nucleic acid nanoparticle may comprise at least two oligonucleotides. A nucleic acid nanoparticle may comprise a plurality of oligonucleotides, in which each oligonucleotide is at least partially hybridized to at least one other oligonucleotide of the plurality of oligonucleotides. A nucleic acid nanoparticle may comprise at least about 2, 3, 4, 5, 10, 20, 25, 30, 40, 50, 75, 100, 125, 150, 175, 200, 250, 500, or more than 500 oligonucleotides. Alternatively or additionally, a nucleic acid nanoparticle may comprise no more than about 500, 250, 200, 175, 150, 125, 100, 75, 50, 40, 30, 25, 20, 10, 5, 4, 3, 2, or less than 2 oligonucleotides.

In some cases, a nucleic acid nanoparticle may comprise a scaffold oligonucleotide. A scaffold oligonucleotide may be hybridized to a plurality of staple oligonucleotides to form a particular two-dimensional or three-dimensional structure of a nucleic acid nanoparticle. A scaffold oligonucleotide may be modified, for example by the inclusion of non-natural or modified nucleotides, thereby permitting attachment of entities (e.g., a single analyte, a solid support, a detectable label, a photodamage inhibitor, a surface-coupled moiety) to the scaffold oligonucleotide. A scaffold oligonucleotide may be modified to alter a conformation of a nucleic acid nanoparticle.

A nucleic acid nanoparticle may comprise a plurality of staple oligonucleotides. A staple oligonucleotide may comprise any oligonucleotide that is hybridized with, or configured to hybridize with, a nucleic acid scaffold, other staples, or a combination thereof. A staple oligonucleotide may be modified to include additional chemical entities, such as binding components, label components, chemically-reactive groups or handles, or other groups (e.g., polyethylene glycol (PEG) moieties). A staple oligonucleotide may comprise linear or circular nucleic acids. A staple oligonucleotide may comprise one or more single-stranded regions, double-stranded regions, or combinations thereof. A staple oligonucleotide may be hybridized with, or configured to hybridize with, a scaffold strand or one or more other staples, for example, via complementary base pair hybridization (e.g., Watson-Crick hybridization). A staple oligonucleotide may be hybridized with other nucleic acids by complementary base pair hybridization or ligation. A staple oligonucleotide may be configured to act as a primer for a complementary nucleic acid strand and the primer staple may be extended by an enzyme (e.g., a polymerase) to form lengthened regions of double-stranded nucleic acid, for example, using a scaffold, staple or other strand as a template. In some cases the primer need not be hybridized to a template when extended. For example, a primer can be extended by template-free addition of one or more nucleotides by a terminal transferase enzyme, by template-free addition of one or more oligonucleotides by a ligase enzyme or template-free addition of nucleotide(s) or oligonucleotide(s) by non-enzymatic chemical reaction. A staple oligonucleotide may include one or more modified nucleotides. A modified nucleotide may include a linking group or a reactive handle (e.g., a functional group configured to perform a click-type reaction). A modified staple oligonucleotide may facilitate attachment of entities (e.g., a single analyte, a solid support, a surface-coupled moiety) to the staple oligonucleotide.

A staple oligonucleotide may be any length depending upon the design of the SNAP. A staple oligonucleotide may be designed by a software package, such as caDNAno2, ATHENA, OR DAEDALUS. A staple oligonucleotide may have a length of at least about 10, 25, 50, 100, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, 2000, 2500, 3000, 3500, 4000, 4500, 5000, or more than 5000 nucleotides. Alternatively or additionally, a staple may have a length of no more than about 5000, 4500, 4000, 3500, 3000, 2500, 2000, 1900, 1800, 1700, 1600, 1500, 1400, 1300, 1200, 1100, 1000, 950, 900, 850, 800, 750, 700, 650, 600, 550, 500, 450, 400, 350, 300, 250, 200, 150, 100, 50, 25, 10, or less than 10 nucleotides.

A nucleic acid nanoparticle may be formed by hybridization of two or more oligonucleotides. A stability of any hybridization interaction within a nucleic acid nanoparticle may depend at least in part on factors such as a total number of base-paired nucleotides, presence of non-paired nucleotides within a base-paired nucleotide sequence, and GC content of a base-paired nucleotide sequence. Nucleic acid melting temperature may be a useful proxy for relative stability of a nucleic acid hybridization interaction. Typically, a higher nucleic acid melting temperature suggests a more stable binding interaction. A binding interaction within a nucleic acid nanoparticle may be designed facilitate or inhibit dissociation of the binding interaction. For example, a detectable probe may comprise a nucleic acid nanoparticle, in which a detectable label is coupled to the nucleic acid nanoparticle by oligonucleotide hybridization, and in which the oligonucleotide comprising the detectable label is configured to have a lower melting temperature than an average melting temperature of the nucleic acid nanoparticle. In another example, an anchoring moiety may comprise a plurality of pendant single-stranded nucleic acids, in which the pendant single-stranded nucleic acids attach to surface-coupled oligonucleotides, and in which an average melting temperature of hybridization interactions of pendant single-stranded nucleic acids with surface-coupled oligonucleotides is at least as high as an average melting temperature of the nucleic acid nanoparticle.

A nucleic acid nanoparticle may comprise a first oligonucleotide attached to a second oligonucleotide by a hybridization interaction, in which the hybridization interaction has a characterized melting temperature. A hybridization interaction between a first oligonucleotide and a second oligonucleotide may have a melting temperature of at least about 48° C., 49° C., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C., 75° C., 76° C., 77° C., 78° C., 79° C., 80° C., 81° C., 82° C., 83° C., 84° C., 85° C., 86° C., 87° C., 88° C., 89° C., 90° C., or more than 90° C. Alternatively or additionally, a hybridization interaction between a first oligonucleotide and a second oligonucleotide may have a melting temperature of no more than about 90° C., 89° C., 88° C., 87° C., 86° C., 85° C., 84° C., 83° C., 82° C., 81° C., 80° C., 79° C., 78° C., 77° C., 76° C., 75° C., 74° C., 73° C., 72° C., 71° C., 70° C., 69° C., 68° C., 67° C., 66° C., 65° C., 64° C., 63° C., 62° C., 61° C., 60° C., 59° C., 58° C., 57° C., 56° C., 55° C., 54° C., 53° C., 52° C., 51° C., 50° C., 49° C., 48° C., or less than 48° C.

A nucleic acid nanoparticle may comprise a plurality of nucleic acid hybridization interactions, in which the plurality of nucleic acid hybridization interactions comprises an average characterized melting temperature. A plurality of hybridization interactions may have an average melting temperature of at least 48° C., 49° C., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C., 75° C., 76° C., 77° C., 78° C., 79° C., 80° C., 81° C., 82° C., 83° C., 84° C., 85° C., 86° C., 87° C., 88° C., 89° C., 90° C., or more than 90° C. Alternatively or additionally, a plurality of hybridization interaction may have an average melting temperature of no more than about 90° C., 89° C., 88° C., 87° C., 86° C., 85° C., 84° C., 83° C., 82° C., 81° C., 80° C., 79° C., 78° C., 77° C., 76° C., 75° C., 74° C., 73° C., 72° C., 71° C., 70° C., 69° C., 68° C., 67° C., 66° C., 65° C., 64° C., 63° C., 62° C., 61° C., 60° C., 59° C., 58° C., 57° C., 56° C., 55° C., 54° C., 53° C., 52° C., 51° C., 50° C., 49° C., 48° C., or less than 48° C.

A method set forth herein may involve formation of interactions that are intended to remain associated throughout a single-analyte assay or process. Alternatively or additionally, a method set forth herein may involve formation of interactions that are intended to become dissociated during a single-analyte assay or process. For example, non-irradiated photodamage inhibitors may be coupled to nucleic acid nanoparticles repeatedly during an assay, while irradiated photodamage inhibitors may be removed from the nucleic acid nanoparticles. During such an assay, the nucleic acid nanoparticles may remain associated to a solid support throughout the assay. Accordingly, it may be advantageous to alter stability of particular hybridization interactions that form an anchoring moiety or a constituent thereof (e.g., a nucleic acid nanoparticle). For example, an oligonucleotide comprising photodamage inhibitors may be coupled to an oligonucleotide of a nucleic acid nanoparticle by a hybridization interaction, in which the hybridization interaction must be dissociable as the photodamage inhibitors become depleted. In another example, a composition such as the one depicted in FIG. 4C may be formed, in which the pendant moieties comprising photodamage inhibitors, and preferably the detectable probe, must be dissociated to permit subsequent binding of a new detectable probe.

Accordingly, a particular nucleic acid hybridization interaction may be designed to have increased or decreased stability with respect to another nucleic acid hybridization interaction or another network of nucleic acid hybridization interactions. In some cases, a first nucleic acid hybridization interaction within a detectable probe or a constituent thereof may be more stable or less stable than a second nucleic acid hybridization interaction within a detectable probe or a constituent thereof. In some cases, a nucleic acid hybridization interaction within a detectable probe or a constituent thereof may be more stable or less stable than a nucleic acid hybridization interaction within an anchoring moiety or a constituent thereof. In some cases, a first nucleic acid hybridization interaction within an anchoring moiety or a constituent thereof may be more stable or less stable than a second nucleic acid hybridization interaction within an anchoring moiety or a constituent thereof. In some cases, a nucleic acid hybridization interaction within an anchoring moiety, a detectable probe, or a constituent thereof may be more stable or less stable than a nucleic acid hybridization interaction within an anchoring moiety, a detectable probe, or a constituent thereof. In some cases, a first nucleic acid hybridization interaction and a second nucleic acid hybridization interaction or network thereof may be designed to have a similar stability, for example as characterized by nucleic acid melting temperature.

A difference in stability between a particular nucleic acid hybridization interaction and another nucleic acid hybridization interaction or network thereof may be characterized by a differential in melting temperatures. In some cases, a differential in melting temperatures may be calculated as a difference in melting temperatures between a first hybridization interaction and a second hybridization interaction. In other cases, a differential in melting temperatures may be calculated as a difference in melting temperatures between a first hybridization interaction and an average of a plurality of hybridization interactions. A differential in melting temperatures may have an absolute value of at least about 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., or more than 30° C. Alternatively or additionally, a differential in melting temperatures may have an absolute value of no more than about 30° C., 29° C., 28° C., 27° C., 26° C., 25° C., 24° C., 23° C., 22° C., 21° C., 20° C., 129° C., 18° C., 17° C., 16° C., 15° C., 14° C., 13° C., 12° C., 11° C., 10° C., 9° C., 8° C., 7° C., 6° C., 5° C., 4° C., 3° C., 2° C., 1° C., or less than 1° C.

A nucleic acid nanoparticle, may have a particular number of faces. A nucleic acid nanoparticle may have at least about 1, 2, 3, 4, 5, 6, 7, 8, 9, 10, 11, 12, 13, 14, 15, 16, 17, 18, 19, 20, or more than 20 faces. Additionally or alternatively, a nucleic acid nanoparticle may have no more than about 20, 19, 18, 17, 16, 15, 14, 13, 12, 11, 10, 9, 8, 7, 6, 5, 4, 3, 2 or less than 2 faces. The number of faces of a nucleic acid nanoparticle may be chosen to match a functionality for the nucleic acid nanoparticle. For example, a nucleic acid nanoparticle that is configured to couple an analyte to a solid support may necessitate at least 2 faces (a display face and a coupling face), with additional faces added based upon other design considerations (e.g., utility faces). An orientation of a first face may be determined with respect to an orientation of a second face based upon an angular offset between a first vector that is normal to a plane defining an average spatial location of the first face and a second vector that is normal to a plane defining an average spatial location of the second face. In other configurations, an orientation of a first face may be offset from an orientation of a second face by at least about 90°. In other configurations, an orientation of a first face may be offset from an orientation of a second face by about 180°. A nucleic acid nanoparticle may comprise a first face and a second face with an angular offset of at least about 10°, 20°, 30°, 40°, 50°, 60°, 70°, 80°, 90°, 100°, 110°, 120°, 130°, 140°, 150°, 160°, 170°, 180°, 190°, 200°, 210°, 220°, 230°, 240°, 250°, 260°, 270°, 280°, 290°, 300°, 310°, 320°, 330°, 340°, 350°, or more than 350°. Alternatively or additionally, a nucleic acid nanoparticle may comprise a first face and a second face with an angular offset of no more than about 360°, 350°, 340°, 330°, 320°, 310°, 300°, 290°, 280°, 270°, 260°, 250°, 240°, 230°, 220°, 210°, 200°, 190°, 180°, 170°, 160°, 150°, 140°, 130°, 120°, 110°, 100°, 90°, 80°, 70°, 60°, 50°, 40°, 30°, 20°, 10°, or less than 10°. In some cases, an angular offset between a first face and a second face may substantially occlude contact with the second face of an entity coupled to the first face. In some cases, a face of a nucleic acid nanoparticle may have a characteristic dimension with a length scale that is optically resolvable, such as at least about 1 nanometer (nm), 2 nm, 3 nm, 4 nm, 5 nm, 10 nm, 15 nm, 20 nm, 25 nm, 30 nm, 40 nm, 50 nm, 100 nm, or more than 100 nm.

Stability of nucleic acid nanoparticles may be influenced by the presence of covalent cross-linking. In some cases, covalent cross-linking may occur between a first oligonucleotide of a nucleic acid nanoparticle and a second oligonucleotide of a nucleic acid nanoparticle (i.e., inter-strand cross-linking). In some cases, covalent cross-linking may occur within a single oligonucleotide of a nucleic acid nanoparticle (i.e., intra-strand cross-linking). Covalent cross-linking may occur due to contact of a nucleic acid cross-linking reagent, as set forth herein, with a nucleic acid or a nucleic acid nanoparticle. In some cases, a nucleic acid nanoparticle may comprise at least as many covalent cross-links as a quantity of oligonucleotides within the nucleic acid nanoparticle. In other cases, a nucleic acid nanoparticle may comprise fewer covalent cross-links than a quantity of oligonucleotides within the nucleic acid nanoparticle. Covalent cross-linking may be particularly advantageous for nucleic acid nanoparticles of anchoring moieties due to a need to maintain stability of the nucleic acid nanoparticles, thereby preventing dissociation of a binding interaction, for example dissociation from analyte binding sites or dissociation of photodamage inhibitors.

Stability of nucleic acid nanoparticles may be influenced by the presence of cleavable linkers (e.g., photocleavable linkers, chemically-cleavable linkers, etc.). In some cases, cleavable linkers may be incorporated into a nucleic acid nanoparticles to facilitate decomposition of the nucleic acid nanoparticle. In some cases, a cleavable linker may be incorporated into a nucleic acid nanoparticles to facilitate dissociation of a particular component or moiety from the nucleic acid nanoparticle (e.g., a detectable label, an affinity agent). A cleavable linker may be dissociated by contacting a nucleic acid nanoparticle with a cleaving condition, such as light irradiation (for photocleavable linkers), contacting with a chemical cleaving agent (for chemically-cleavable linkers), or enzymatic digestion (e.g., restriction enzyme digestion). Incorporation of cleavable linkers may be particularly advantageous for detectable probes, whereby dissociation of detectable probes from analytes can be accomplished, at least in part, by decomposition of the detectable probe or dissociation of components from the probes.

A nucleic acid nanoparticle may comprise one or more pendant moieties, such as single-stranded nucleic acids, polymeric chains (e.g., PEG, alkane chains, etc.), components of a receptor-ligand binding pair (e.g., streptavidin-biotin, SpyCatcher-SpyTag, SnoopCatcher-SnoopTag, etc.), covalent reactive groups (e.g., NHS esters, Click-type reagents, etc.), or combinations thereof. A pendant moiety may be configured to couple a nucleic acid nanoparticle to a solid support. In some cases, a nucleic acid nanoparticle may be coupled to a solid support by binding interactions of a plurality of pendant moieties. In particular cases, pendant moieties may form non-covalent binding interactions, covalent binding interactions, or combinations thereof with a solid support or moieties attached thereto. It may be particularly advantageous to couple an anchoring moiety to an analyte binding site by at least one covalent binding interaction, thereby inhibiting dissociation of the anchoring moiety and/or analyte from the analyte binding site. A pendant moiety may be configured to couple a detectable label to a nucleic acid nanoparticle. For example, a nucleic acid nanoparticle of a detectable probe or an anchoring moiety may comprise a pendant single-stranded nucleic acid that forms a hybridization interaction with an oligonucleotide comprising a detectable label. It may be particularly advantageous to couple a detectable label to a pendant moiety of a detectable probe to facilitate dissociation of the detectable label. In some cases, a detectable label is not coupled to one, some or all pendant moieties of a nucleic acid nanoparticle. For example, fluorophore may be incorporated into internal portions of a nucleic acid nanoparticle of an anchoring moiety to decrease the likelihood of dissociation. A plurality of photodamage inhibitors may be coupled to a pendant moiety whether or not the pendant moiety is also coupled to detectable label(s).

A composition may comprise a biomolecule coupled to a nucleic acid nanostructure. In some cases, a biomolecule may be coupled to a nucleic acid nanostructure, in which the biomolecule can optionally comprise an affinity agent. For example, a detectable probe may be formed in part by coupling an affinity agent or a plurality thereof to a nucleic acid nanostructure. In some cases, a nucleic acid nanostructure may comprise two or more affinity agents, in which the two or more affinity agents are coupled to a single face of the nucleic acid nanostructure. In other cases, a nucleic acid nanostructure may comprise two or more affinity agents, in which the two or more affinity agents are coupled to two or more faces of the nucleic acid nanostructure. In some cases, a biomolecule may be coupled to a nucleic acid nanostructure, in which the biomolecule comprises a single analyte (e.g., a polysaccharide, a nucleic acid, a polypeptide, a metabolite, etc.). For example, an anchoring moiety may comprise a nucleic acid nanostructure, in which the nucleic acid nanostructure is coupled to a polypeptide analyte and a plurality of photodamage inhibitors.

In some cases, a composition may comprise a nucleic acid nanostructure and a plurality of photodamage inhibitors that are coupled to the nucleic acid nanostructure, in which photodamage inhibitors of the plurality of photodamage inhibitors are exogenous photodamage inhibitors. For example, small molecule photolabile or photoisomerization compounds may be coupled to a nucleic acid nanostructure, in which none of the small molecule photolabile or photoisomerization compounds comprise natural nucleotides. In other cases, a composition may comprise a nucleic acid nanostructure and a plurality of photodamage inhibitors that are coupled to the nucleic acid nanostructure, in which photodamage inhibitors of the plurality of photodamage inhibitors are endogenous photodamage inhibitors. For example, a nucleic acid nanostructure may comprise pendant oligonucleotides that do not serve a structural function, and can absorb photons without impairing the intended function of the nucleic acid nanostructure. In some cases, a nucleic acid nanostructure may be coupled to a similar type of biomolecule as an analyte coupled to the nucleic acid nanostructure, in which the similar type of biomolecule comprises an endogenous photodamage inhibitor. For example, a nucleic acid nanostructure coupled to a polypeptide analyte may further comprise a sacrificial polypeptide, in which the polypeptide comprises an increased concentration of amino acid residues that are likely to absorb photons (e.g., tyrosine, tryptophan, phenylalanine).

A composition may comprise a moiety (e.g., a nucleic acid nanostructure, a macromolecular structure, a detectable probe), in which the moiety comprises one or more pendant moieties. A pendant moiety may comprise a polymeric chain (e.g., polyethylene glycol, polyethylene, polypropylene, etc.) or a polymeric biomolecule (e.g., a polypeptide, an oligonucleotide, etc.). A pendant moiety comprising an oligonucleotide may comprise a nucleotide sequence that is configured to hybridize to a complementary nucleotide sequence of a nucleic acid nanostructure. A pendant moiety may further comprise photodamage inhibitors. Photodamage inhibitor may be covalently or non-covalently attached to a pendant moiety. A pendant moiety may further comprise a detectable label or a plurality thereof. A pendant moiety may comprise a coupling moiety. For example, a pendant moiety may be configured to couple an anchoring moiety comprising a nucleic acid nanostructure to an analyte binding site comprising a complementary coupling moiety. In some cases, a pendant moiety may comprise two or more segments, in which the two or more segments are joined by at least one photodamage inhibitor. Pendant moieties coupled to a moiety (e.g., a nucleic acid nanostructure, a macromolecular structure, a detectable probe) may be configured to inhibit inter-moiety or intra-moiety binding (e.g., no interstrand or intrastrand self-complementarity). In some cases, a pendant moiety may comprise passivating moieties (e.g., PEG moieties, dextran moieties, etc.), in which the passivating moieties are configured to inhibit interstrand or intrastrand interactions.

A photodamage inhibitor, as set forth herein, may be coupled to a nucleic acid nanostructure via an intercalating species. An intercalating species may comprise any species that binds to a helical groove of a double-stranded nucleic acid. Exemplary intercalating species can include berberine, ethidium bromide, proflavine, quinacrine, daunomycin, doxorubicin, thalidomide, daunorubicin, dactinomycin, and modified versions thereof. In some cases, a structure comprising a double-stranded nucleic acid (e.g., a nucleic acid nanostructure) may comprise a composition comprising an intercalating agent coupled to a photodamage inhibitor by a linking moiety. A composition comprising an intercalating agent and a photodamage inhibitor may couple the photodamage inhibitor within a distance of a nucleic acid nanostructure to which the composition is coupled. If a composition comprising an intercalating agent and a photodamage inhibitor comprises a linking moiety, the linking moiety and photodamage inhibitor may comprise a pendant moiety, as set forth herein. If a composition comprising an intercalating agent and a photodamage inhibitor does not comprise a linking moiety, a photodamage inhibitor may be coupled substantially adjacent to a face of a nucleic acid nanostructure. In some cases, photodamage inhibitors may be coupled to two or more faces of a nucleic acid nanostructure, for example when using intercalating agents, due to random or spatially variable intercalation of the intercalating agents coupled to the photodamage inhibitors.

One or more types of photodamage inhibitors may be coupled to a moiety (e.g., a nucleic acid nanostructure, a macromolecular structure, a detectable probe). A plurality of photodamage inhibitors coupled to a moiety may comprise a photon scavenger species, as set forth herein, or a reactive scavenger species, as set forth herein. In some cases, a plurality of photodamage inhibitors coupled to a moiety may comprise a photon scavenger species and a reactive scavenger species. A moiety (e.g., a nucleic acid nanostructure, a macromolecular structure, a detectable probe) may comprise at least about 1, 5, 10, 20, 50, 100, 250, 500, 1000, 5000, 10000, or more than 10000 photodamage inhibitors. Alternatively or additionally, a moiety may comprise no more than about 10000, 5000, 1000, 500, 250, 100, 50, 20, 10, 5, or less than 5 photodamage inhibitors.

In another aspect, provided herein is a composition, comprising: a) a solid support comprising an analyte binding site and an interstitial region, in which the analyte binding site comprises a coupling moiety, b) an analyte coupled to the coupling moiety of the analyte binding site, c) a detectable probe coupled to the analyte, and d) a macromolecular structure coupled to the interstitial region, in which the macromolecular structure comprises a plurality of photodamage inhibitors.

In some cases, constituent moieties of a blocking agent (e.g., macromolecular structures) may be coupled to a surface of a single-analyte array (e.g., a surface of an interstitial region, a surface of an analyte binding site). A constituent moiety of a blocking agent may comprise a macromolecular structure, in which the macromolecular structure comprises a polymer (e.g., Lipidure), a polysaccharide (e.g., a dextran), a polypeptide (e.g., a serum albumin), an oligonucleotide, or a combination thereof. In some cases, a macromolecular structure may comprise a nanoparticle (e.g., organic nanoparticles, inorganic nanoparticles). A macromolecular structure may form a covalent interaction with a surface of a solid support. In some cases, a surface of a solid support may comprise a surface-coupled reactive moiety and a macromolecular structure may comprise a surface-coupling reactive moiety, in which the surface-coupled reactive moiety forms a covalent interaction with the surface-coupling reactive moiety. A macromolecular structure may form a non-covalent interaction with a surface of a solid support. In some cases, a macromolecular structure may comprise a region of electrical charge, in which a surface of a solid support comprises a region of opposite electrical charge, and in which the region of electrical charge of the macromolecular structure forms an electrostatic interaction with the region of opposite electrical charge of the surface of the solid support.

A macromolecular structure may have a molecular weight of at least about 100 Daltons (Da), 200 Da, 300 Da, 400 Da, 500 Da, 750 Da, 1000 Da, 2000 Da, 3000 Da, 4000 Da, 5000 Da, 10000 Da, or more than 10000 Da. Alternatively or additionally, a macromolecular structure may have a molecular weight of no more than about 10000 Da, 5000 Da, 4000 Da, 3000 Da, 2000 Da, 1000 Da, 750 Da, 500 Da, 400 Da, 300 Da, 200 Da, 100 Da, or less than 100 Da.

Depending upon locations of surface defects that can facilitate orthogonal binding, macromolecular structures may bind to interstitial regions and/or analyte binding sites of a single-analyte array. Orthogonal binding sites may be distributed in a spatially random fashion on a single-analyte array. Accordingly, defect densities at analyte binding sites and/or interstitial regions may be described according to a stochastic or probabilistic distribution. For example, a defect density for analyte binding sites may be described by a Poisson-like distribution with a peak site density of 4 defects per site. In another example, an interstitial region within a radius of 10 nanometers from an edge of any given analyte binding site of a single-analyte array may have a most-probable surface defect density of 10 defects, with lower probabilities for larger or smaller defect densities. Accordingly, a quantity of macromolecular structures bound to a surface of a single-analyte array at an analyte binding site or an adjacent interstitial region may be proportional to the local defect density at or adjacent to the analyte binding site. Likewise, a quantity of macromolecular structures bound to a surface of a single-analyte array at an analyte binding site or an adjacent interstitial region may be inversely proportional to an average size of macromolecular structures (i.e., a larger macromolecular structure may block more orthogonal binding sites than a smaller one).

An analyte binding site of a single analyte array may bind about 1, 2, 3, 4, 5, 6, 7, 8, 9, 10, 15, 20, 25, 30, 40, 50, 75, 100, 500, 1000, 5000, 10000, 50000, 100000, 500000, 1000000, or more than 1000000 macromolecular structures. A plurality of analyte binding sites may have an average bound macromolecular structure density of at least about 0.1, 0.5, 1, 2, 3, 4, 5, 6, 7, 8, 9, 10, 15, 20, 25, 30, 40, 50, 75, 100, 500, 1000, 5000, 10000, 50000, 100000, 500000, 1000000, or more than 1000000 macromolecular structures per site. Alternatively or additionally, a plurality of analyte binding sites may have an average bound macromolecular structure density of no more than about 1000000, 500000, 100000, 50000, 10000, 5000, 1000, 500, 100, 75, 50, 40, 30, 25, 20, 15, 10, 9, 8, 7, 6, 5, 4, 3, 2, 1, 0.5, 0.1, or less than 0.1 macromolecular structures per site.

About 1, 2, 3, 4, 5, 6, 7, 8, 9, 10, 15, 20, 25, 30, 40, 50, 75, 100, 500, 1000, 5000, 10000, 50000, 100000, 500000, 1000000, or more than 1000000 macromolecular structures may be bound within a distance of about X nanometers of an edge of an analyte binding site (i.e., a concentric radius around an analyte binding site), where X may be at least about 1, 5, 10, 20, 50, 100, or more than 100 nanometers. Alternatively or additionally, X may be no more than about 100, 50, 20, 10, 5, 1, or less than 1 nm. An average bound macromolecular structure density within a distance of about X nanometers of an edge of an analyte binding site may be at least about 1, 2, 3, 4, 5, 6, 7, 8, 9, 10, 15, 20, 25, 30, 40, 50, 75, 100, 500, 1000, 5000, 10000, 50000, 100000, 500000, 1000000, or more than 1000000 macromolecular structures. Alternatively or additionally, an average bound macromolecular structure density within a distance of about X nanometers of an edge of an analyte binding site may be no more than about 1000000, 500000, 100000, 50000, 10000, 5000, 1000, 500, 100, 75, 50, 40, 30, 25, 20, 15, 10, 9, 8, 7, 6, 5, 4, 3, 2, 1, 0.5, 0.1, or less than 0.1 macromolecular structures.

An analyte binding site of a single-analyte array may comprise a characteristic dimension (e.g., diameter, width, length) of at least about 5 nm, 10 nm, 20 nm, 50 nm, 75 nm, 100 nm, 150 nm, 200 nm, 250 nm, 300 nm, 400 nm, 500 nm, 1000 nm, or more than 1000 nm. Alternatively or additionally, an analyte binding site of a single-analyte array may comprise a characteristic dimension of no more than about 1000 nm, 500 nm, 400 nm, 300 nm, 250 nm, 200 nm, 150 nm, 100 nm, 75 nm, 50 nm, 20 nm, 10 nm, 5 nm, or less than 5 nm. A plurality of analyte binding sites of a single-analyte array may comprise an inter-site pitch (e.g., measured centerpoint to centerpoint, measured site edge to site edge) of at least about 50 nm, 100 nm, 200 nm, 300 nm, 400 nm, 500 nm, 600 nm, 700 nm, 800 nm, 900 nm, 1 micron (μm), 1.1 μm, 1.2 μm, 1.3 μm, 1.4 μm, 1.5 μm, 2 μm, 3 μm, 4 μm, 5 μm, or more than 5 μm. Alternatively or additionally, a plurality of analyte binding sites of a single-analyte array may comprise an inter-site pitch of no more than about 5 μm, 4 μm, 3 μm, 2 μm, 1.5 μm, 1.4 μm, 1.3 μm, 1.2 μm, 1.1 μm, 1 μm, 900 nm, 800 nm, 700 nm, 600 nm, 500 nm, 400 nm, 300 nm, 200 nm, 100 nm, 50 nm, or less than 50 nm. Analyte binding site size and inter-site pitch will affect the relative array areas taken up by analyte binding sites and interstitial regions. Based upon the geometry and sizing of an array, relative quantities of macromolecular structures coupled to interstitial regions and analyte binding sites can vary. Accordingly, a density of photodamage inhibitors bound to interstitial regions and/or analyte binding sites can vary proportionally with the respective areas of interstitial regions and analyte binding sites, and the respective defect densities of interstitial regions and analyte binding sites.

In another aspect, provided herein is a composition, comprising: a) a solid support, b) an anchoring moiety, in which the anchoring moiety is coupled to the solid support, c) an analyte, in which the analyte is coupled to the anchoring moiety, and d) a pendant moiety, in which the pendant moiety comprises a plurality of molecular chains and one or more photolabile groups, in which each molecular chain of the plurality of molecular chains is linked to at least one other molecular chain of the plurality of molecular chains by a photolabile group of the one or more photolabile groups. In some cases, a pendant moiety may further comprise a plurality of detectable labels, in which detectable labels are coupled to molecular chains of a plurality of molecular chains. In particular cases, a quantity of detectable labels coupled to an anchoring moiety may be proportional to a quantity of photolabile groups coupled to the anchoring moiety. Accordingly, a quantity of cleaved photolabile groups may produce a proportional decrease in detectable signal associated with the anchoring moiety.

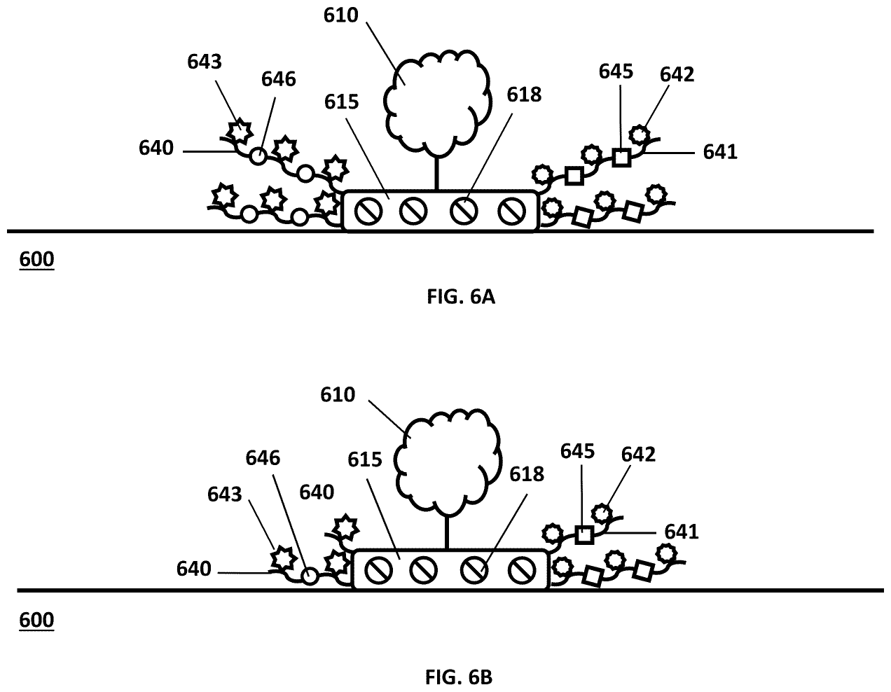

FIGS. 6A-6B depict an advantageous composition for providing photodamage protection to surface-coupled analytes, as well as providing a sensitive composition for assessing spatial distribution and extent of photodamage at a given array address. FIG. 6A depicts an analyte 610 that is coupled to an anchoring moiety 615. The anchoring moiety 615 couples the analyte 610 to a surface of a solid support 600. The anchoring moiety 615 optionally may comprise a plurality of photodamage inhibitors 618 that are coupled to the anchoring moiety 615. Coupled to the anchoring moiety 615 are a plurality of pendant moieties. In the configuration shown, the anchoring moiety 615 comprises a first plurality of pendant moieties and a second plurality of pendant moieties. Each pendant moiety of the first plurality of pendant moieties comprises a plurality of first polymeric chains 640, in which a first detectable label 643 is coupled to each first polymeric chain 640, and in which each first polymeric chain 640 is coupled to at least one other first polymeric chain 640 by a photolabile group 646. First detectable labels 643 may produce an electromagnetic signal of a first emission wavelength, and photolabile groups 646 may be cleaved by light of a scission wavelength or range of scission wavelengths. Each pendant moiety of the second plurality of pendant moieties comprises a plurality of second polymeric chains 641, in which a second detectable label 642 is coupled to each second polymeric chain 641, and in which each second polymeric chain 641 is coupled to at least one other second polymeric chain 641 by a chemically-labile group 645. Second detectable labels 642 may produce an electromagnetic signal of a second emission wavelength, and chemically-labile groups 645 may be cleaved by presence of a reactive species (e.g., a free radical species, singlet oxygen, etc.). FIG. 6B depicts the anchoring moiety of FIG. 6A after the composition has been exposed to an electromagnetic radiation field, thereby producing photodamage due to absorption of photons and presence of other photon-based species (e.g., free radicals, singlet oxygen, etc.). Three photolabile groups 646 of the first plurality of pendant moieties have been cleaved, causing the loss of 3 first polymeric chains 640 comprising 3 first detectable labels 643. Additionally, 1 chemically-labile group 645 of the second plurality of pendant moieties has been cleaved, causing the loss of 1 second polymeric chain 641 and 1 second detectable label 642. Accordingly, detection of electromagnetic signals at an array address comprising the composition of FIGS. 6A-6B would be expected to show an ˜50% decrease in signal of the first emission wavelength from the first detectable labels 643, and an ˜17% decrease in signals of the second emission wavelength from the second detectable labels 642.

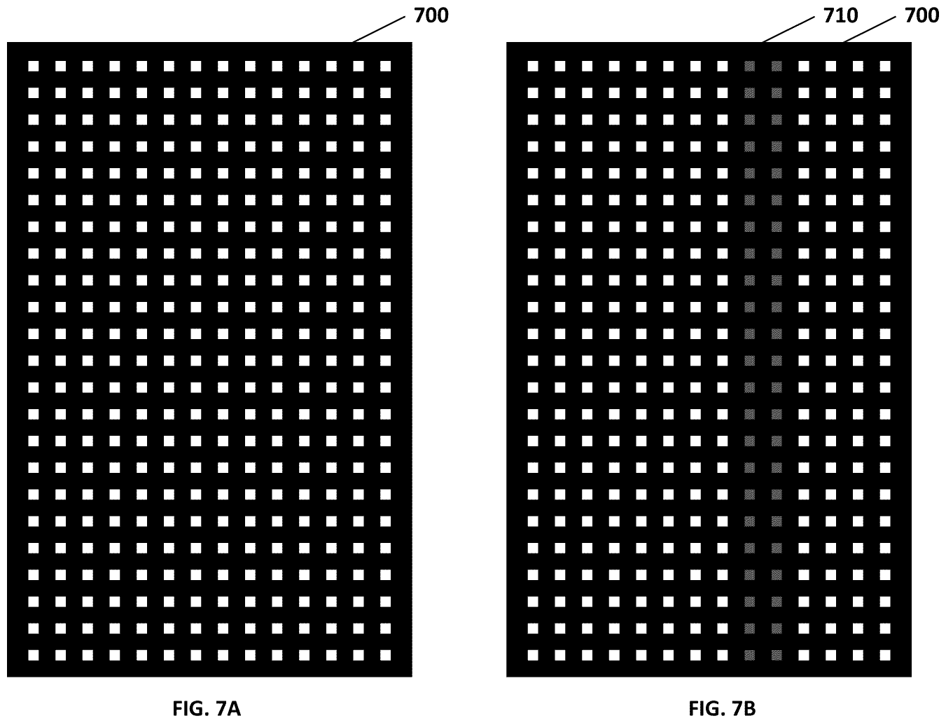

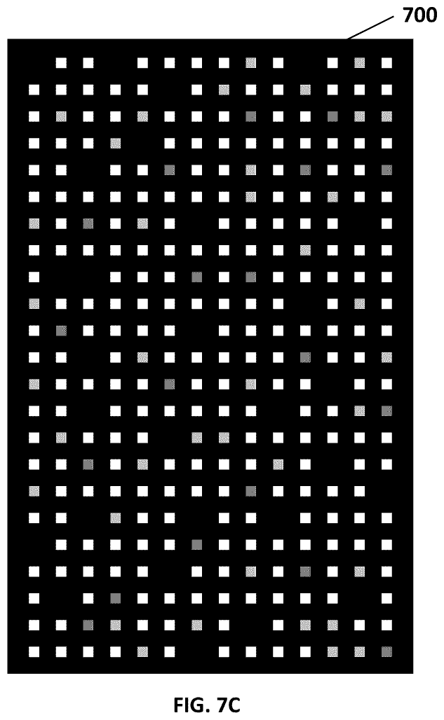

FIGS. 7A-7C illustrate use of a composition like those of FIGS. 6A-6B to identify spatial distribution and extent of photodamage on a single-analyte array. FIG. 7A depicts a simulated fluorescent image of a single-analyte array 700 with a rectangular grid of analyte binding sites. Each site is easily identifiable due to emission of signals from detectable labels, and the depicted region of the array 700 appears to have complete occupancy of all sites. FIG. 7B depicts a simulated image of the array 700 after providing from an electromagnetic radiation field at least a minimum radiative input, as set forth herein, to the array 700. In the configuration depicted in FIG. 7B, the electromagnetic radiation field (e.g., a light beam) may overlap at certain array sites as it is passed over regions of the array, thereby causing a uniform pattern of photodamage (as evidenced by decreased detectable signal) at array sites within the overlapping region. In the configuration depicted in FIG. 7C, a seemingly random spatial distribution of photodamage (as evidenced by sites with decreased or no detectable signal) is observed, thereby suggesting photodamage due to stochastic effects (e.g., random generation of reactive species) rather than a systematic effect (e.g., non-uniform EM field exposure). Compositions for assessing distribution and/or extent of photodamage may be useful for identifying array sites with decreased data confidence due to accumulated photodamage. Compositions for assessing distribution and/or extent of photodamage may also be useful for identifying systematic variations in radiative input across an array, thereby providing evidence of system malfunction, misalignment, or insufficient design.

In another aspect, provided herein is an array composition, comprising: a) a solid support comprising a plurality of analyte binding sites, in which each analyte binding site is separated from each other analyte binding site of the plurality of analyte binding sites by one or more interstitial regions, b) a plurality of analytes, in which the plurality of analytes is coupled to the plurality of sites, and in which each site of the plurality of sites comprises one and only one analyte of the plurality of analytes, c) a plurality of detectable probes, in which the plurality of detectable probes is coupled to a subset of the plurality of sites, and d) a plurality of macromolecular structures coupled to the one or more interstitial regions, in which the plurality of probes or the plurality of macromolecular structures comprise photodamage inhibitors.

A single-analyte array composition may comprise a plurality of analyte binding sites. A single-analyte array may comprise at least about 1×10², 1×10³, 1×10⁴, 1×10⁵, 1×10⁶, 1×10⁷, 1×10⁸, 1×10⁹, 1×10¹⁰, 1×10¹¹, 1×10¹², or more than 1×10¹² analyte binding sites. Alternatively or additionally, a single-analyte array may comprise no more than about 1×10¹², 1×10¹¹, 1×10¹⁰, 1×10⁹, 1×10⁸, 1×10⁷, 1×10⁶, 1×10⁵, 1×10⁴, 1×10³, 1×10², or less than 1×10² analyte binding sites.

A single-analyte array composition may be characterized by an average number of photodamage inhibitors coupled to the array. An average number of photodamage inhibitors coupled to an array may be determined before, during or after a step of providing an electromagnetic radiation field to a single-analyte array. An average number of photodamage inhibitors may be determined with respect to various array size metrics, including number of array sites, number of coupled analytes, and total or local array surface area. A single-analyte array composition may comprise at least about 0.01, 0.1, 1, 5, 10, 20, 50, 100, 250, 500, 1000, 10000, 100000, 1000000, or more than 1000000 coupled photodamage inhibitors per array site or per coupled analyte. Alternatively or additionally, a single-analyte array composition may comprise no more than about 1000000, 100000, 10000, 1000, 500, 250, 100, 50, 20, 10, 5, 1, 0.1, 0.01, or less than 0.01 coupled photodamage inhibitors per array site or per coupled analyte. A single-analyte array may comprise an average coupled photodamage inhibitor surface density of at least about 1×10⁵, 1×10⁶, 1×10⁷, 1×10⁸, 1×10⁹, 1×10¹⁰, 1×10¹¹, 1×10¹², or more than 1×10¹² coupled photodamage inhibitors per square centimeter, as determined with respect to a specific type of coupled photodamage inhibitor or all coupled photodamage inhibitors. Alternatively or additionally, a single-analyte array may comprise an average coupled photodamage inhibitor surface density of no more than about 1×10¹², 1×10¹¹, 1×10¹⁰, 1×10⁹, 1×10⁸, 1×10⁷, 1×10⁶, 1×10⁵, or less than 1×10′ coupled photodamage inhibitors per square centimeter, as determined with respect to a specific type of coupled photodamage inhibitor or all coupled photodamage inhibitors. An average number of photodamage inhibitors coupled to an array at interstitial regions and/or analyte binding sites may be determined based upon reagents contacted to an array. For example, anchoring moieties may be configured to only bind to analyte binding sites. Accordingly, photodamage inhibitors coupled to anchoring moieties would only be expected to be present at analyte binding sites. In another example, photodamage-inhibitor coupled macromolecular structures of a blocking agent may deposit at both interstitial regions and analyte binding sites, with a proportionality of binding determined by a ratio of total surface areas of the interstitial regions and analyte binding sites. Accordingly, a ratio of photodamage inhibitors coupled to interstitial regions or analyte binding sites during a surface blocking step may be proportional to the ratio of total surface areas of the interstitial regions and analyte binding sites.

Exemplary photodamage inhibitors may include photolabile species such as quinoline compounds, coumarin compounds, cyanine compounds, xanthene compounds, ortho-nitrobenzyl compounds, ortho-nitrobenzofuran compounds, benzoin compounds, BODIPY, and carbazole compounds. Additional useful photolabile compounds and linking chemistries can be found in “Photonanotechnology for Therapeutics and Imaging,” Ed. Choi, S. K., (2020); Hansen, M. J., et al., Chem. Soc. Rev., 2015, 44, 3358-3377; Piloto, A. M., et al., Tetrahedron, 2014, 70, 650-657; San Miguel, V., et al. J. Am. Chem. Soc., 2011, 133, 5380-5388; Elamri, I., et al., J. Am. Chem. Soc., 2021, 143, 10596-10603; Lv, W., et al., J. Am. Chem. Soc., 2019, 141, 17482-17486; Lu, P., et al., Matter, 2021, 4, 2172-2229; Lerch, M. M., et al., Nature Comm., 2016, 7, 12054; Hemmer, J. R., et al., J. Am. Chem. Soc., 2016, 138, 13960-13966; Sanchez-Somolinos, C., “Light-Sensitive Azobenzene-Containing Liquid Crystalline Polymers,” In Polymers and Polymeric Composities: A Reference Series, 2020, 1-31; Fedele, C., et al., “New Tricks and Emerging Applications from Contemporary Azobenzene Research,” Photochem. Photobio Sci, 2022, and Patent Cooperation Treaty Publication No. WO2012106081, each of which is incorporated by reference in its entirety. Alternatively or additionally, photodamage inhibitors may include photoisomerization species, such as stilbenes, azobenzenes, indigos, alpha-bismines, hydrazones, diarylethenes, merocyanins, spiropyrans, dihydropyrenes, retinol, thioindigo, and Stenhouse adducts. Additional aspects of photoisomerization chemistry are described in Lu, et. al., Matter, 2016, 7, 2172-2229, which is herein incorporated by reference in its entirety. Alternatively or additionally, photodamage inhibitors may include a reactive scavenger species, such as 1,3-diphenylisobenzofuran, 9,10-anthracenediyl-bis(methylene) dimalonic acid, ascorbic acid, retinol, carotenoid, or cumene. A variety of reducing agents or anti-fade agents may be used as triplet state quenchers, including, for example, ascorbic acid, dithiothreitol (DTT), mercaptoethylamine (MEA), β-mercaptoethanol (BME), n-propyl gallate, p-phenylenediamene (PPD), hydroquinone, sodium azide (NaN3), diazobicyclooctane (DABCO), cyclooctatetraene (COT), nitrobenzene, as well as commercially available anti fade agents, such as Fluoroguard (available from BioRad Laboratories, Inc., Hercules, Calif.), Citifluor antifadants (Citifluor, Ltd., London, UK), ProLong, SlowFade, and SlowFade Light (Invitrogen/Molecular Probes, Eugene, Oreg.). Likewise, a number of singlet oxygen quenchers may be used to eliminate or reduce reactive oxygen species, including, for example, enzymatic systems, e.g., superoxide dismutase, glucose oxidase/catalase (GO/Cat), oxidase/peroxidase enzyme systems, e.g., glucose oxidase, alcohol oxidases, cholesterol oxidases, lactate oxidases, pyruvate oxidases, xanthine oxidases, and the like, in combination with peroxide depleting enzymes, like horseradish peroxidase (HRP), glutathione peroxidase, or combinations of these with other enzymes, protocatachaute 3,4 dioxygenase (PCD)(a single enzyme oxygen consumer), or thiol based quenchers e.g. ergothioneine, methionine, cysteine, beta-dimethyl cysteine (penicillamine), mercaptopropionylglycine, MESNA, glutathione, dithiothreitol (as noted above for a reducing agent), N-acetyl cysteine and captopril, imidazole. Also, biological singlet oxygen quenchers may be employed such as lycopene, a, 3, and γ-carotene and their analogs, antheraxanthin, astaxanthin, canthaxanthin, neurosporene, rhodopin, bixin, norbixin, zeaxanthin, lutein, bilirubin, biliverdin, and tocopherols, as well as polyene dialdehydes, melatonin, vitamins E (α-tocopheryl succinate and its analogs) and B6 (pyridoxine 1 and its derivatives). Other chemical oxygen scavengers are also available, e.g., hydrazine (N2H4), sodium sulfite (Na2SO3), hydroxylamine, glutathione, and N-acetylcysteine, histidine, tryptophan, and the like. In addition to the foregoing, in many cases, the amount of singlet oxygen quenchers or scavengers may be reduced or eliminated by physically excluding oxygen from the reaction of interest by, e.g., degassing reagents, perfusion with inert gases, or the like. In addition to the foregoing, as an additional or alternative to the foregoing compounds, anti-oxidants may also be provided in the reaction mixture, including, e.g., Trolox and its analogs U-78715F and WIN62079, a soluble form of vitamin E, having a carboxyl substitution, or in the case of analogs, other substitutions, in place of the vitamin E phytyl side chain, ascorbic acid (or ascorbate), butylated hydroxytoluene (BTH), and the like. Additional aspects of reactive, photon-generated species chemistry can be found in Ivanov, V. E., et al. J. PhotoChem. PhotoBio. B, 2017, 176, 36-43, which is herein incorporated by reference in its entirety. Chemical or physical damage inhibitor compounds may be incorporated into a chemical sink moiety by innumerable attachment chemistries that are known in the art, such as Click-type reactions or NHS-ester chemistry onto a scaffold molecule (e.g., a polymer, a nucleic acid, etc.). Alternatively or additionally, photodamage inhibitors may include a quenching species, such as a fluorescent quencher or a dark quencher. A fluorescent quencher may include any compound that absorbs a photon of a first wavelength and subsequently emits a photon of a second wavelength. Examples of fluorescent quenchers include iodide ion, chloride ion, and acrylamide. A dark quencher may include any compound that absorbs a photon of light then releases the absorbed energy by a method other than photon emission (e.g., heat emission). Examples of dark quenchers include Black Berry quenchers (e.g., BBQ-650), Black Hole Quenchers (e.g., BHQ-1, BHQ-2, BHQ-3), dabsyl, QXL quenchers (e.g., QXL 490, QXL 570, QXL 670), Iowa Black Quenchers (e.g., Iowa Black FQ, Iowa Black RQ), and IRDye QC Quenchers (e.g., IRDye QC-1). In some cases, a photodamage inhibitor may comprise a paired quencher, such as a static quenching pair, or a dynamic quenching pair. A static quenching pair may comprise two complexed moieties that can relax absorbed photon energy non-fluorescently, such as a first quenching dye complexed with a second quenching dye, or a moiety of an analyte (e.g., an amino acid side chain) complexed with a quenching dye.

Photodamage inhibitors, as set forth herein, may be provided to a single-analyte array system as coupled photodamage inhibitors, uncoupled photodamage inhibitors, or combinations thereof. Uncoupled photodamage inhibitors may be provided in a fluidic medium, in which the uncoupled photodamage inhibitors are solvated, suspended, or otherwise mobile within the fluidic medium. Without wishing to be bound by theory, certain advantageous uncoupled photodamage inhibitors may comprise hydrophobic and/or aromatic moieties that preferentially complex or associate with aromatic or hydrophobic moieties of analytes (e.g., amino acid sidechains of amino acids like tryptophan, tyrosine, phenylalanine, valine, leucine, isoleucine, alanine, or methionine). Such uncoupled photodamage inhibitors may be provided in a sufficiently low concentration that most provided photodamage inhibitors become associated with an analyte, thereby limiting the remaining concentration of uncoupled photodamage inhibitors free in solution. Such a configuration may be advantageous for limiting photodamage to sensitive analyte moieties without substantially diminishing electromagnetic signals emitted by detectable labels.

An uncoupled photodamage inhibitor may be present in a fluidic medium at a concentration of at least about 0.001 picomolar (pM), 0.01 pM, 0.1 pM, 1 pM, 10 pM, 100 pM, 1 nanomolar (nM), 10 nM, 100 nM, 1 micromolar (pM), 10 μM, 100 μM, 1 millimolar (mM), 10 mM, 100 mM, 1 molar (M), 10 M, or more than 10 M. Alternatively or additionally, an uncoupled photodamage inhibitor may be present in a fluidic medium at a concentration of a no more than about 10 M, 1 M, 100 mM, 10 mM, 1 mM, 100 μM, 10 μM, 1 μM, 100 nM, 10 nM, 1 nM, 100 μM, 10 μM, 1 μM, 0.1 μM, 0.1 μM, 0.01 μM, 0.001 μM, or less than 0.001 μM. An uncoupled photodamage inhibitor may be provided in a total quantity that is substantially similar to (e.g., within about ±5%, 10%, 20%, 25%, 50%, 75%, 90%, 95%, 100%, 200%, 500%, 1000%, or more than 1000%) of a total quantity of protectable moieties of analytes on a single-analyte array. For example, if a single-analyte array comprised about 1 nanomole of polypeptides, with a total quantity of protectable moieties (e.g., aromatic sidechain moieties) of about 250 picomoles, it might be desirable to provide about 250 picomoles of uncoupled photodamage inhibitors to the array. If the array was contacted with 100 microliters of fluid, the fluid would preferably have a concentration of about 2.5 micromolar uncoupled photodamage inhibitors to provide the desired 250 picomoles.

### Polypeptide Assays

The present disclosure provides compositions, apparatus and methods that can be useful for characterizing sample components, such as proteins, nucleic acids, cells or other species, by obtaining multiple separate and non-identical measurements of the sample components. In particular configurations, the individual measurements may not, by themselves, be sufficiently accurate or specific to make the characterization, but an aggregation of the multiple non-identical measurements can allow the characterization to be made with a high degree of accuracy, specificity and confidence. For example, the multiple separate measurements can include subjecting the sample to reagents that are promiscuous with regard to recognizing multiple components of the sample. Accordingly, a first measurement carried out using a first promiscuous reagent may perceive a first subset of sample components without distinguishing one component from another. A second measurement carried out using a second promiscuous reagent may perceive a second subset of sample components, again, without distinguishing one component from another. However, a comparison of the first and second measurements can distinguish: (i) a sample component that is uniquely present in the first subset but not the second; (ii) a sample component that is uniquely present in the second subset but not the first; (iii) a sample component that is uniquely present in both the first and second subsets; or (iv) a sample component that is uniquely absent in the first and second subsets. The number of promiscuous reagents used, the number of separate measurements acquired, and degree of reagent promiscuity (e.g. the diversity of components recognized by the reagent) can be adjusted to suit the component diversity expected for a particular sample.

The present disclosure provides assays that are useful for detecting one or more analytes. Exemplary assays are set forth herein in the context of detecting proteins. Those skilled in the art will recognize that methods, compositions and apparatus set forth herein can be adapted for use with other analytes such as nucleic acids, polysaccharides, metabolites, vitamins, hormones, enzyme co-factors and others set forth herein or known in the art. Particular configurations of the methods, apparatus and compositions set forth herein can be made and used, for example, as set forth in U.S. Pat. No. 10,473,654 or U.S. Pat. App. Pub. Nos. 2020/0318101 A1 or 2020/0286584 A1, each of which is incorporated herein by reference. Exemplary methods, systems and compositions are set forth in further detail below.

A composition, apparatus or method set forth herein can be used to characterize an analyte, or moiety thereof, with respect to any of a variety of characteristics or features including, for example, presence, absence, quantity (e.g. amount or concentration), chemical reactivity, molecular structure, structural integrity (e.g. full length or fragmented), maturation state (e.g. presence or absence of pre- or pro-sequence in a protein), location (e.g. in an analytical system, subcellular compartment, cell or natural environment), association with another analyte or moiety, binding affinity for another analyte or moiety, biological activity, chemical activity or the like. An analyte can be characterized with regard to a relatively generic characteristic such as the presence or absence of a common structural feature (e.g. amino acid sequence length, overall charge or overall pKa for a protein) or common moiety (e.g. a short primary sequence motif or post-translational modification for a protein). An analyte can be characterized with regard to a relatively specific characteristic such as a unique amino acid sequence (e.g. for the full length of the protein or a motif), an RNA or DNA sequence that encodes a protein (e.g. for the full length of the protein or a motif), or an enzymatic or other activity that identifies a protein. A characterization can be sufficiently specific to identify an analyte, for example, at a level that is considered adequate or unambiguous by those skilled in the art.

In particular configurations, a protein can be detected using one or more affinity agents having known or measurable binding affinity for the protein. For example, an affinity agent can bind a protein to form a complex and a signal produced by the complex can be detected. A protein that is detected by binding to a known affinity agent can be identified based on the known or predicted binding characteristics of the affinity agent. For example, an affinity agent that is known to selectively bind a candidate protein suspected of being in a sample, without substantially binding to other proteins in the sample, can be used to identify the candidate protein in the sample merely by observing the binding event. This one-to-one correlation of affinity agent to candidate protein can be used for identification of one or more proteins. However, as the protein complexity (i.e. the number and variety of different proteins) in a sample increases, or as the number of different candidate proteins to be identified increases, the time and resources to produce a commensurate variety of affinity agents having one-to-one specificity for the proteins approaches limits of practicality.

Methods set forth herein, can be advantageously employed to overcome these constraints. In particular configurations, the methods can be used to identify a number of different candidate proteins that exceeds the number of affinity agents used. For example, the number of candidate proteins identified can be at least 5×, 10×, 25×, 50×, 100× or more than the number of affinity agents used. This can be achieved, for example, by (1) using promiscuous affinity agents that bind to multiple different candidate proteins suspected of being present in a given sample, and (2) subjecting the protein sample to a set of promiscuous affinity agents that, taken as a whole, are expected to bind each candidate protein in a different combination, such that each candidate protein is expected to be encoded by a unique profile of binding and non-binding events. Promiscuity of an affinity agent is a characteristic that can be understood relative to a given population of proteins. Promiscuity can arise due to the affinity agent recognizing an epitope that is known to be present in a plurality of different candidate proteins suspected of being present in the given population of unknown proteins. For example, epitopes having relatively short amino acid lengths such as dimers, trimers, or tetramers can be expected to occur in a substantial number of different proteins in the human proteome. Alternatively or additionally, a promiscuous affinity agent can recognize different epitopes (e.g. epitopes differing from each other with regard to amino acid composition or sequence), the different epitopes being present in a plurality of different candidate proteins. For example, a promiscuous affinity agent that is designed or selected for its affinity toward a first trimer epitope may bind to a second epitope that has a different sequence of amino acids when compared to the first epitope.

Although performing a single binding reaction between a promiscuous affinity agent and a complex protein sample may yield ambiguous results regarding the identity of the different proteins to which it binds, the ambiguity can be resolved when the results are combined with other identifying information about those proteins. The identifying information can include characteristics of the protein such as length (i.e. number of amino acids), hydrophobicity, molecular weight, charge to mass ratio, isoelectric point, chromatographic fractionation behavior, enzymatic activity, presence or absence of post translational modifications or the like. The identifying information can include results of binding with other promiscuous affinity agents. For example, a plurality of different promiscuous affinity agents can be contacted with a complex population of proteins, in which the plurality is configured to produce a different binding profile for each candidate protein suspected of being present in the population. In this example, each of the affinity agents can be distinguishable from the other affinity agents, for example, due to unique labeling (e.g. different affinity agents having different luminophore labels), unique spatial location (e.g. different affinity agents being located at different addresses in an array), and/or unique time of use (e.g. different affinity agents being delivered in series to a population of proteins). Accordingly, the plurality of promiscuous affinity agents produces a binding profile for each individual protein that can be decoded to identify a unique combination of epitopes present in the individual protein, and this can in turn be used to identify the individual protein as a particular candidate protein having the same or similar unique combination of epitopes. The binding profile can include observed binding events as well as observed non-binding events and this information can be evaluated in view of the expectation that particular candidate proteins produce a similar binding profile, for example, based on presence and absence of particular epitopes in the candidate proteins.

In some configurations, distinct and reproducible binding profiles may be observed for one or more unknown proteins in a sample. However, in many cases one or more binding events produces inconclusive or even aberrant results and this, in turn, can yield ambiguous binding profiles. For example, observation of binding outcome for a single-molecule binding event can be particularly prone to ambiguities due to stochasticity in the behavior of single molecules when observed using certain detection hardware. The present disclosure provides methods that provide accurate protein identification despite ambiguities and imperfections that can arise in many contexts. In some configurations, methods for identifying, quantitating or otherwise characterizing one or more proteins in a sample utilize a binding model that evaluates the likelihood or probability that one or more candidate proteins that are suspected of being present in the sample will have produced an empirically observed binding profile. The binding model can include information regarding expected binding outcomes (e.g. binding or non-binding) for binding of one or more affinity reagent with one or more candidate proteins. The information can include an a priori characteristic of a candidate protein, such as presence or absence of a particular epitope in the candidate protein or length of the candidate protein. Alternatively or additionally, the information can include empirically determined characteristics such as propensity or likelihood that the candidate protein will bind to a particular affinity reagent. Accordingly, a binding model can include information regarding the propensity or likelihood of a given candidate protein generating a false positive or false negative binding result in the presence of a particular affinity reagent, and such information can optionally be included for a plurality of affinity reagents.

Methods set forth herein can be used to evaluate the degree of compatibility of one or more empirical binding profiles with results computed for various candidate proteins using a binding model. For example, to identify an unknown protein in a sample of many proteins, an empirical binding profile for the protein can be compared to results computed by the binding model for many or all candidate proteins suspected of being in the sample. In some configurations of the methods set forth herein, identity for the unknown protein is determined based on the likelihood of the unknown protein being a particular candidate protein given the empirical binding pattern or based on the probability of a particular candidate protein generating the empirical binding pattern. Optionally a score can be determined from the measurements that are acquired for the unknown protein with respect to many or all candidate proteins suspected of being in the sample. A digital or binary score that indicates one of two discrete states can be determined. In particular configurations, the score can be non-digital or non-binary. For example, the score can be a value selected from a continuum of values such that an identity is made based on the score being above or below a threshold value. Moreover, a score can be a single value or a collection of values. Particularly useful methods for identifying proteins using promiscuous reagents, serial binding measurements and/or decoding with binding models are set forth, for example, in U.S. Pat. No. 10,473,654 U.S. Pat. App. Pub. No. 2020/0318101 A1 or Egertson et al., BioRxiv (2021), DOI: 10.1101/2021.10.11.463967, each of which is incorporated herein by reference.

The present disclosure provides compositions, apparatus and methods for detecting one or more proteins. A protein can be detected using one or more affinity agents having binding affinity for the protein. The affinity agent and the protein can bind each other to form a complex and, during or after formation, the complex can be detected. The complex can be detected directly, for example, due to a label that is present on the affinity agent or protein. In some configurations, the complex need not be directly detected, for example, in formats where the complex is formed and then the affinity agent, protein, or a label component that was present in the complex is detected.

Many protein detection methods, such as enzyme linked immunosorbent assay (ELISA), achieve high-confidence characterization of one or more protein in a sample by exploiting high specificity binding of antibodies, aptamers or other binding agents to the protein(s) and detecting the binding event while ignoring all other proteins in the sample. ELISA is generally carried out at low plex scale (e.g. from one to a hundred different proteins detected in parallel or in succession) but can be used at higher plexity. ELISA methods can be carried out by detecting immobilized binding agents and/or proteins in multiwell plates, on arrays, or on particles in microfluidic devices. Exemplary plate-based methods include, for example, the MULTI-ARRAY technology commercialized by MesoScale Diagnostics (Rockville, Maryland) or Simple Plex technology commercialized by Protein Simple (San Jose, CA). Exemplary, array-based methods include, but are not limited to those utilizing Simoa® Planar Array Technology or Simoa® Bead Technology, commercialized by Quanterix (Billerica, MA). Further exemplary array-based methods are set forth in U.S. Pat. Nos. 9,678,068; 9,395,359; 8,415,171; 8,236,574; or 8,222,047, each of which is incorporated herein by reference. Exemplary microfluidic detection methods include those commercialized by Luminex (Austin, Texas) under the trade name xMAP® technology or used on platforms identified as MAGPIX®, LUMINEX® 100/200 or FEXMAP 3D®.

Other detection methods that can also be used, for example at low plex scale, include procedures that employ SOMAmer reagents and SOMAscan assays commercialized by Soma Logic (Boulder, CO). In one configuration, a sample is contacted with aptamers that are capable of binding proteins with specificity for the amino acid sequence of the proteins. The resulting aptamer-protein complexes can be separated from other sample components, for example, by attaching the complexes to beads (or other solid support) that are removed from other sample components. The aptamers can then be isolated and, because the aptamers are nucleic acids, the aptamers can be detected using any of a variety of methods known in the art for detecting nucleic acids, including for example, hybridization to nucleic acid arrays, PCR-based detection, or nucleic acid sequencing. Exemplary methods and compositions are set forth in U.S. Pat. Nos. 7,855,054; 7,964,356; 8,404,830; 8,945,830; 8,975,026; 8,975,388; 9,163,056; 9,938,314; 9,404,919; 9,926,566; 10,221,421; 10,239,908; 10,316,321 10,221,207 or 10,392,621, each of which is incorporated herein by reference.

In some detection assays, a protein can be cyclically modified and the modified products from individual cycles can be detected. In some configurations, a protein can be sequenced by a sequential process in which each cycle includes steps of detecting the protein and removing one or more terminal amino acids from the protein. Optionally, one or more of the steps can include adding a label to the protein, for example, at the amino terminal amino acid or at the carboxy terminal amino acid. In particular configurations, a method of detecting a protein can include steps of (i) exposing a terminal amino acid on the protein; (ii) detecting a change in signal from the protein; and (iii) identifying the type of amino acid that was removed based on the change detected in step (ii). The terminal amino acid can be exposed, for example, by removal of one or more amino acids from the amino terminus or carboxyl terminus of the protein. Steps (i) through (iii) can be repeated to produce a series of signal changes that is indicative of the sequence for the protein.

In a first configuration of a cyclical protein detection method, one or more types of amino acids in the protein can be attached to a label that uniquely identifies the type of amino acid. In this configuration, the change in signal that identifies the amino acid can be loss of signal from the respective label. For example, lysines can be attached to a distinguishable label such that loss of the label indicates removal of a lysine. Alternatively or additionally, other amino acid types can be attached to other labels that are mutually distinguishable from lysine and from each other. For example, lysines can be attached to a first label and cysteines can be attached to a second label, the first and second labels being distinguishable from each other. Exemplary compositions and techniques that can be used to remove amino acids from a protein and detect signal changes are those set forth in Swaminathan et al., Nature Biotech. 36:1076-1082 (2018); or U.S. Pat. Nos. 9,625,469 or 10,545,153, each of which is incorporated herein by reference. Methods and apparatus under development by Erisyon, Inc. (Austin, TX) may also be useful for detecting proteins.

In a second configuration of a cyclical protein detection method, a terminal amino acid of a protein can be recognized by an affinity agent that is specific for the terminal amino acid or specific for a label moiety that is present on the terminal amino acid. The affinity agent can be detected on the array, for example, due to a label on the affinity agent. Optionally, the label is a nucleic acid barcode sequence that is added to a primer nucleic acid upon formation of a complex. For example, a barcode can be added to the primer via ligation of an oligonucleotide having the barcode sequence or polymerase extension directed by a template that encodes the barcode sequence. The formation of the complex and identity of the terminal amino acid can be determined by decoding the barcode sequence. Multiple cycles can produce a series of barcodes that can be detected, for example, using a nucleic acid sequencing technique. Exemplary affinity agents and detection methods are set forth in U.S. Pat. App. Pub. No. 2019/0145982 A1; 2020/0348308 A1; or 2020/0348307 A1, each of which is incorporated herein by reference. Methods and apparatus under development by Encodia, Inc. (San Diego, CA) may also be useful for detecting proteins.

Cyclical removal of terminal amino acids from a protein can be carried out using an Edman-type sequencing reaction in which a phenyl isothiocyanate reacts with a N-terminal amino group under mildly alkaline conditions (e.g. about pH 8) to form a cyclical phenylthiocarbamoyl Edman complex derivative. The phenyl isothiocyanate may be substituted or unsubstituted with one or more functional groups, linker groups, or linker groups containing functional groups. An Edman-type sequencing reaction can include variations to reagents and conditions that yield a detectable removal of amino acids from a protein terminus, thereby facilitating determination of the amino acid sequence for a protein or portion thereof. For example, the phenyl group can be replaced with at least one aromatic, heteroaromatic or aliphatic group which may participate in an Edman-type sequencing reaction, non-limiting examples including: pyridine, pyrimidine, pyrazine, pyridazoline, fused aromatic groups such as naphthalene and quinoline), methyl or other alkyl groups or alkyl group derivatives (e.g., alkenyl, alkynyl, cyclo-alkyl). Under certain conditions, for example, acidic conditions of about pH 2, derivatized terminal amino acids may be cleaved, for example, as a thiazolinone derivative. The thiazolinone amino acid derivative under acidic conditions may form a more stable phenylthiohydantoin (PTH) or similar amino acid derivative which can be detected. This procedure can be repeated iteratively for residual protein to identify the subsequent N-terminal amino acid. Many variations of Edman-type degradation have been described and may be used including, for example, a one-step removal of an N-terminal amino acid using alkaline conditions (Chang, J. Y., FEBS LETTS., 1978, 91(1), 63-68). In some cases, Edman-type reactions may be thwarted by N-terminal modifications which may be selectively removed, for example, N-terminal acetylation or formylation (e.g., see Gheorghe M. T., Bergman T. (1995) in Methods in Protein Structure Analysis, Chapter 8: Deacetylation and internal cleavage of Proteins for N-terminal Sequence Analysis. Springer, Boston, MA. doi.org/10.1007/978-1-4899-1031-8_8).

Non-limiting examples of functional groups for substituted phenyl isothiocyanate may include ligands (e.g. biotin and biotin analogs) for known receptors, labels such as luminophores, or reactive groups such as click functionalities (e.g. compositions having an azide or acetylene moiety). The functional group may be a DNA, RNA, peptide or small molecule barcode or other tag which may be further processed and/or detected.

The removal of an amino terminal amino acid using Edman-type processes can utilize at least two main steps, the first step includes reacting an isothiocyanate or equivalent with protein N-terminal residues to form a relatively stable Edman complex, for example, a phenylthiocarbamoyl complex. The second step can include removing the derivatized N-terminal amino acid, for example, via heating. The protein, now having been shortened by one amino acid, may be detected, for example, by contacting the protein with a labeled affinity agent that is complementary to the amino terminus and examining the protein for binding to the agent, or by detecting loss of a label that was attached to the removed amino acid.

Edman-type processes can be carried out in a multiplex format to detect, characterize or identify a plurality of proteins. A method of detecting a protein can include steps of (i) exposing a terminal amino acid on a protein at an address of an array; (ii) binding an affinity agent to the terminal amino acid, where the affinity agent includes a nucleic acid tag, and where a primer nucleic acid is present at the address; (iii) extending the primer nucleic acid, thereby producing an extended primer having a copy of the tag; and (iv) detecting the tag of the extended primer. The terminal amino acid can be exposed, for example, by removal of one or more amino acids from the amino terminus or carboxyl terminus of the protein. Steps (i) through (iv) can be repeated to produce a series of tags that is indicative of the sequence for the protein. The method can be applied to a plurality of proteins on the array and in parallel. Whatever the plexity, the extending of the primer can be carried out, for example, by polymerase-based extension of the primer, using the nucleic acid tag as a template. Alternatively, the extending of the primer can be carried out, for example, by ligase- or chemical-based ligation of the primer to a nucleic acid that is hybridized to the nucleic acid tag. The nucleic acid tag can be detected via hybridization to nucleic acid probes (e.g. in an array), amplification-based detections (e.g. PCR-based detection, or rolling circle amplification-based detection) or nuclei acid sequencing (e.g. cyclical reversible terminator methods, nanopore methods, or single molecule, real time detection methods). Exemplary methods that can be used for detecting proteins using nucleic acid tags are set forth in U.S. Pat. App. Pub. No. 2019/0145982 A1; 2020/0348308 A1; or 2020/0348307 A1, each of which is incorporated herein by reference.

A protein can optionally be detected based on its enzymatic or biological activity. For example, a protein can be contacted with a reactant that is converted to a detectable product by an enzymatic activity of the protein. In other assay formats, a first protein having a known enzymatic function can be contacted with a second protein to determine if the second protein changes the enzymatic function of the first protein. As such, the first protein serves as a reporter system for detection of the second protein. Exemplary changes that can be observed include, but are not limited to, activation of the enzymatic function, inhibition of the enzymatic function, attenuation of the enzymatic function, degradation of the first protein or competition for a reactant or cofactor used by the first protein. Proteins can also be detected based on their binding interactions with other molecules such as proteins, nucleic acids, nucleotides, metabolites, hormones, vitamins, small molecules that participate in biological signal transduction pathways, biological receptors or the like. For example, a protein that participates in a signal transduction pathway can be identified as a particular candidate protein by detecting binding to a second protein that is known to be a binding partner for the candidate protein in the pathway.

The presence or absence of post-translational modifications (PTM) can be detected using a composition, apparatus or method set forth herein. A PTM can be detected using an affinity agent that recognizes the PTM or based on a chemical property of the PTM. Exemplary PTMs that can be detected, identified or characterized include, but are not limited to, myristoylation, palmitoylation, isoprenylation, prenylation, farnesylation, geranylgeranylation, lipoylation, flavin moiety attachment, Heme C attachment, phosphopantetheinylation, retinylidene Schiff base formation, dipthamide formation, ethanolamine phosphoglycerol attachment, hypusine, beta-Lysine addition, acylation, acetylation, deacetylation, formylation, alkylation, methylation, C-terminal amidation, arginylation, polyglutamylation, polyglyclyation, butyrylation, gamma-carboxylation, glycosylation, glycation, polysialylation, malonylation, hydroxylation, iodination, nucleotide addition, phosphoate ester formation, phosphoramidate formation, phosphorylation, adenylylation, uridylylation, propionylation, pyrolglutamate formation, S-glutathionylation, S-nitrosylation, S-sulfenylation, S-sulfinylation, S-sulfonylation, succinylation, sulfation, glycation, carbamylation, carbonylation, isopeptide bond formation, biotinylation, carbamylation, oxidation, reduction, pegylation, ISGylation, SUMOylation, ubiquitination, neddylation, pupylation, citrullination, deamidation, elminylation, disulfide bridge formation, proteolytic cleavage, isoaspartate formation, racemization, and protein splicing.

PTMs may occur at particular amino acid residues of a protein. For example, the phosphate moiety of a particular proteoform can be present on a serine, threonine, tyrosine, histidine, cysteine, lysine, aspartate or glutamate residue of the protein. In other examples, an acetyl moiety can be present on the N-terminus or on a lysine; a serine or threonine residue can have an O-linked glycosyl moiety; an asparagine residue can have an N-linked glycosyl moiety; a proline, lysine, asparagine, aspartate or histidine amino acid can be hydroxylated; an arginine or lysine residue can be methylated; or the N-terminal methionine or at a lysine amino acid can be ubiquitinated.

In some configurations of the apparatus and methods set forth herein, one or more proteins can be detected on a solid support. For example, protein(s) can be attached to a support, the support can be contacted with detection agents (e.g. affinity agents) in solution, the agents can interact with the protein(s), thereby producing a detectable signal, and then the signal can be detected to determine the presence of the protein(s). In multiplexed versions of this approach, different proteins can be attached to different addresses in an array, and the probing and detection steps can occur in parallel. In another example, affinity agents can be attached to a solid support, the support can be contacted with proteins in solution, the proteins can interact with the affinity agents, thereby producing a detectable signal, and then the signal can be detected to determine presence, quantity or characteristics of the proteins. This approach can also be multiplexed by attaching different affinity agents to different addresses of an array.

Proteins, affinity agents or other objects of interest can be attached to a solid support via covalent or non-covalent bonds. For example, a linker can be used to covalently attach a protein or other object of interest to an array. A particularly useful linker is a structured nucleic acid particle such as a nucleic acid nanoball (e.g. a concatemeric amplicon produced by rolling circle replication of a circular nucleic acid template) or a nucleic acid origami. For example, a plurality of proteins can be conjugated to a plurality of structured nucleic acid particles, such that each protein-conjugated particle forms an address in the array. Exemplary linkers for attaching proteins, or other objects of interest, to an array or other solid support are set forth in U.S. Pat. App. Pub. No. 2021/0101930 A1, which is incorporated herein by reference.

A protein can be detected based on proximity of two or more affinity agents. For example, the two affinity agents can include two components each: a receptor component and a nucleic acid component. When the affinity agents bind in proximity to each other, for example, due to ligands for the respective receptors being on a single protein, or due to the ligands being present on two proteins that associate with each other, the nucleic acids can interact to cause a modification that is indicative of the two ligands being in proximity. Optionally, the modification can be polymerase catalyzed extension of one of the nucleic acids using the other nucleic acid as a template. As another option, one of the nucleic acids can form a template that acts as splint to position other nucleic acids for ligation to an oligonucleotide. Exemplary methods are commercialized by Olink Proteomics AB (Uppsala Sweden) or set forth in U.S. Pat. Nos. 7,306,904; 7,351,528; 8,013,134; 8,268,554 or 9,777,315, each of which is incorporated herein by reference.

A method or apparatus of the present disclosure can optionally be configured for optical detection (e.g. luminescence detection). Analytes or other entities can be detected, and optionally distinguished from each other, based on measurable characteristics such as the wavelength of radiation that excites a luminophore, the wavelength of radiation emitted by a luminophore, the intensity of radiation emitted by a luminophore (e.g. at particular detection wavelength(s)), luminescence lifetime (e.g. the time that a luminophore remains in an excited state) or luminescence polarity. Other optical characteristics that can be detected, and optionally used to distinguish analytes, include, for example, absorbance of radiation, resonance Raman, radiation scattering, or the like. A luminophore can be an intrinsic moiety of a protein or other analyte to be detected, or the luminophore can be an exogenous moiety that has been synthetically added to a protein or other analyte.

A method or apparatus of the present disclosure can use a light sensing device that is appropriate for detecting a characteristic set forth herein or known in the art. Particularly useful components of a light sensing device can include, but are not limited to, optical sub-systems or components used in nucleic acid sequencing systems. Examples of useful sub systems and components thereof are set forth in U.S. Pat. App. Pub. No. 2010/0111768 A1 or U.S. Pat. Nos. 7,329,860; 8,951,781 or 9,193,996, each of which is incorporated herein by reference. Other useful light sensing devices and components thereof are described in U.S. Pat. Nos. 5,888,737; 6,175,002; 5,695,934; 6,140,489; or 5,863,722; or U.S. Pat. Pub. Nos. 2007/007991 A1, 2009/0247414 A1, or 2010/0111768; or WO2007/123744, each of which is incorporated herein by reference. Light sensing devices and components that can be used to detect luminophores based on luminescence lifetime are described, for example, in U.S. Pat. Nos. 9,678,012; 9,921,157; 10,605,730; 10,712,274; 10,775,305; or 10,895,534, each of which is incorporated herein by reference.

Luminescence lifetime can be detected using an integrated circuit having a photodetection region configured to receive incident photons and produce a plurality of charge carriers in response to the incident photons. The integrated circuit can include at least one charge carrier storage region and a charge carrier segregation structure configured to selectively direct charge carriers of the plurality of charge carriers directly into the charge carrier storage region based upon times at which the charge carriers are produced. See, for example, U.S. Pat. Nos. 9,606,058, 10,775,305, and 10,845,308, each of which is incorporated herein by reference. Optical sources that produce short optical pulses can be used for luminescence lifetime measurements. For example, a light source, such as a semiconductor laser or LED, can be driven with a bipolar waveform to generate optical pulses with FWHM durations as short as approximately 85 ps having suppressed tail emission. See, for example, in U.S. Pat. No. 10,605,730, which is incorporated herein by reference.

For configurations that use optical detection (e.g., luminescent detection), one or more analytes (e.g., proteins) may be immobilized on a surface, and this surface may be scanned with a microscope to detect any signal from the immobilized analytes. The microscope itself may include a digital camera or other luminescence detector configured to record, store, and analyze the data collected during the scan. A luminescence detector of the present disclosure can be configured for epiluminescent detection, total internal reflection (TIR) detection, waveguide assisted excitation, or the like.

A light sensing device may be based upon any suitable technology, and may be, for example, a charged coupled device (CCD) sensor that generates pixilated image data based upon photons impacting locations in the device. It will be understood that any of a variety of other light sensing devices may also be used including, but not limited to, a detector array configured for time delay integration (TDI) operation, a complementary metal oxide semiconductor (CMOS) detector, an avalanche photodiode (APD) detector, a Geiger-mode photon counter, a photomultiplier tube (PMT), charge injection device (CID) sensors, JOT image sensor (Quanta), or any other suitable detector. Light sensing devices can optionally be coupled with one or more excitation sources, for example, lasers, light emitting diodes (LEDs), arc lamps or other energy sources known in the art.

An optical detection system can be configured for single molecule detection. For example, waveguides or optical confinements can be used to deliver excitation radiation to locations of a solid support where analytes are located. Zero-mode waveguides can be particularly useful, examples of which are set forth in U.S. Pat. Nos. 7,181,122, 7,302,146, or 7,313,308, each of which is incorporated herein by reference. Analytes can be confined to surface features, for example, to facilitate single molecule resolution. For example, analytes can be distributed into wells having nanometer dimensions such as those set forth in U.S. Pat. Nos. 7,122,482 or 8,765,359, or U.S. Pat. App. Pub. No 2013/0116153 A1, each of which is incorporated herein by reference. The wells can be configured for selective excitation, for example, as set forth in U.S. Pat. No. 8,798,414 or 9,347,829, each of which is incorporated herein by reference. Analytes can be distributed to nanometer-scale posts, such as high aspect ratio posts which can optionally be dielectric pillars that extend through a metallic layer to improve detection of an analyte attached to the pillar. See, for example, U.S. Pat. Nos. 8,148,264, 9,410,887 or 9,987,609, each of which is incorporated herein by reference. Further examples of nanostructures that can be used to detect analytes are those that change state in response to the concentration of analytes such that the analytes can be quantitated as set forth in WO 2020/176793 A1, which is incorporated herein by reference.

An apparatus or method set forth herein need not be configured for optical detection. For example, an electronic detector can be used for detection of protons or charged labels (see, for example, U.S. Pat. App. Pub. Nos. 2009/0026082 A1; 2009/0127589 A1; 2010/0137143 A1; or 2010/0282617 A1, each of which is incorporated herein by reference in its entirety). A field effect transistor (FET) can be used to detect analytes or other entities, for example, based on proximity of a field disrupting moiety to the FET. The field disrupting moiety can be due to an extrinsic label attached to an analyte or affinity agent, or the moiety can be intrinsic to the analyte or affinity agent being used. Surface plasmon resonance can be used to detect binding of analytes or affinity agents at or near a surface. Exemplary sensors and methods for attaching molecules to sensors are set forth in U.S. Pat. App. Pub. Nos. 2017/0240962 A1; 2018/0051316 A1; 2018/0112265 A1; 2018/0155773 A1 or 2018/0305727 A1; or U.S. Pat. Nos. 9,164,053; 9,829,456; 10,036,064, each of which is incorporated herein by reference.

In some configurations of the compositions, apparatus and methods set forth herein, one or more proteins can be present on a solid support, where the proteins can optionally be detected. For example, a protein can be attached to a solid support, the solid support can be contacted with a detection agent (e.g. affinity agent) in solution, the affinity agent can interact with the protein, thereby producing a detectable signal, and then the signal can be detected to determine the presence, absence, quantity, a characteristic or identity of the protein. In multiplexed versions of this approach, different proteins can be attached to different addresses in an array, and the detection steps can occur in parallel, such that proteins at each address are detected, quantified, characterized or identified. In another example, detection agents can be attached to a solid support, the support can be contacted with proteins in solution, the proteins can interact with the detection agents, thereby producing a detectable signal, and then the signal can be detected to determine the presence of the proteins. This approach can also be multiplexed by attaching different probes to different addresses of an array.

In multiplexed configurations, different proteins can be attached to different unique identifiers (e.g. addresses in an array), and the proteins can be manipulated and detected in parallel. For example, a fluid containing one or more different affinity agents can be delivered to an array such that the proteins of the array are in simultaneous contact with the affinity agent(s). Moreover, a plurality of addresses can be observed in parallel allowing for rapid detection of binding events. A plurality of different proteins can have a complexity of at least 5, 10, 100, 1×10³, 1×10⁴, 1×10⁵ or more different native-length protein primary sequences. Alternatively or additionally, a proteome, proteome subfraction or other protein sample that is analyzed in a method set forth herein can have a complexity that is at most 1×10⁵, 1×10⁴, 1×10³, 100, 10, 5 or fewer different native-length protein primary sequences. The total number of proteins of a sample that is detected, characterized or identified can differ from the number of different primary sequences in the sample, for example, due to the presence of multiple copies of at least some protein species. Moreover, the total number of proteins of a sample that is detected, characterized or identified can differ from the number of candidate proteins suspected of being in the sample, for example, due to the presence of multiple copies of at least some protein species, absence of some proteins in a source for the sample, or loss of some proteins prior to analysis.

A protein can be attached to a unique identifier using any of a variety of means. The attachment can be covalent or non-covalent. Exemplary covalent attachments include chemical linkers such as those achieved using click chemistry or other linkages known in the art or described in U.S. patent application Ser. No. 17/062,405, which is incorporated herein by reference. Non-covalent attachment can be mediated by receptor-ligand interactions (e.g. (strept)avidin-biotin, antibody-antigen, or complementary nucleic acid strands), for example, in which the receptor is attached to the unique identifier and the ligand is attached to the protein or vice versa. In particular configurations, a protein is attached to a solid support (e.g. an address in an array) via a structured nucleic acid particle (SNAP). A protein can be attached to a SNAP and the SNAP can interact with a solid support, for example, by non-covalent interactions of the DNA with the support and/or via covalent linkage of the SNAP to the support. Nucleic acid origami or nucleic acid nanoballs are particularly useful. The use of SNAPs and other moieties to attach proteins to unique identifiers such as tags or addresses in an array are set forth in U.S. Pat. App. Ser. Nos. 17/062,405 and 63/159,500, each of which is incorporated herein by reference.

The methods, compositions and apparatus of the present disclosure are particularly well suited for use with proteins. Although proteins are exemplified throughout the present disclosure, it will be understood that other analytes can be similarly used. Exemplary analytes include, but are not limited to, biomolecules, polysaccharides, nucleic acids, lipids, metabolites, hormones, vitamins, enzyme cofactors, therapeutic agents, candidate therapeutic agents or combinations thereof. An analyte can be a non-biological atom or molecule, such as a synthetic polymer, metal, metal oxide, ceramic, semiconductor, mineral, or a combination thereof.

One or more proteins that are used in a method, composition or apparatus herein, can be derived from a natural or synthetic source. Exemplary sources include, but are not limited to biological tissues, fluids, cells or subcellular compartments (e.g. organelles). For example, a sample can be derived from a tissue biopsy, biological fluid (e.g. blood, sweat, tears, plasma, extracellular fluid, urine, mucus, saliva, semen, vaginal fluid, synovial fluid, lymph, cerebrospinal fluid, peritoneal fluid, pleural fluid, amniotic fluid, intracellular fluid, extracellular fluid, etc.), fecal sample, hair sample, cultured cell, culture media, fixed tissue sample (e.g. fresh frozen or formalin-fixed paraffin-embedded) or product of a protein synthesis reaction. A protein source may include any sample where a protein is a native or expected constituent. For example, a primary source for a cancer biomarker protein may be a tumor biopsy sample or bodily fluid. Other sources include environmental samples or forensic samples.

Exemplary organisms from which proteins or other analytes can be derived include, for example, a mammal such as a rodent, mouse, rat, rabbit, guinea pig, ungulate, horse, sheep, pig, goat, cow, cat, dog, primate, non-human primate or human; a plant such as Arabidopsis thaliana, tobacco, corn, sorghum, oat, wheat, rice, canola, or soybean; an algae such as Chlamydomonas reinhardtii; a nematode such as Caenorhabditis elegans; an insect such as Drosophila melanogaster, mosquito, fruit fly, honey bee or spider; a fish such as zebrafish; a reptile; an amphibian such as a frog or Xenopus laevis; a dictyostelium discoideum; a fungi such as Pneumocystis carinii, Takifugu rubripes, yeast, Saccharamoyces cerevisiae or Schizosaccharomyces pombe; or a Plasmodium falciparum. Proteins can also be derived from a prokaryote such as a bacterium, Escherichia coli, staphylococci or Mycoplasma pneumoniae; an archae; a virus such as Hepatitis C virus, influenza virus, coronavirus, or human immunodeficiency virus; or a viroid. Proteins can be derived from a homogeneous culture or population of the above organisms or alternatively from a collection of several different organisms, for example, in a community or ecosystem.

In some cases, a protein or other biomolecule can be derived from an organism that is collected from a host organism. For example, a protein may be derived from a parasitic, pathogenic, symbiotic, or latent organism collected from a host organism. A protein can be derived from an organism, tissue, cell or biological fluid that is known or suspected of being linked with a disease state or disorder (e.g., cancer). Alternatively, a protein can be derived from an organism, tissue, cell or biological fluid that is known or suspected of not being linked to a particular disease state or disorder. For example, the proteins isolated from such a source can be used as a control for comparison to results acquired from a source that is known or suspected of being linked to the particular disease state or disorder. A sample may include a microbiome or substantial portion of a microbiome. In some cases, one or more proteins used in a method, composition or apparatus set forth herein may be obtained from a single source and no more than the single source. The single source can be, for example, a single organism (e.g. an individual human), single tissue, single cell, single organelle (e.g. endoplasmic reticulum, Golgi apparatus or nucleus), or single protein-containing particle (e.g., a viral particle or vesicle).

A method, composition or apparatus of the present disclosure can use or include a plurality of proteins having any of a variety of compositions such as a plurality of proteins composed of a proteome or fraction thereof. For example, a plurality of proteins can include solution-phase proteins, such as proteins in a biological sample or fraction thereof, or a plurality of proteins can include proteins that are immobilized, such as proteins attached to a particle or solid support. By way of further example, a plurality of proteins can include proteins that are detected, analyzed or identified in connection with a method, composition or apparatus of the present disclosure. The content of a plurality of proteins can be understood according to any of a variety of characteristics such as those set forth below or elsewhere herein.

A plurality of proteins can be characterized in terms of total protein mass. The total mass of protein in a liter of plasma has been estimated to be 70 g and the total mass of protein in a human cell has been estimated to be between 100 pg and 500 pg depending upon cells type. See Wisniewski et al. Molecular & Cellular Proteomics 13:10.1074/mcp.M113.037309, 3497-3506 (2014), which is incorporated herein by reference. A plurality of proteins used or included in a method, composition or apparatus set forth herein can include at least 1 pg, 10 pg, 100 pg, 1 ng, 10 ng, 100 ng, 1 pg, 10 pg, 100 pg, 1 mg, 10 mg, 100 mg or more protein by mass. Alternatively or additionally, a plurality of proteins may contain at most 100 mg, 10 mg, 1 mg, 100 pg, 10 pg, 1 pg, 100 ng, 10 ng, 1 ng, 100 pg, 10 pg, 1 pg or less protein by mass.

A plurality of proteins can be characterized in terms of percent mass relative to a given source such as a biological source (e.g. cell, tissue, or biological fluid such as blood). For example, a plurality of proteins may contain at least 60%, 75%, 90%, 95%, 99%, 99.9% or more of the total protein mass present in the source from which the plurality of proteins was derived. Alternatively or additionally, a plurality of proteins may contain at most 99.9%, 99%, 95%, 90%, 75%, 60% or less of the total protein mass present in the source from which the plurality of proteins was derived.

A plurality of proteins can be characterized in terms of total number of protein molecules. The total number of protein molecules in a Saccharomyces cerevisiae cell has been estimated to be about 42 million protein molecules. See Ho et al., Cell Systems (2018), DOI: 10.1016/j.cels.2017.12.004, which is incorporated herein by reference. A plurality of proteins used or included in a method, composition or apparatus set forth herein can include at least 1 protein molecule, 10 protein molecules, 100 protein molecules, 1×10⁴ protein molecules, 1×10⁶ protein molecules, 1×10⁸ protein molecules, 1×10¹⁰ protein molecules, 1 mole (6.02214076×10²³ molecules) of protein, 10 moles of protein molecules, 100 moles of protein molecules or more. Alternatively or additionally, a plurality of proteins may contain at most 100 moles of protein molecules, 10 moles of protein molecules, 1 mole of protein molecules, 1×10¹⁰ protein molecules, 1×10⁸ protein molecules, 1×10⁶ protein molecules, 1×10⁴ protein molecules, 100 protein molecules, 10 protein molecules, 1 protein molecule or less.

A plurality of proteins can be characterized in terms of the variety of full-length primary protein structures in the plurality. For example, the variety of full-length primary protein structures in a plurality of proteins can be equated with the number of different protein-encoding genes in the source for the plurality of proteins. Whether or not the proteins are derived from a known genome or from any genome at all, the variety of full-length primary protein structures can be counted independent of presence or absence of post translational modifications in the proteins. A human proteome is estimated to have about 20,000 different protein-encoding genes such that a plurality of proteins derived from a human can include up to about 20,000 different primary protein structures. See Aebersold et al., Nat. Chem. Biol. 14:206-214 (2018), which is incorporated herein by reference. Other genomes and proteomes in nature are known to be larger or smaller. A plurality of proteins used or included in a method, composition or apparatus set forth herein can have a complexity of at least 2, 5, 10, 100, 1×10³, 1×10⁴, 2×10⁴, 3×10⁴ or more different full-length primary protein structures. Alternatively or additionally, a plurality of proteins can have a complexity that is at most 3×10⁴, 2×10⁴, 1×10⁴, 1×10³, 100, 10, 5, 2 or fewer different full-length primary protein structures.

In relative terms, a plurality of proteins used or included in a method, composition or apparatus set forth herein may contain at least one representative for at least 60%, 75%, 90%, 95%, 99%, 99.9% or more of the proteins encoded by the genome of a source from which the sample was derived. Alternatively or additionally, a plurality of proteins may contain a representative for at most 99.9%, 99%, 95%, 90%, 75%, 60% or less of the proteins encoded by the genome of a source from which the sample was derived.

A plurality of proteins can be characterized in terms of the variety of primary protein structures in the plurality including transcribed splice variants. The human proteome has been estimated to include about 70,000 different primary protein structures when splice variants ae included. See Aebersold et al., Nat. Chem. Biol. 14:206-214 (2018), which is incorporated herein by reference. Moreover, the number of the partial-length primary protein structures can increase due to fragmentation that occurs in a sample. A plurality of proteins used or included in a method, composition or apparatus set forth herein can have a complexity of at least 2, 5, 10, 100, 1×10³, 1×10⁴, 7×10⁴, 1×10⁵, 1×10⁶ or more different primary protein structures. Alternatively or additionally, a plurality of proteins can have a complexity that is at most 1×10⁶, 1×10⁵, 7×10⁴, 1×10⁴, 1×10³, 100, 10, 5, 2 or fewer different primary protein structures.

A plurality of proteins can be characterized in terms of the variety of protein structures in the plurality including different primary structures and different proteoforms among the primary structures. Different molecular forms of proteins expressed from a given gene are considered to be different proteoforms. Proteoforms can differ, for example, due to differences in primary structure (e.g. shorter or longer amino acid sequences), different arrangement of domains (e.g. transcriptional splice variants), or different post translational modifications (e.g. presence or absence of phosphoryl, glycosyl, acetyl, or ubiquitin moieties). The human proteome is estimated to include hundreds of thousands of proteins when counting the different primary structures and proteoforms. See Aebersold et al., Nat. Chem. Biol. 14:206-214 (2018), which is incorporated herein by reference. A plurality of proteins used or included in a method, composition or apparatus set forth herein can have a complexity of at least 2, 5, 10, 100, 1×10³, 1×10⁴, 1×10¹, 1×10⁶, 5×10⁶, 1×10⁷ or more different protein structures. Alternatively or additionally, a plurality of proteins can have a complexity that is at most 1×10⁷, 5×10⁶, 1×10⁶, 1×10⁵, 1×10⁴, 1×10³, 100, 10, 5, 2 or fewer different protein structures.

A plurality of proteins can be characterized in terms of the dynamic range for the different protein structures in the sample. The dynamic range can be a measure of the range of abundance for all different protein structures in a plurality of proteins, the range of abundance for all different primary protein structures in a plurality of proteins, the range of abundance for all different full-length primary protein structures in a plurality of proteins, the range of abundance for all different full-length gene products in a plurality of proteins, the range of abundance for all different proteoforms expressed from a given gene, or the range of abundance for any other set of different proteins set forth herein. The dynamic range for all proteins in human plasma is estimated to span more than 10 orders of magnitude from albumin, the most abundant protein, to the rarest proteins that have been measured clinically. See Anderson and Anderson Mol Cell Proteomics 1:845-67 (2002), which is incorporated herein by reference. The dynamic range for plurality of proteins set forth herein can be a factor of at least 10, 100, 1×10³, 1×10⁴, 1×10⁶, 1 ×10⁸, 1×10¹⁰, or more. Alternatively or additionally, the dynamic range for plurality of proteins set forth herein can be a factor of at most 1×10¹⁰, 1×10⁸, 1×10⁶, 1×10⁴, 1×10³, 100, 10 or less.

A method set forth herein can be carried out in a fluid phase or on a solid phase. For fluid phase configurations, a fluid containing one or more proteins can be mixed with another fluid containing one or more affinity agents. For solid phase configurations one or more proteins or affinity agents can be attached to a solid support. One or more components that will participate in a binding event can be contained in a fluid and the fluid can be delivered to a solid support, the solid support being attached to one or more other component that will participate in the binding event.

A method of the present disclosure can be carried out at single analyte resolution. Alternatively to single-analyte resolution, a method can be carried out at ensemble-resolution or bulk-resolution. Bulk-resolution configurations acquire a composite signal from a plurality of different analytes or affinity agents in a vessel or on a surface. For example, a composite signal can be acquired from a population of different protein-affinity agent complexes in a well or cuvette, or on a solid support surface, such that individual complexes are not resolved from each other. Ensemble-resolution configurations acquire a composite signal from a first collection of proteins or affinity agents in a sample, such that the composite signal is distinguishable from signals generated by a second collection of proteins or affinity agents in the sample. For example, the ensembles can be located at different addresses in an array. Accordingly, the composite signal obtained from each address will be an average of signals from the ensemble, yet signals from different addresses can be distinguished from each other.

A composition, apparatus or method set forth herein can be configured to contact one or more proteins (e.g. an array of different proteins) with a plurality of different affinity agents. For example, a plurality of affinity agents (whether configured separately or as a pool) may include at least 2, 5, 10, 25, 50, 100, 250, 500 or more types of affinity agents, each type of affinity agent differing from the other types with respect to the epitope(s) recognized. Alternatively or additionally, a plurality of affinity agents may include at most 500, 250, 100, 50, 25, 10, 5, or 2 types of affinity agents, each type of affinity agent differing from the other types with respect to the epitope(s) recognized. Different types of affinity agents in a pool can be uniquely labeled such that the different types can be distinguished from each other. In some configurations, at least two, and up to all, of the different types of affinity agents in a pool may be indistinguishably labeled with respect to each other. Alternatively or additionally to the use of unique labels, different types of affinity agents can be delivered and detected serially when evaluating one or more proteins (e.g. in an array).

A method of the present disclosure can be performed in a multiplex format. In multiplexed configurations, different proteins can be attached to different unique identifiers (e.g. the proteins can be attached to different addresses in an array). Multiplexed proteins can be manipulated and detected in parallel. For example, a fluid containing one or more different affinity agents can be delivered to a protein array such that the proteins of the array are in simultaneous contact with the affinity agent(s). Moreover, a plurality of addresses can be observed in parallel allowing for rapid detection of binding events. A plurality of different proteins can have a complexity of at least 5, 10, 100, 1×10³, 1×10⁴, 2×10⁴, 3×10⁴ or more different native-length protein primary sequences. Alternatively or additionally, a proteome or proteome subfraction that is analyzed in a method set forth herein can have a complexity that is at most 3×10⁴, 2×10⁴, 1×10⁴, 1×10³, 100, 10, 5 or fewer different native-length protein primary sequences. The plurality of proteins can constitute a proteome or subfraction of a proteome. The total number of proteins that is detected, characterized or identified can differ from the number of different primary sequences in the sample from which the proteins are derived, for example, due to the presence of multiple copies of at least some protein species. Moreover, the total number of proteins that are detected, characterized or identified can differ from the number of candidate proteins suspected of being present, for example, due to the presence of multiple copies of at least some protein species, absence of some proteins in a source for the proteins, or loss of some proteins prior to analysis.

A particularly useful multiplex format uses an array of proteins and/or affinity agents. A polypeptide, anchoring moiety, polypeptide composite or other analyte can be attached to a unique identifier, such as an address in an array, using any of a variety of means. The attachment can be covalent or non-covalent. Exemplary covalent attachments include chemical linkers such as those achieved using click chemistry or other linkages known in the art or described in U.S. Pat. App. Pub. No. 2021/0101930 A1, which is incorporated herein by reference. Non-covalent attachment can be mediated by receptor-ligand interactions (e.g. (strept)avidin-biotin, antibody-antigen, or complementary nucleic acid strands), for example, in which the receptor is attached to the unique identifier and the ligand is attached to the protein or vice versa. In particular configurations, a protein is attached to a solid support (e.g. an address in an array) via a structured nucleic acid particle (SNAP). A protein can be attached to a SNAP and the SNAP can interact with a solid support, for example, by non-covalent interactions of the DNA with the support and/or via covalent linkage of the SNAP to the support. Nucleic acid origami or nucleic acid nanoballs are particularly useful. The use of SNAPs and other moieties to attach proteins to unique identifiers such as tags or addresses in an array are set forth in U.S. Pat. App. Pub. No. 2021/0101930 A1, which is incorporated herein by reference.

A solid support or a surface thereof may be configured to display an analyte or a plurality of analytes. A solid support may contain one or more patterned, formed, or prepared surfaces that contain at least one address for displaying an analyte. In some cases, a solid support may contain one or more patterned, formed, or prepared surfaces that contain a plurality of addresses, with each address configured to display one or more analytes. Accordingly, an array as set forth herein may comprise a plurality of analytes coupled to a solid support or a surface thereof. In some configurations, a solid support or a surface thereof may be patterned or formed to produce an ordered or patterned array of addresses. The deposition of analytes on the ordered or patterned array of addresses may be controlled by interactions between the solid support and the analytes such as, for example, electrostatic interactions, magnetic interactions, hydrophobic interactions, hydrophilic interactions, covalent interactions, or non-covalent interactions. Accordingly, the coupling of an analyte at each address of an array may produce an ordered or patterned array of analytes whose average spacing between analytes is determined based upon the tolerance of the ordering or patterning of the solid support and the size of an analyte-binding region for each address. An ordered or patterned array of analytes may be characterized as having a regular geometry, such as a rectangular, triangular, polygonal, or annular grid. In other configurations, a solid support or a surface thereof may be non-patterned or non-ordered. The deposition of analytes on the non-ordered or non-patterned array of addresses may be controlled by interactions between the solid support and the analytes, or inter-analyte interactions such as, for example, steric repulsion, electrostatic repulsion, electrostatic attraction, magnetic repulsion, magnetic attraction, covalent interactions, or non-covalent interactions.

A solid support or a surface thereof may contain one or more structures or features. A structure or feature may comprise an elevation, profile, shape, geometry, or configuration that deviates from an average elevation, profile, shape, geometry, or configuration of a solid support or surface thereof. A structure or feature may be a raised structure or feature, such as a ridge, post, pillar, or pad, if the structure or feature extends above the average elevation of a surface of a solid support. A structure or feature may be a depressed structure, such as a channel, well, pore, or hole, if the structure or feature extends below the average elevation of a surface of a solid support. A structure or feature may be an intrinsic structure or feature of a substrate (i.e., arising due to the physical or chemical properties of the substrate, or a physical or chemical mechanism of formation), such as surface roughness structures, crystal structures, or porosity. A structure or feature may be formed by a method of processing a solid support. In some configurations, a solid support or a surface may be processed by a lithographic method to form one or more structures or features. A solid support or a surface thereof may be formed by a suitable lithographic method, including, but not limited to photolithography, Dip-Pen nanolithography, nanoimprint lithography, nanosphere lithography, nanoball lithography, nanopillar arrays, nanowire lithography, immersion lithography, neutral particle lithography, plasmonic lithography, scanning probe lithography, thermochemical lithography, thermal scanning probe lithography, local oxidation nanolithography, molecular self-assembly, stencil lithography, laser interference lithography, soft lithography, magnetolithography, stereolithography, deep ultraviolet lithography, x-ray lithography, ion projection lithography, proton-beam lithography, or electron-beam lithography.

A solid support or surface may comprise a plurality of structures or features. A plurality of structures or features may comprise an ordered or patterned array of structures or features. A plurality of structures or features may comprise a non-ordered, non-patterned, or random array of structures or features. A structure or feature may have an average characteristic dimension (e.g., length, width, height, diameter, circumference, etc.) of at least about 1 nanometer (nm), 5 nm, 10 nm, 20 nm, 30 nm, 40 nm, 50 nm, 75 nm, 100 nm, 150 nm, 200 nm, 250 nm, 300 nm, 400 nm, 500 nm, 750 nm, 1000 nm, or more than 1000 nm. Alternatively or additionally, a structure or feature may have an average characteristic dimension of no more than about 1000 nm, 750 nm, 500 nm, 400 nm, 300 nm, 250 nm, 200 nm, 150 nm, 100 nm, 75 nm, 50 nm, 40 nm, 30 nm, 20 nm, 10 nm, 5 nm, 1 nm, or less than 1 nm. An array of structures or features may have an average pitch, in which the pitch is measured as the average separation between respective centerpoints of neighboring structures or features. An array may have an average pitch of at least about 1 nm, 5 nm, 10 nm, 20 nm, 30 nm, 40 nm, 50 nm, 75 nm, 100 nm, 150 nm, 200 nm, 250 nm, 300 nm, 400 nm, 500 nm, 750 nm, 1 micron (μm), 2 μm, 5 μm, 10 μm, 50 μm, 100 μm, or more than 100 μm. Alternatively or additionally, an array may have an average pitch of no more than about 100 μm, 50 μm, 10 μm, 5 μm, 1 μm, 750 nm, 500 nm, 400 nm, 300 nm, 250 nm, 200 nm, 150 nm, 100 nm, 75 nm, 50 nm, 40 nm, 30 nm, 20 nm, 10 nm, 5 nm, 1 nm, or less than 1 nm.

A solid support or a surface thereof may include a base substrate material and, optionally, one or more additional materials that are contacted or adhered with the substrate material. A solid support may comprise one or more additional materials that are deposited, coated, or inlayed onto the substrate material. Additional materials may be added to the substrate material to alter the properties of the substrate material. For example, materials may be added to alter the surface chemistry (e.g., hydrophobicity, hydrophilicity, non-specific binding, electrostatic properties), alter the optical properties (e.g., reflective properties, refractive properties), alter the electrical or magnetic properties (e.g., dielectric materials, conducting materials, electrically-insulating materials), or alter the heat transfer characteristics of the substrate material. Additional materials contacted or adhered with a substrate material may be ordered or patterned onto the substrate material to, for example, locate the additional material at addresses or locate the additional material at interstitial regions between addresses. Exemplary additional materials may include metals (e.g., gold, silver, copper, etc.), metal oxides (e.g., titanium oxide, silicon dioxide, alumina, iron oxides, etc.), metal nitrides (e.g., silicon nitride, aluminum nitride, boron nitride, gallium nitride, etc.), metal carbides (e.g., tungsten carbide, titanium carbide, iron carbide, etc.), metal sulfides (e.g., iron sulfide, silver sulfide, etc.), and organic moieties (e.g., polyethylene glycol (PEG), dextrans, chemically-reactive functional groups, etc.).

A method of the present disclosure can include the step of coupling one or more analytes to a solid support or a surface thereof prior to performing a detection step set forth herein. The coupling of one or more analytes to a solid support surface may include covalent or non-covalent coupling of the one or more analytes to the solid support. Covalent coupling of an analyte to a solid support can include direct covalent coupling of an analyte to a solid support (e.g., formation of coordination bonds) or indirect covalent coupling between a reactive functional group of the analyte and a reactive functional group that is coupled to the solid support (e.g., a CLICK-type reaction). Non-covalent coupling can include the formation of any non-covalent interaction between an analyte and a solid support, including electrostatic or magnetic interactions, or non-covalent bonding interactions (e.g., ionic bonds, van der Waals interactions, hydrogen bonding, etc.). The skilled person will readily recognize that the particular analyte and the choice of solid support can affect the selection of a coupling chemistry for the compositions and methods set forth herein.

Accordingly, a coupling chemistry may be selected based upon the criterium that it provides a sufficiently stable coupling of an analyte to a solid support for a time scale that meets or exceeds the time scale of a method as set forth herein. For example, a polypeptide identification method can require a coupling of the analyte to the solid support for a sufficient amount of time to permit a series of empirical measurements of the analyte to occur. An analyte may be continuously coupled to a solid support for an observable length of time such as, for example, at least about 1 minute, 1 hour (hr), 3 hrs, 6 hrs, 12 hrs, 1 day, 1.5 days, 2 days, 3 days, 1 week (wk), 2 wks, 3 wks, 1 month, or more. The coupling of an analyte to a solid support can occur with a solution-phase chemistry that promotes the deposition of the analyte on the solid support. Coupling of an analyte to a solid support may occur under solution conditions that are optimized for any conceivable solution property, including solution composition, species concentrations, pH, ionic strength, solution temperature, etc. Solution composition can be varied by chemical species, such as buffer type, salts, acids, bases, and surfactants. In some configurations, species such as salts and surfactants may be selected to facilitate the formation of interactions between an analyte and a solid support. Covalent coupling methods for coupling an analyte to a solid support may include species such as catalyst, initiators, and promoters to facilitate particular reactive chemistries.

Coupling of an analyte to a solid support may be facilitated by a mediating group. A mediating group may modify the properties of the analyte to facilitate the coupling. Useful mediating groups have been set forth herein (e.g., structured nucleic acid particles). In some configurations, a mediating group can be coupled to an analyte prior to coupling the analyte to a solid support. Accordingly, the mediating group may be chosen to increase the strength, control, or specificity of the coupling of the analyte to the solid support. In other configurations, a mediating group can be coupled to a solid support prior to coupling an analyte to the solid support. Accordingly, the mediating group may be chosen to provide a more favorable coupling chemistry than can be provided by the solid support alone.

### EXAMPLES

### Example 1. Photodamage Inhibitors Comprising Photolabile Compounds

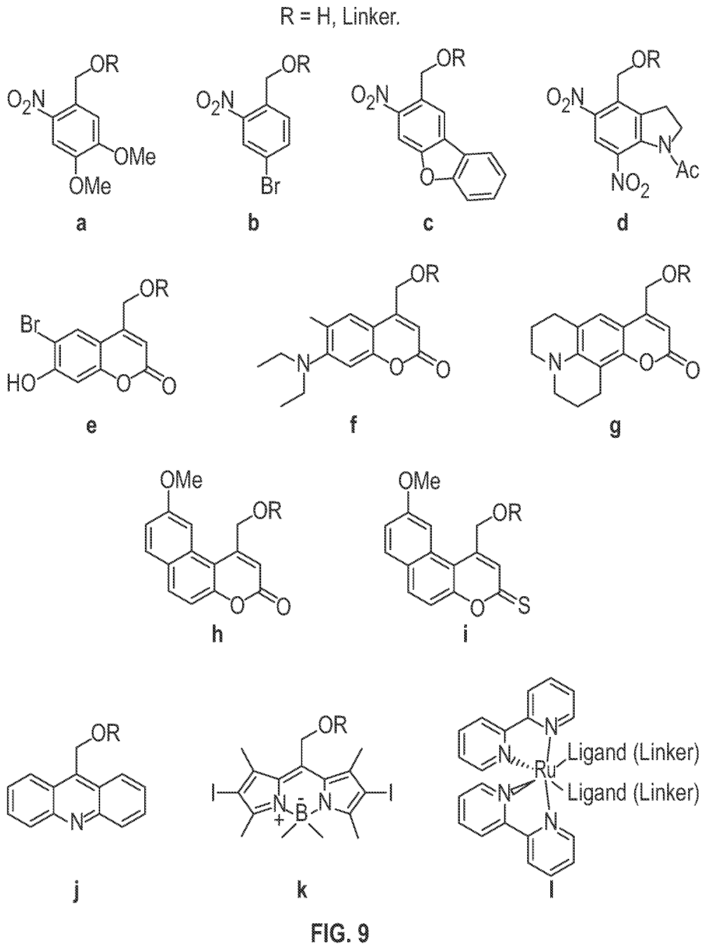

Potentially advantageous photolabile compounds are illustrated in FIG. 9. The photolabile compounds include a class of ortho-nitrobenzyl derivatives or ring fused analogues such as dibenzofuran and cyclic amine-fused compound (a-d), a class of coumarinyl-4-methyl derivatives or ring fused analogues (e-i), an acridinyl type (j), a boron-dipyrromethene (BODIPY) type (k) and a bis-bipyridine ruthenium (Ru) complex (1).

### Example 2. Representative Modes of Photon Scavenging by Photolabile Compounds

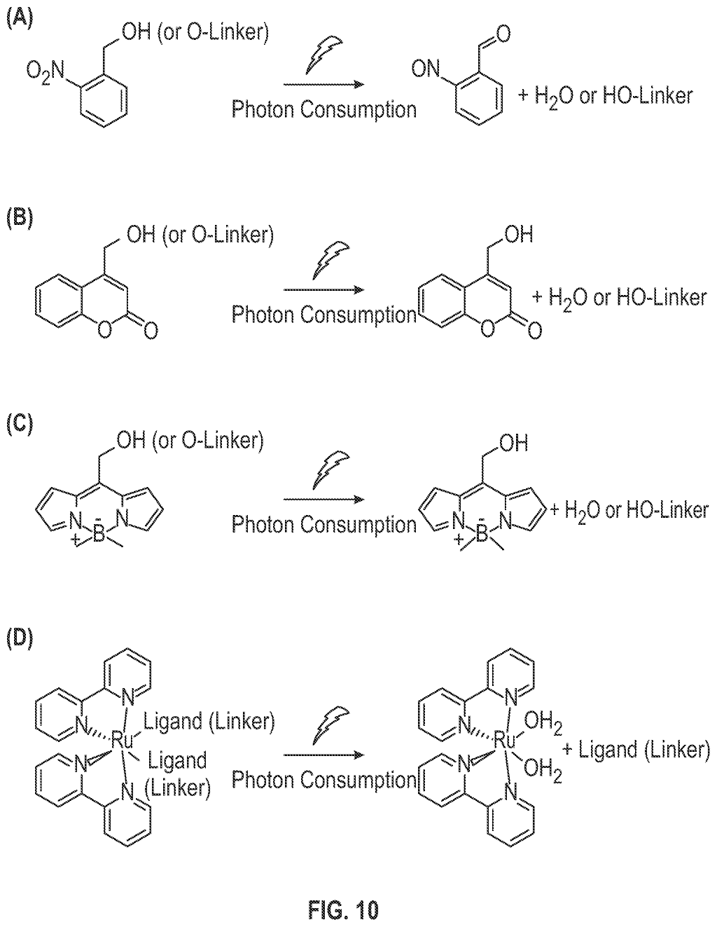

Modes of photon scavenging by photolabile compounds are illustrated in FIG. 10 with an ortho-nitrobenzyl compound (A), a coumarin-4-methyl compound (B), a boron-dipyrromethene (BODIPY) compound, (C) and a bis-bipyridine ruthenium (Ru) complex (D). Each compound displays a mechanism of photon scavenging in which its one or two photon absorption results in an excitated state which triggers its self-fragmentation through cleavage of one or more bonds. This mode of photon scavenging makes photolabile compounds consumed and released from their constructs in an irreversible manner.

### Example 3. Exemplary Synthesis of Photodamage Inhibitors Comprising Photolabile Compounds

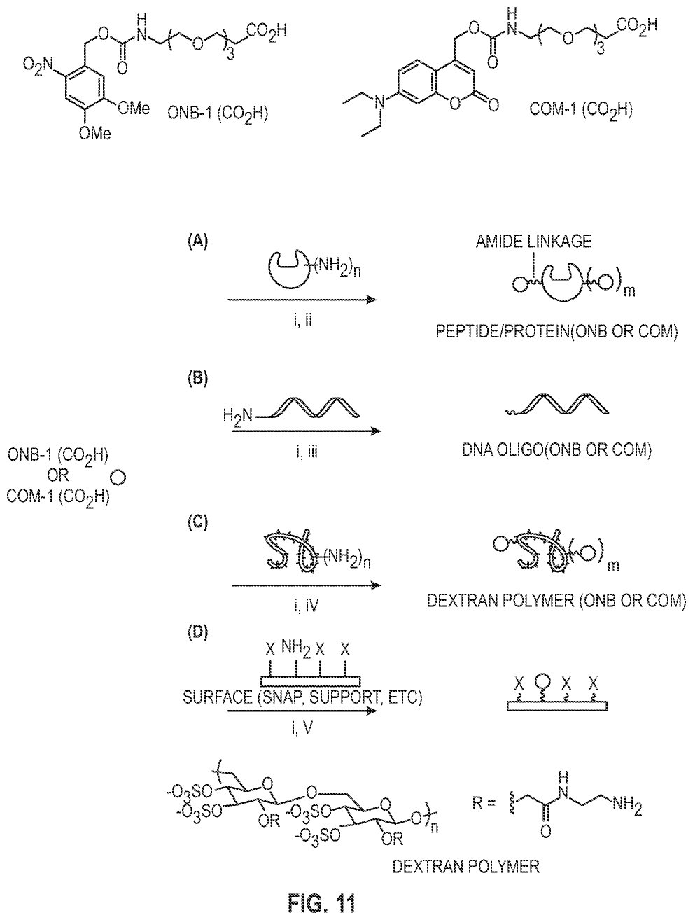

FIG. 11 provides an exemplary scheme for synthesizing assay agents comprising photolabile compounds. Each photodamage inhibitor is prepared by covalent coupling of a photolabile compound such as an ortho-nitrobenzyl (ONB), coumarinyl-4-methyl (COM) derivative and its linker construct. In this process, a carboxylic acid terminated in the linker domain of each photolabile compound ONB-1 or COM-1 is pre-activated to its activated N-hydroxysuccinimide (NHS) ester and allowed to react with an amine-presenting larger construct or macromolecule such as bovine serum albumin (A), single stranded DNA oligomer (B) and dextran sulfate polymer (C). Alternatively, an activated NHS ester can react and covalently couple to an amine residue presented on a nucleic acid nanoparticle face or to an amine moiety anchored on a solid support (D).

Reagents and conditions: (i)N-ethyl-N′-(3-dimethylaminopropyl)carbodiimide hydrochloride (EDC), N-hydroxysuccinimide (NHS), dichloromethane, 0° C. to room temperature, 12 h; then silica column purification; (ii) bovine serum albumin (BSA), 20 equiv of ONB-1 (or COM-1) NHS ester, borate-buffered saline (BBS) pH 8.4; (iii) 5′-amino-DNA oligomer (single stranded), 1.2 equiv of ONB-1 (or COM-1) NHS ester, borate-buffered saline (BBS) pH 8.4; (iv) amine-derivatized dextran sulfate, 20 equiv of ONB-1 (or COM-1) NHS ester, borate-buffered saline (BBS) pH 8.4, (v) amine-presenting SNAP or solid support, excess of ONB-1 or COM-1. Each amide conjugation is performed at room temperature overnight or for an optimal period of time determined through its reaction monitoring by high performance liquid chromatography (HPLC) or gel (SDS PAGE or agarose) analysis.

### Example 4. Photodamage Inhibitors Prepared by Covalent Coupling a Photolabile Compound to a DNA Intercalator

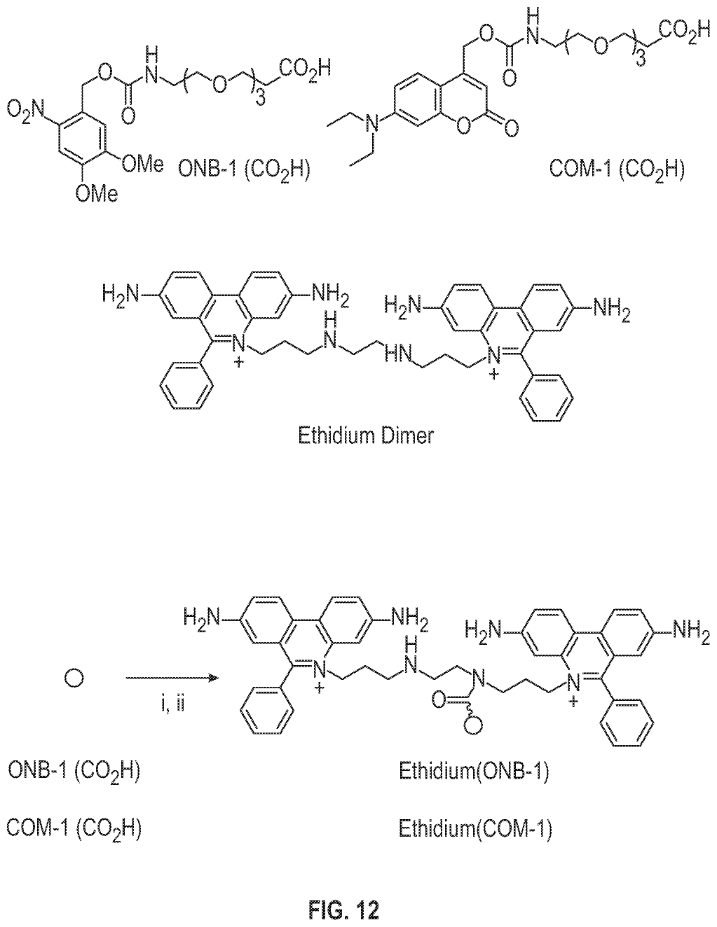

Photodamage inhibitors are prepared by covalent coupling of an ortho-nitrobenzyl (ONB) or coumarinyl-4-methyl (COM) derivatives to intercalating molecules of double stranded DNA such as an ethidium dimer molecule as presented in FIG. 12. A carboxylic acid is introduced in the linker domain of each photolabile compound, and then undergoes pre-activation to an amine-reactive NHS ester which then proceeds to conjugation with an ethidium dimer at a linker domain which contains two secondary amine residues.

Reagents and conditions: (i)N-ethyl-N′-(3-dimethylaminopropyl)carbodiimide hydrochloride (EDC), N-hydroxysuccinimide (NHS), dichloromethane, 0° C. to room temperature, 12 h; silica column purification; (ii) ethidium dimer, 1.2 equiv of ONB-1 (or COM-1) NHS ester, DMF, room temperature.

### Example 5. Photodamage Inhibitors Comprising Photoisomerization Compounds

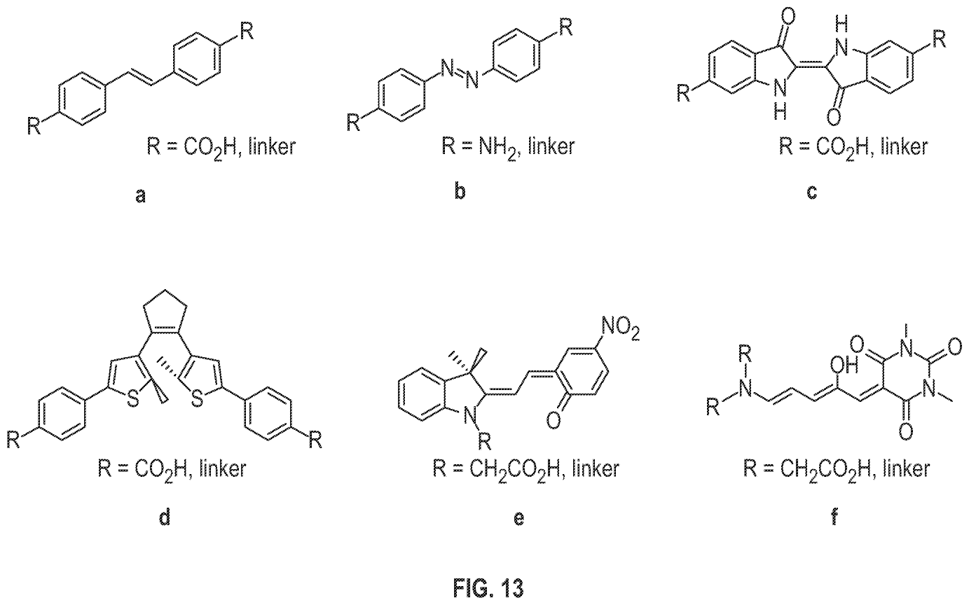

Photodamage inhibitors comprising photoisomerization compounds are illustrated in FIG. 13, including trans-stilbene and its derivatives (a), trans (E)-azobenezene and its derivatives (b), indigo and its derivatives (c), dithiopheneethene and its derivatives (d), merocyanine and its derivatives (e), a donor-acceptor Stenhouse adduct (DASA) and its derivatives (f).

### Example 6. Representative Modes of Photodamage Inhibition Through Reversible Photoisomerization

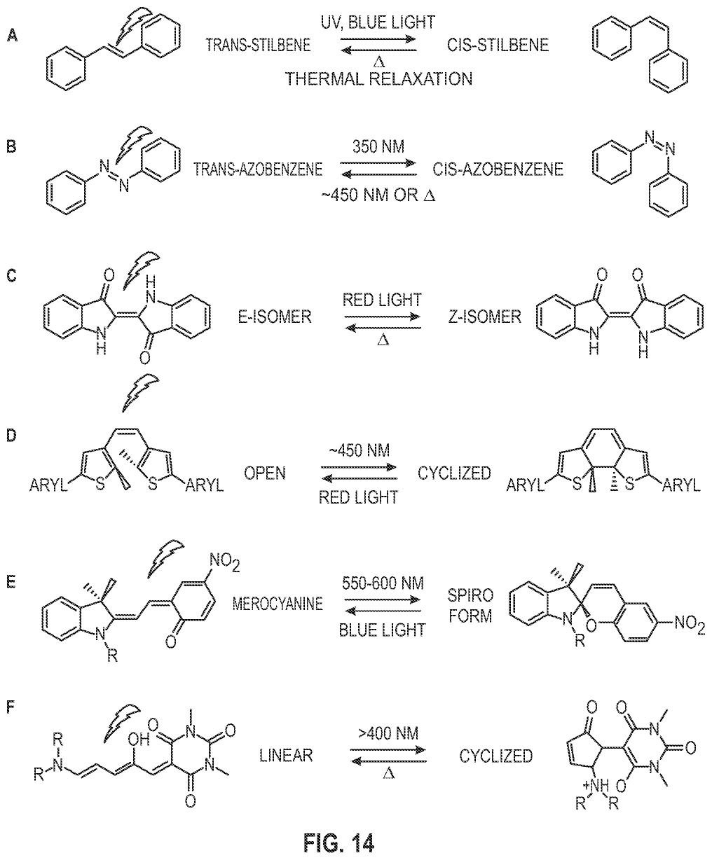

FIG. 14 illustrates various modes of photoisomerization, each associated with an isomerization system of either trans-stilbene (a), trans-azobenzene (b), indigo (c), dithiopheneethene (d), merocyanine (e) or a donor-acceptor Stenhouse adduct (f). Without wishing to be bound by theory, these photodamage inhibitors exhibit a similar mechanism of photon scavenging in which their absorption drives an isomerization from a ground state or less energetic form (e.g., trans isomer, E-isomer, ring opened form, linear conformation) to a thermodynamically excited state or more energetic form (e.g., cis isomer, Z-isomer, ring cyclized or spiro form). Once excited through light absorption, each of such isomeric forms at a higher energy state displays a mechanism of energy dissipation by undergoing isomerization back to its lower energy form through either a thermal mechanism or additional light absorption at a longer or shorter wavelength relative to the light to be scavenged. Many of these systems can undergo repeated cycles of isomerization-to-relaxation at visible light or near infrared region of light as illustrated in FIG. 14.

### Example 7. Exemplary Synthesis of Photodamage Inhibitors Comprising Photoisomerization Compounds

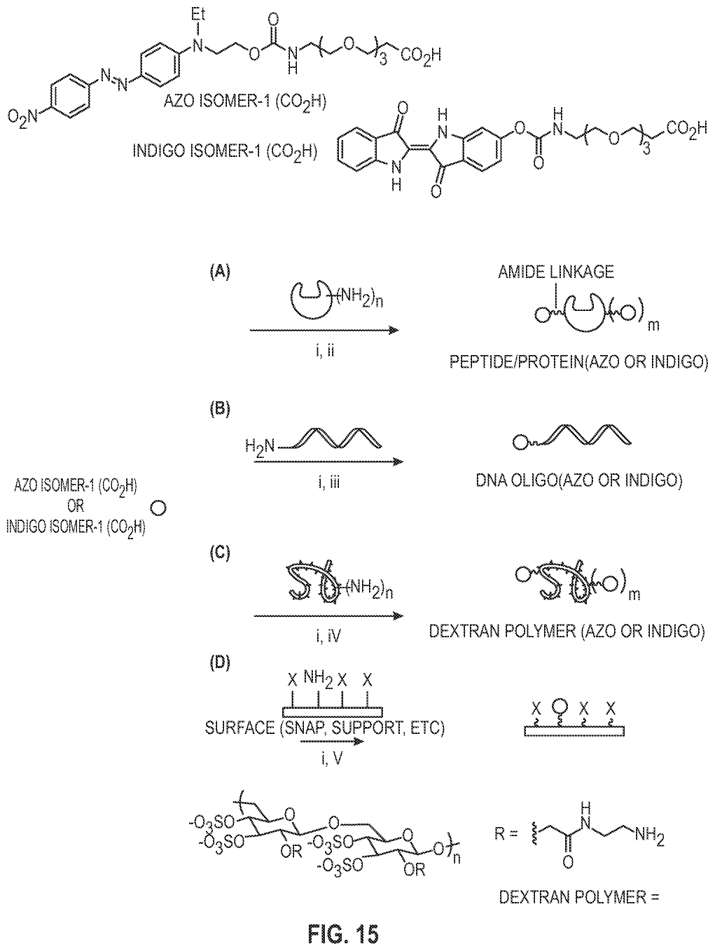

Selected examples of photoisomerization photodamage inhibitors are presented in FIG. 15. Each inhibitor is prepared by covalent coupling of an isomer such as a trans-azobenzen derivative (azo isomer-1) or E-indigo derivative (indigo isomer-1). Their synthesis involves pre-activation at a carboxylic acid terminated in the linker domain to an amine-reactive NHS ester and then reaction with an amine-presenting macromolecule such as bovine serum albumin (A), DNA oligomer (B) and dextran polymer (C). Alternatively, its activated NHS ester reacts and covalently couples to an amine residue presented on a nucleic acid nanoparticle face or an amine-terminated pendant moiety anchored on a solid support (D).

Reagents and conditions: (i)N-ethyl-N′-(3-dimethylaminopropyl)carbodiimide hydrochloride (EDC), N-hydroxysuccinimide (NHS), DMF, 0° C. to room temperature, 12 h; then silica flash column purification; (ii) bovine serum albumin (BSA), 20 equiv of azo (or indigo) isomer, borate-buffered saline (BBS) pH 8.4; (iii) 5′-amino-DNA oligomer (single stranded), 1.2 equiv of azo (or indigo) isomer, borate-buffered saline (BBS) pH 8.4; (iv) amine-derivatized dextran sulfate, 20 equiv of azo (or indigo) isomer, borate-buffered saline (BBS) pH 8.4; (v) amine-presenting SNAP or solid support, excess equiv of azo (or indigo) isomer.

### Example 8. Photodamage Inhibitors Prepared by Covalent Coupling a Photoisomerization Compound to a DNA Intercalator

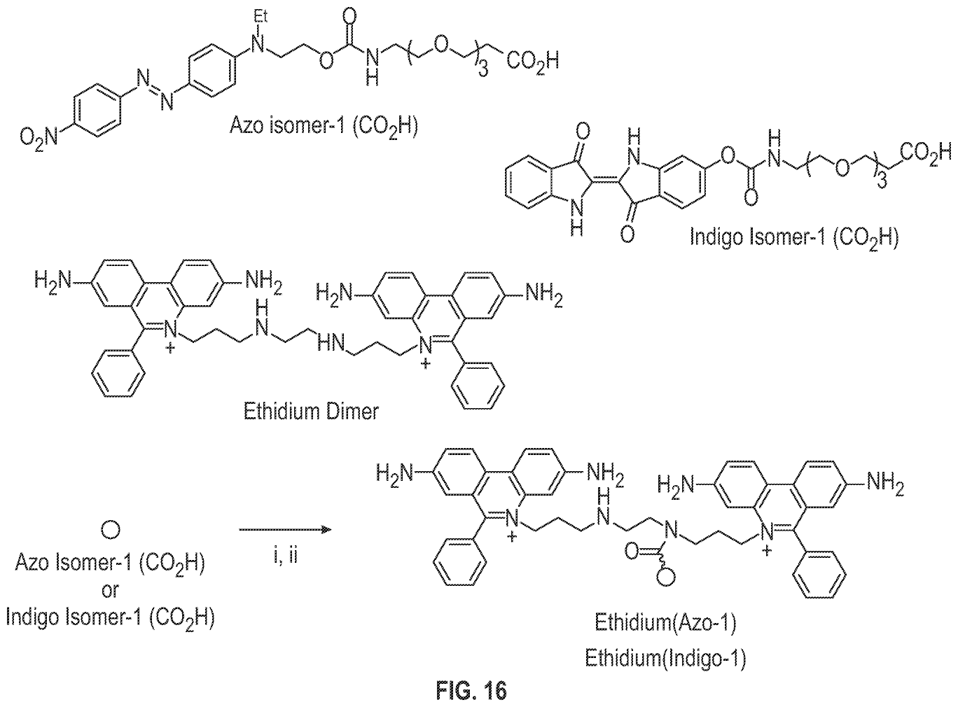

Each photoisomerization photodamage inhibitor is prepared by covalent coupling of a trans-azobenzen derivative (azo isomer-1) or E-indigo derivative (indigo isomer-1) to an ethidium dimer molecule as presented in FIG. 16. In their synthesis, a carboxylic acid introduced at the linker domain of each isomer is pre-activated to an activated NHS ester which then proceeds to reacting with an ethidium dimer at the linker domain which contains two secondary amine residues.

Reagents and conditions: (i)N-ethyl-N′-(3-dimethylaminopropyl)carbodiimide hydrochloride (EDC), N-hydroxysuccinimide (NHS), dichloromethane, 0° C. to room temperature, 24 h; silica column purification; (ii) ethidium dimer, 1.2 equiv of azo isomer-1 (or indigo isomer-1) NHS ester, DMF, room temperature, 12 h.

### Example 9. Photodamage Inhibitors Comprising Scavengers of Reactive Oxygen Species (ROS) and Free Radical Species

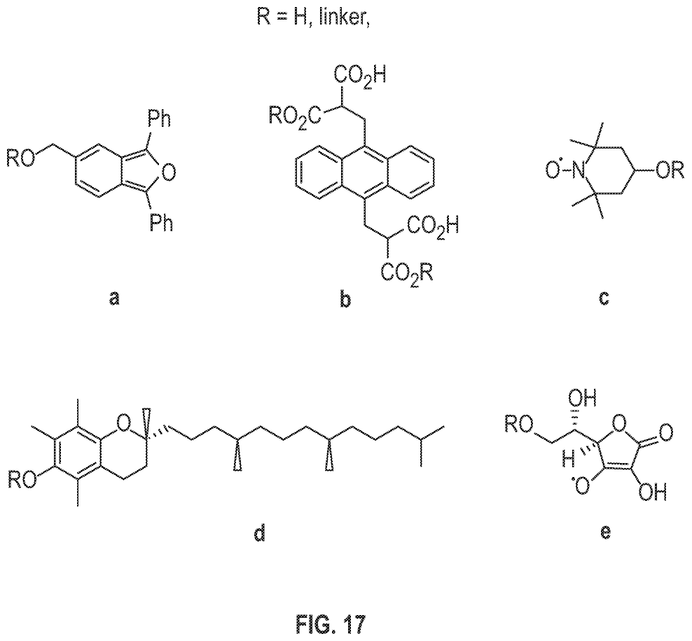

Photodamage inhibitors comprising chemical or radical scavengers are illustrated in FIG. 17 which include 1,3-diphenylisobenzofuran (DPBF) and its derivatives (a), 9,10-anthracenediyl-bis(methylene)dimalonic acid (ABDA) and its derivatives (b), 4-hydroxy-2,2,6,6-tetramethylpiperidine 1-oxyl (4-OH-TEMPO) and its derivatives (c), D-alpha-tocopherol (vitamin E) and its derivatives (d) and ascorbic acid and its derivatives (e).

### Example 10. Representative Modes of Photodamage Inhibition Through ROS and Free Radical Species Scavenging

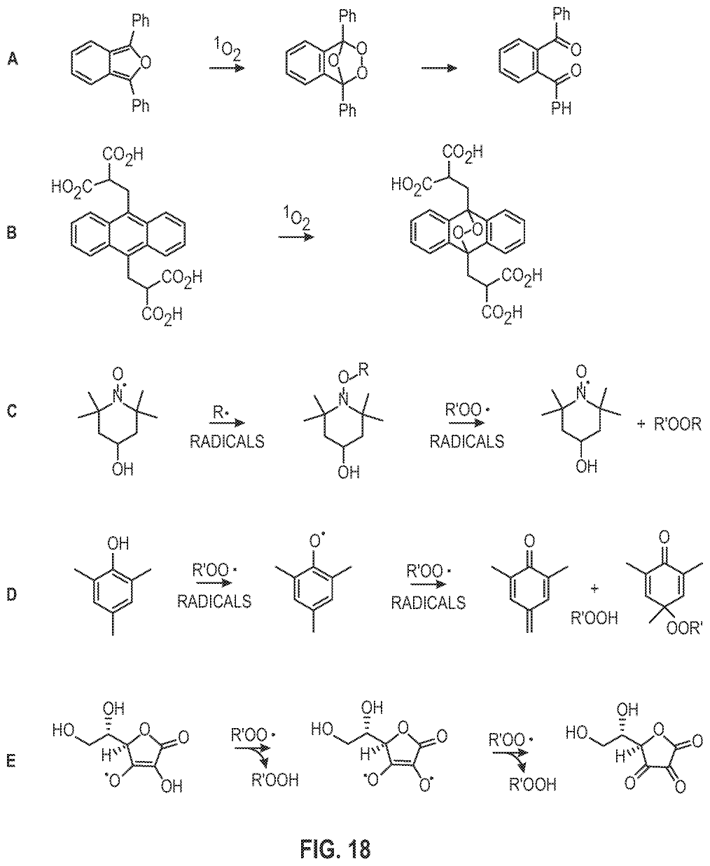

Modes of photodamage inhibition by chemical or radical scavengers are illustrated in FIG. 18, each applicable to 1,3-diphenylisobenzofuran (DPBF) (a), 9,10-anthracenediyl-bis(methylene)dimalonic acid (ABDA) (b), 4-hydroxy-2,2,6,6-tetramethylpiperidine-1-oxyl (4-OH-TEMPO) (c), 2,4,6-trimethylphenol (d) and ascorbic acid (e), respectively. Each of these scavengers comprises a set of reactive functional groups specific for irreversibly consuming or trapping ROS, nitrogen-based reactive species, or particular free radical species. Without wishing to be bound by theory, both DPBF and ABDA share a similar mechanism of reaction with singlet oxygen (10₂, an ROS member) in which one of their aromatic rings reacts with one singlet oxygen molecule to form a cyclic adduct of peroxide. Their peroxide adduct can remain stable (ABDA) or undergo a self-immolation to a more stable ketone fragment (DPPF). Other remaining scavengers including 4-OH-TEMPO, 2,4,6-trimethylphenol and ascorbic acid show certain mechanisms of scavenging or reactivity by which an ROS or free radical species is consumed and/or converted to an adduct.

### Example 11. Exemplary Synthesis of Photodamage Inhibitors Comprising ROS and Free Radical Scavengers

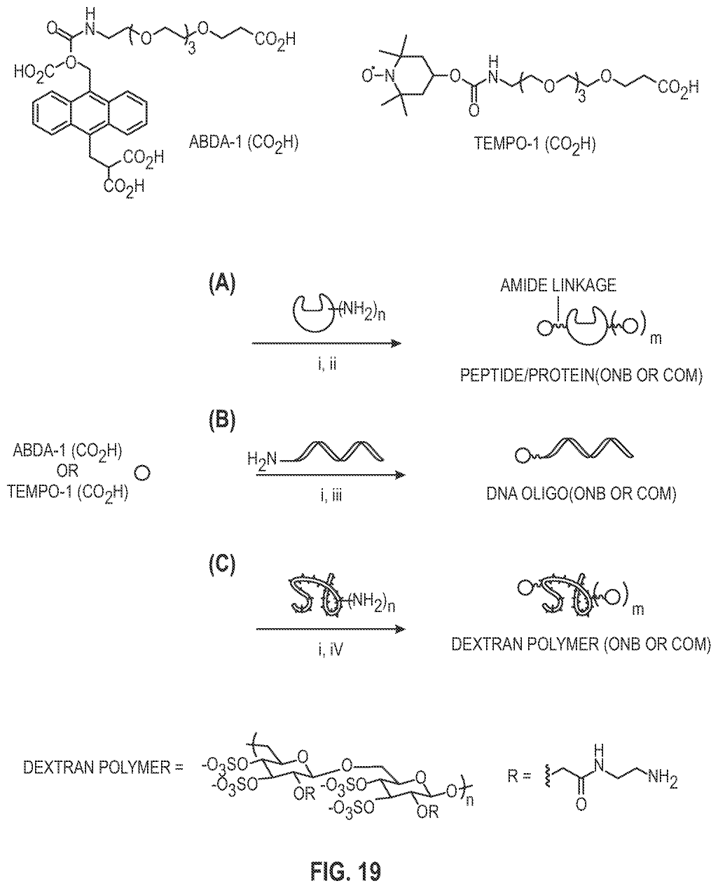

Selected examples of these inhibitors are illustrated in FIG. 19 with an ROS or free radical scavenger such as a 9,10-anthracenediyl-bis(methylene)dimalonic acid derivative (ABDA-1) and 4-hydroxy-2,2,6,6-tetramethylpiperidine-1-oxyl derivative (TEMPO-1). Each inhibitor is prepared by their covalent coupling to an assay agent, such as a polymer molecule, pendant moiety of an assay agent, or scaffold or oligonucleotide of a nucleic acid nanoparticle. Their synthesis includes pre-activation of a carboxylic acid terminated in the linker domain of each scavenger molecule to an amine-reactive NHS ester and then reacting with an amine-presenting macromolecule or assay agent, such as bovine serum albumin (A), DNA oligomer (B), or dextran sulfate polymer (C).

### Example 12. Photodamage Inhibitors Incorporated Through Azide-Alkyne Click Conjugation

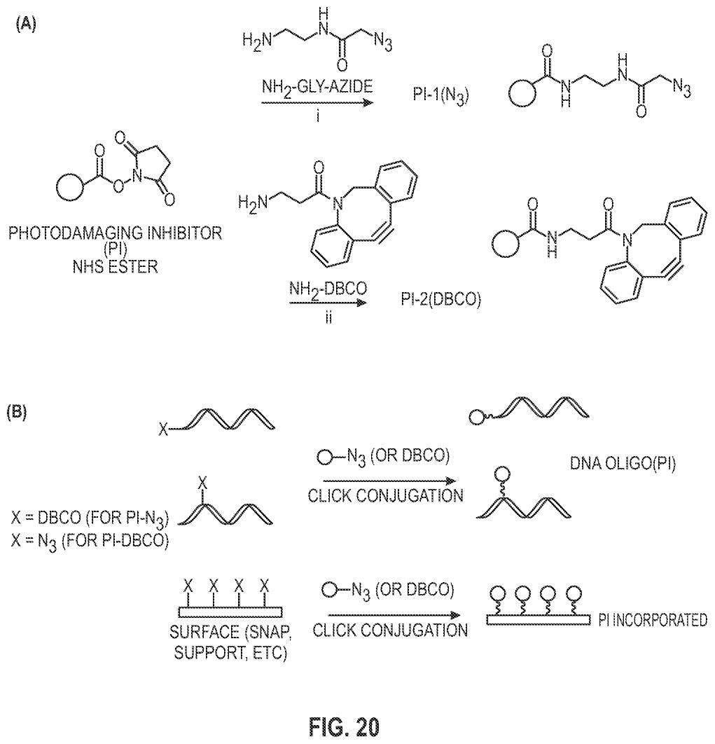

Examples of incorporated photodamage inhibitors are presented in FIG. 20. Each is functionalized with an azide or dibenzocyclooctyne (DBCO) moiety that facilitates covalent incorporation through strain-promoted azide-alkyne click (SPAAC) conjugation. In a typical process, a carboxylic acid terminated in the linker domain of a photodamage inhibitor (PI) is pre-activated to an amine-reactive NHS ester and then allowed to react with a clickable azide or dibenzocyclooctyne (DBCO) moiety, which results in PI-1 (azide) or PI-2(DBCO) as described in scheme A. Once prepared, the PI-1(azide) or PI-2 (DBCO) is incorporated in the SNAP or solid support through SPAAC conjugation as described in scheme B. This is achieved by their reaction with a clickable macromolecule or surface such as the DNA oligomer pre-modified with an azide or DBCO moiety, those moieties presented on the structured nucleic acid particle (SNAP) surface or solid support.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

Notwithstanding the appended claims, the disclosure set forth herein is also defined by the following clauses:
1) A method, comprising:
   a. providing an array comprising a plurality of sites, wherein each site of the plurality of sites comprises one and only one sample polypeptide of a plurality of sample polypeptides, wherein the array further comprises a plurality of photodamage inhibitors, and wherein photoactive agents of the plurality of photodamage inhibitors are coupled to the array;
   b. detecting each sample polypeptide of the plurality of sample polypeptides on the array at single-analyte resolution in the presence of an electromagnetic radiation field, wherein detecting each sample polypeptide on the array further comprises contacting each site of the plurality of sites with a minimum radiative input from the electromagnetic radiation field of at least 1×10⁻⁶ Joules (J); and
   c. after contacting the array with the minimum radiative input, detecting at least 90% of sample polypeptides of the plurality of sample polypeptides on the array at single-analyte resolution.
2) The method of clause 1, wherein providing the array comprises coupling the plurality of sample polypeptides to the plurality of sites.
3) The method of clause 2, wherein coupling the plurality of sample polypeptides to the plurality of sites comprises: i) coupling a single sample polypeptide of the plurality of sample polypeptides to an anchoring moiety, and ii) coupling the anchoring moiety to a site of the plurality of sites.
4) The method of clause 3, wherein the anchoring moiety is non-covalently coupled to the site of the plurality of sites.
5) The method of clause 3 or 4, wherein the anchoring moiety is covalently coupled to the site of the plurality of sites.
6) The method of any one of clauses 1-5, wherein providing the array comprises coupling the plurality of photodamage inhibitors to the array.
7) The method of clause 6, wherein coupling the plurality of photodamage inhibitors comprises coupling a first photodamage inhibitor to a site of the plurality of sites.
8) The method of clause 6 or 7, wherein coupling the plurality of photodamage inhibitors comprises coupling a second photodamage inhibitor to an interstitial region of the array.
9) The method of any one of clauses 1-8, wherein providing the array comprises coupling sample polypeptides of the plurality of sample polypeptides to the array before coupling photodamage inhibitors of the plurality of photodamage inhibitors to the array.
10) The method of any one of clauses 1-8, wherein providing the array comprises coupling sample polypeptides of the plurality of sample polypeptides to the array after coupling photodamage inhibitors of the plurality of photodamage inhibitors to the array.
11) The method of any one of clauses 1-8, wherein providing the array comprises simultaneously coupling sample polypeptides of the plurality of sample polypeptides and photodamage inhibitors of the plurality of photodamage inhibitors to the array.
12) The method of any one of clauses 6-11, wherein coupling the plurality of photodamage inhibitors to the array comprises contacting photodamage inhibitors of the plurality of photodamage inhibitors to the array.
13) The method of clause 12, wherein contacting photodamage inhibitors of the plurality of photodamage inhibitors to the array occurs in a presence of sample polypeptides of the plurality of sample polypeptides.
14) The method of clause 12, wherein contacting photodamage inhibitors of the plurality of photodamage inhibitors to the array occurs in an absence of sample polypeptides of the plurality of sample polypeptides.
15) The method of any one of clauses 1-14, wherein detecting each sample polypeptide of the plurality of sample polypeptides on the array at single-analyte resolution comprises: i) contacting a site of the plurality of sites with the electromagnetic radiation field, and ii) detecting an electromagnetic signal from the site of the plurality of sites, wherein the electromagnetic signal corresponds to a presence of a sample polypeptide of the plurality of sample polypeptides.
16) The method of any one of clauses 1-15, wherein detecting each sample polypeptide of the plurality of sample polypeptides on the array at single-analyte resolution further comprises: i) contacting the array with a first plurality of detectable probes, ii) coupling detectable probes of the first plurality of detectable probes to sample polypeptides of the plurality of sample polypeptides at a subset of sites of the plurality of sites, iii) contacting the detectable probes with the electromagnetic radiation field, and iv) at each site of a subset of the plurality of sites, detecting presence of an electromagnetic signal from a detectable probe of the detectable probes.
17) The method of clause 16, further comprising at each site of the plurality of sites, detecting a second electromagnetic signal from an anchoring moiety, wherein the anchoring moiety is coupled to the array and a sample polypeptide of the plurality of sample polypeptides.
18) The method of clause 17, wherein the electromagnetic radiation field comprises light of a first wavelength and light of a second wavelength, wherein a detectable probe of the detectable probes is configured to produce the electromagnetic signal in the presence of the light of the first wavelength, and wherein the anchoring moiety is configured to produce the second electromagnetic signal in the presence of the light of the second wavelength.
19) The method of clause 18, wherein the minimum radiative input comprises a first radiative input from the light of the first wavelength and a second radiative input from the light of the second wavelength.
20) The method of clause 19, wherein the first radiative input is at least 50% of the minimum radiative input.
21) The method of clause 19 or 20, wherein the first radiative input is no more than 90% of the minimum radiative input.
22) The method of any one of clauses 16-21, further comprising separating detectable probes from sample polypeptides at sites of the subset of the plurality of sites.
23) The method of clause 22, comprising separating detectable probes from sample polypeptides from at least 90% of sites of the subset of the plurality of sites.
24) The method of clause 22 or 23, further comprising: v) contacting the detectable probes with the electromagnetic radiation field, and vi) at each site of the at least 90% of sites of the subset of the plurality of sites, detecting an absence of an electromagnetic signal from a detectable probe of the detectable probes.
25) The method of any one of clauses 16-24, further comprising repeating steps ii)-iv).
26) The method of clause 25, wherein, before repeating steps ii)-iv), the method comprises contacting the array with the first plurality of detectable probes.
27) The method of clause 25, wherein, before repeating steps ii)-iv), the method comprises contacting the array with a second plurality of detectable probes, wherein the first plurality of detectable probes comprises affinity for a differing set of sample polypeptides than the second plurality of detectable probes.
28) The method of any one of clauses 25-27, further comprising repeating steps ii)-iv) at least ten times.
29) The method of any one of clauses 1-28, wherein contacting each site of the plurality of sites with the minimum radiative input from the electromagnetic radiation field comprises two or more cycles of contacting each site of the plurality of sites with the electromagnetic radiation field, in which the radiative input to each site of the plurality of sites during each cycle of the two or more cycles is less than the minimum radiative input.
30) The method of clause 29, further comprising, during each cycle of the two or more cycles, detecting presence or absence of an electromagnetic signal at each site of the plurality of sites.
31) The method of any one of clauses 1-30, wherein detecting at least 90% of sample polypeptides of the plurality of sample polypeptides on the array comprises detecting presence of an electromagnetic signal from at least 90% of sites of the plurality of sites.
32) The method of any one of clauses 1-31, further comprising rinsing at least a fraction of the plurality of photodamage inhibitors from the array.
33) The method of any one of clauses 1-32, further comprising contacting the array with a second plurality of photodamage inhibitors.
34) The method of clause 33, further comprising coupling at least a fraction of the second plurality of photodamage inhibitors to the array.
35) The method of clause 34, further comprising coupling no photodamage inhibitors of the plurality of photodamage inhibitors to the array.
36) The method of any one of clauses 33-35, wherein the contacting occurs before contacting each site of the plurality of sites with the minimum radiative input from the electromagnetic radiation field.
37) The method of any one of clauses 33-35, wherein the contacting occurs after contacting each site of the plurality of sites with the minimum radiative input from the electromagnetic radiation field.
38) The method of any one of clauses 1-37, further comprising contacting the array with a second plurality of photodamage inhibitors, wherein the second plurality of photodamage inhibitors is not coupled to the array.
39) A method comprising performing at least 25 cycles of an assay, wherein each cycle of the assay comprises the steps of:
   a. coupling a plurality of photodamage inhibitors to an array, wherein the array comprises a plurality of sites, wherein each site of the plurality of sites comprises one and only one sample polypeptide of a plurality of sample polypeptides, and wherein each site of the plurality of sites is resolvable at single-analyte resolution;
   b. coupling detectable probes to sample polypeptides of the plurality of sample polypeptides, wherein each probe of the detectable probes produces a detectable signal in the presence of an electromagnetic radiation field, and wherein each site of the plurality of sites receives a minimum radiative input per cycle of at least 1×10⁻⁶ Joules (J);
   c. detecting presence or absence of the detectable signal from a probe of the detectable probes at each site of the plurality of sites, wherein detecting the presence or absence of the probe comprises providing the electromagnetic radiation field; and
   d. after providing the electromagnetic radiation field, rinsing at least a fraction of the plurality of photodamage inhibitors from the array.
40) The method of clause 39, wherein detectable signals from detectable probes are detected from at least 80% of sites of the plurality of sites during the final 10 cycles of the at least 25 cycles.
41) The method of clause 40, wherein detectable signals from detectable probes are detected from at least 80% of sites of the plurality of sites during the final 5 cycles of the at least 25 cycles.
42) The method of clause 40, wherein detectable signals from detectable probes are detected from at least 90% of sites of the plurality of sites during the final 10 cycles of the at least 25 cycles.
43) The method of any one of clauses 39-42, wherein the at least 25 cycles comprises at least 50 cycles.
44) The method of clause 43, wherein the at least 25 cycles comprises at least 100 cycles.
45) The method of any one of clauses 39-44, wherein the detectable signal is detected at a site of the plurality of sites during at least 2 cycles of the at least 25 cycles of the assay.
46) The method of clause 45, wherein the detectable signal is detected at a site of the plurality of sites during at least 5 cycles of the at least 25 cycles of the assay.
47) The method of clause 45, wherein the detectable signal is detected at a site of the plurality of sites during no more than 20 cycles of the at least 25 cycles of the assay.
48) The method of any one of clauses 39-47, wherein rinsing at least the fraction of photodamage inhibitors from the array comprises flowing a fluidic medium over the array.
49) The method of clause 48, wherein the fluidic medium comprises photodamage inhibitors.
50) The method of clause 49, wherein step d) of cycle N occurs simultaneously with step a) of cycle N+1.
51) A method, comprising:
   a. providing an array comprising a plurality of sites, wherein each site of the plurality of sites comprises one and only one sample polypeptide of a plurality of sample polypeptides, and wherein each site of the plurality of sites is resolvable at single-analyte resolution;
   b. coupling detectable probes to sample polypeptides of the plurality of sample polypeptides, wherein each detectable probe comprises a fluorescent moiety that produces a detectable signal in the presence of an electromagnetic radiation field, wherein each detectable probe comprises a nucleic acid nanostructure, and wherein each detectable probe further comprises a photodamage inhibitor;
   c. at each site of the plurality of sites, detecting presence or absence of the detectable signal, wherein detecting the presence or absence of the detectable signal comprises providing the electromagnetic radiation field;
   d. after detecting the presence or absence of the detectable signal, separating the detectable probes from the sample polypeptides; and
   e. after separating the detectable probes from the sample polypeptides, detecting absence of the detectable signal at each site of the plurality of sites.
52) The method of clause 51, further comprising coupling the photodamage inhibitors to the detectable probes.
53) The method of clause 52, comprising covalently coupling the photodamage inhibitors to the detectable probes.
54) The method of clause 52, comprising non-covalently coupling the photodamage inhibitors to the detectable probes.
55) A method, comprising:
   a. providing an array comprising a plurality of sites, wherein each site of the plurality of sites comprises one and only one sample polypeptide of a plurality of sample polypeptides, wherein each site further comprises an anchoring moiety, wherein the anchoring moiety comprises a nucleic acid nanostructure, wherein the anchoring moiety couples the one and only sample polypeptide to the array, wherein the anchoring moiety further comprises a photodamage inhibitor, and wherein each site of the plurality of sites is resolvable at single-analyte resolution;
   b. contacting the array with an electromagnetic radiation field at least two times;
   c. after contacting the array with an electromagnetic radiation field at least two times, detecting at each site of the plurality of sites the presence of one and only one polypeptide of the plurality of sample polypeptides.
56) The method of clause 51, further comprising coupling photodamage inhibitors to the anchoring moieties.
57) The method of clause 52, comprising covalently coupling the photodamage inhibitors to the anchoring moieties.
58) The method of clause 52, comprising non-covalently coupling the photodamage inhibitors to the anchoring moieties.
59) A composition, comprising:
   a. a nucleic acid nanostructure, wherein the nucleic acid nanostructure comprises a first face and a second face, in which the first face and second face comprise differing average orientations;
   b. a biomolecule covalently coupled to the first face of the nucleic acid nanostructure; and
   c. a plurality of photodamage inhibitors coupled to the second face of the nucleic acid nanostructure.
60) The composition of clause 59, wherein an average orientation of the first face is rotated at least 45° from an average orientation of the second face.
61) The composition of clause 59 or 60, wherein an average orientation of the first face is rotated no more than 180° from an average orientation of the second face.
62) The composition of any one of clauses 59-61, wherein the biomolecule is substantially occluded from contacting the second face of the nucleic acid nanostructure.
63) The composition of any one of clauses 59-62, wherein the biomolecule comprises an affinity agent.
64) The composition of clause 63, wherein the affinity agent is selected from a group consisting of antibodies or functional fragments thereof (e.g., Fab′ fragments, F(ab′)₂ fragments, single-chain variable fragments (scFv), di-scFv, tri-scFv, or microantibodies), affibodies, affilins, affimers, affitins, alphabodies, anticalins, avimers, DARPins, monobodies, nanoCLAMPs, nucleic acid aptamers, protein aptamers, lectins, and functional fragments thereof.
65) The composition of clause 63 or 64, comprising two or more affinity agents.
66) The composition of clause 65, wherein each affinity agent of the two or more affinity agents is coupled to the first face.
67) The composition of any one of clauses 59-66, wherein photodamage inhibitors of the plurality of photodamage inhibitors are exogenous photodamage inhibitors.
68) The composition of any one of clauses 59-67, wherein photodamage inhibitors of the plurality of photodamage inhibitors are endogenous photodamage inhibitors.
69) The composition of any one of clauses 59-68, wherein the nucleic acid nanostructure comprises a pendant moiety.
70) The composition of clause 69, wherein the pendant moiety comprises photodamage inhibitors of the plurality of photodamage inhibitors.
71) The composition of clause 69 or 70, wherein the pendant moiety comprises a detectable label.
72) The composition of any one of clauses 69-71, wherein the pendant moiety comprises a coupling moiety.
73) The composition of any one of clauses 69-72, wherein the pendant moiety comprises a polymeric chain.
74) The composition of clause 73, wherein photodamage inhibitors are covalently coupled to the polymeric chain.
75) The composition of clause 73 or 74, wherein the polymeric chain comprises two or more chain segments, and wherein the two or more chain segments are joined by at least one photodamage inhibitor.
76) The composition of any one of clauses 73-75, wherein the polymeric chains comprises a hydrocarbon chain.
77) The composition of any one of clauses 69-76, wherein the pendant moiety comprises an oligonucleotide, wherein the oligonucleotide couples the pendant moiety to the nucleic acid nanostructure.
78) The composition of any one of clauses 69-78, wherein the nucleic acid nanostructure comprises a first pendant moiety and a second pendant moiety.
79) The composition of clause 78, wherein the first pendant moiety is not configured to bind to the second pendant moiety.
80) The composition of any one of clauses 59-79, wherein the nucleic acid nanostructure comprises a nucleic acid origami.
81) The composition of any one of clauses 59-80, wherein a photodamage inhibitor of the plurality of photodamage inhibitors is coupled to an intercalating agent, wherein the intercalating agent is non-covalently coupled to the nucleic acid nanostructure.
82) The composition of any one of clauses 59-81, wherein the plurality of photodamage inhibitors comprises a photon scavenger species or a reactive scavenger species.
83) The composition of clause 82, wherein the plurality of photodamage inhibitors comprises a photon scavenger species and a reactive scavenger species.
84) The composition of clause 82 or 83, wherein the plurality of photodamage inhibitors comprises at least 5 photodamage inhibitor moieties.
85) A composition, comprising:
   a. a solid support comprising an analyte binding site and an interstitial region, wherein the analyte binding site comprises a coupling moiety;
   b. an analyte coupled to the coupling moiety of the analyte binding site;
   c. a detectable probe coupled to the analyte; and
   d. a macromolecular structure coupled to the interstitial region;
wherein the macromolecular structure comprises a plurality of photodamage inhibitors.
86) The composition of clause 85, wherein the macromolecular structure comprises a polymer, a polysaccharide, a polypeptide, an oligonucleotide, or a combination thereof.
87) The composition of clause 85 or 86, wherein the macromolecular structure comprises a nanoparticle.
88) The composition of any one of clauses 85-87, wherein the macromolecular structure is non-covalently coupled to the interstitial region.
89) The composition of clause 88, wherein the macromolecular structure comprises a region of electrical charge, wherein the interstitial region comprises a region of opposite electrical charge, and wherein the region of electrical charge of the macromolecular structure is electrostatically coupled to the region of opposite electrical charge of the interstitial region.
90) The composition of any one of clauses 85-87, wherein the macromolecular structure is covalently coupled to the interstitial region.
91) The composition of clause 90, wherein the interstitial region comprises a surface-coupled moiety, wherein the macromolecular structure is covalently coupled to the surface-coupled moiety.
92) The composition of any one of clauses 85-91, wherein the macromolecular structure comprises a molecular weight of at least 500 Daltons (Da).
93) The composition of any one of clauses 85-92, wherein the macromolecular structure comprises at least 5 photodamage inhibitor moieties.
94) The composition of any one of clauses 85-93, wherein the macromolecular structure comprises a plurality of photodamage inhibitors, wherein the plurality of photodamage inhibitors comprises a photon scavenger species or a reactive scavenger species.
95) The composition of any one of clauses 85-94, wherein the analyte binding site comprises a characteristic dimension of at least 50 nanometers (nm).
96) The composition of clause 95, wherein the analyte binding site comprises a characteristic dimension of no more than 500 nanometers (nm).
97) The composition of clause 95 or 96, wherein the macromolecular structure is coupled to the interstitial region within a distance to the analyte binding site of no more than 10 nanometers.
98) The composition of any one of clauses 85-97, wherein two or more macromolecular structures are coupled to the array.
99) The composition of clause 98, wherein the two or more macromolecular structures are coupled to the array within an average distance to the analyte binding site of no more than 10 nanometers.
100) An array composition, comprising:
   a. a solid support comprising a plurality of analyte binding sites, wherein each analyte binding site is separated from each other analyte binding site of the plurality of analyte binding sites by one or more interstitial regions;
   b. a plurality of analytes, wherein the plurality of analytes is coupled to the plurality of sites, and wherein each site of the plurality of sites comprises one and only one analyte of the plurality of analytes;
   c. a plurality of detectable probes, wherein the plurality of detectable probes is coupled to a subset of the plurality of sites; and
   d. a plurality of macromolecular structures coupled to the one or more interstitial regions;
wherein the plurality of probes or the plurality of macromolecular structures comprise photodamage inhibitors.
101) The array composition of clause 100, wherein the plurality of analyte binding sites comprises at least 1×10⁶ analyte binding sites.
102) The array composition of clause 100 or 101, wherein the array composition comprises at least 10 photodamage inhibitors per one analyte of the plurality of analytes.
103) The array composition of any one of clauses 100-102, wherein the one or more interstitial regions comprise at least 10 photodamage inhibitors per one analyte of the plurality of analytes.
104) The array composition of any one of clauses 100-103, wherein the plurality of analyte binding sites comprises an average photodamage inhibitor density of 10 photodamage inhibitors per analyte binding site of the plurality of analyte binding sites.
105) The array composition of any one of clauses 100-104, wherein the average distance between an analyte binding site of the plurality of analyte binding sites and a nearest neighbor analyte binding site is no more than about 2 microns (μm).
106) A composition, comprising:
   a. a solid support;
   b. an anchoring moiety, wherein the anchoring moiety is coupled to the solid support;
   c. an analyte, wherein the analyte is coupled to the anchoring moiety; and a pendant moiety, wherein the pendant moiety comprises a plurality of molecular chains and a plurality of photolabile groups, wherein each molecular chain of the plurality of molecular chains is linked to at least one other molecular chain of the plurality of molecular chains by a photolabile group, wherein the pendant moiety further comprises a plurality of detectable labels, wherein detectable labels are coupled to molecular chains of the plurality of molecular chains, and wherein a quantity of detectable labels is proportional to a quantity of photolabile groups.

What is claimed is:

1. A method, comprising:
a) providing a single-analyte array comprising a plurality of sites, wherein individual sites of the plurality of sites each comprise one and only one macromolecule of a first plurality of macromolecules;
b) binding a second plurality of macromolecules to macromolecules of the first plurality of macromolecules at a fraction of sites of the plurality of sites, thereby forming a macromolecule complex at respective sites of the fraction of sites, wherein the macromolecule complex comprises a macromolecule of the second plurality of macromolecules bound to one and only one macromolecule of the first plurality of macromolecules;
c) detecting, in the presence of photons, a signal at each individual site of the first fraction of sites; and
d) after detecting, in the presence of photons, the signal at respective sites of the first fraction of sites, dissociating macromolecules of the second plurality of macromolecules from macromolecules of the first plurality of macromolecules;
wherein individual macromolecule complexes each comprise a plurality of photodamage inhibitor moieties that are coupled to the individual macromolecule complex.

2. The method of claim 1, wherein a macromolecule of the first plurality of macromolecules comprises an analyte of interest.

3. The method of claim 2, wherein the analyte of interest is coupled to an anchoring moiety.

4. The method of claim 3, wherein the anchoring moiety comprises a nanoparticle.

5. The method of claim 1, wherein a macromolecule of the second plurality of macromolecules comprises an affinity agent.

6. The method of claim 5, wherein the macromolecule of the second plurality of macromolecules comprises two or more affinity agents.

7. The method of claim 5, wherein the macromolecule of the second plurality of macromolecules further comprises a nanoparticle.

8. The method of claim 7, wherein the nanoparticle comprises a polymer nanoparticle.

9. The method of claim 8, wherein the polymer nanoparticle comprises a nucleic acid nanoparticle.

10. The method of claim 8, wherein the polymer nanoparticle comprises a branched or dendrimeric polymer nanoparticle.

11. The method of claim 1, wherein a photodamage inhibitor moiety of the plurality of photodamage inhibitor moieties is coupled to a macromolecule of the first plurality of macromolecules.

12. The method of claim 1, wherein a photodamage inhibitor moiety of the plurality of photodamage inhibitor moieties is coupled to a macromolecule of the second plurality of macromolecules.

13. The method of claim 1, wherein a photodamage inhibitor moiety of the plurality of photodamage inhibitor moieties is coupled to a surface-coupled moiety of a site of the fraction of sites.

14. The method of claim 1, wherein binding the second plurality of macromolecules to macromolecules of the first plurality of macromolecules at the fraction of sites of the plurality of sites comprises binding an avidity component of a macromolecule of the second plurality of macromolecules to a complementary avidity component of a site of the fraction of sites.

15. The method of claim 14, wherein a photodamage inhibitor moiety of the plurality of photodamage inhibitor moieties is coupled to the avidity component of the macromolecule of the second plurality of macromolecules.

16. The method of claim 15, wherein a photodamage inhibitor moiety of the plurality of photodamage inhibitor moieties is coupled to the complementary avidity component of the site of the fraction of sites.

17. The method of claim 1, wherein dissociating macromolecules of the second plurality of macromolecules from macromolecules of the first plurality of macromolecules comprises dissociating macromolecules of the second plurality of macromolecules from macromolecules of the first plurality of macromolecules at at least 95% of sites of the fraction of sites.

18. The method of claim 1, wherein the detecting, in the presence of photons, the signal at each individual site of the first fraction of sites occurs in the presence of a fluidic medium.

19. The method of claim 18, wherein the fluidic medium comprises a second plurality of photodamage inhibitors.

20. An array composition, comprising:
a) a plurality of sites, wherein individual sites of the plurality of sites each comprise one and only one macromolecule of a first plurality of macromolecules;
b) at individual sites of a fraction of sites of the plurality of sites, a macromolecule of a second plurality of macromolecules bound to the one and only one macromolecule of the first plurality of macromolecules; and
c) at individual sites of the fraction of sites of the plurality of sites, a plurality of photodamage inhibitor moieties coupled to a respective individual site;
wherein the fraction of sites contains an average of at least 100 photodamage inhibitor moieties per site.
